SEQUENCE LISTING (209 sequences)

SEQ ID NO: 1 - DNA, 401 bp, Homo sapiens
ctggcatgatgacttatgcctgtaatcccagtgttttgggaggctaaggtgggaggatcacttgaggccaggaattcaaaaccagcctggacaatacaatgagactttgtctctaaaaaaaaataaaataaattaaaataaacacagctggatgtggtggcacaggaaaaaaaaataccatttaggagtctcttaaaggcrgcttgtgaatgcttacaaagcgtggctagtatcttattacagaaaacagagcccacatcatgcatccttcttctcacatttcataaacaaggccaagggaaactgctgtggggcaacctgttgctttggtgttggtccccaagatgcagccctcacaatctgcccccaaacgtgtcagaacatgaaccccctcctccccc

SEQ ID NO: 2 - DNA, 401 bp, Homo sapiens
ttgtgttggcacatttgccacagaaagaaaacttgggataaatcctattatagaggcagtaaatattacataaagtaaaaaatcaactctcagcttatttggcttttgaaaaattctactctggggttgctcagagtgagctatatattgagattttaaaaatatttttgaagtacttatgacatacaaaataaaatattrtctacatagttaaagtcccctttgtccctccacttgctcttatgtccctccctccccactagaggcaaccctgattctgatttggcagatattatttctttaatatacctatttttggaaaagagaaacaatcgttcaaggatgcaggtagtgtgagctggaggaaaatgatatcacagttggtggggcagagtgtgtgt

SEQ ID NO: 3 - DNA, 401 bp, Homo sapiens
ggaggtcccatttgctgagcatggaattcaatgcacttgagaaaattgggctaaaaaaagaacagagcaaaagaatgtgcatgtgacaaagcaggctgacacacggaatcgctatactctggaggggctttgtttcacctgccaacaggccccttacgtgttgtgttggcacatttgccacagaaagaaaacttgggataratcctattatagaggcagtaaatattacataaagtaaaaaatcaactctcagcttatttggcttttgaaaaattctactctggggttgctcagagtgagctatatattgagattttaaaaatatttttgaagtacttatgacatacaaaataaaatattatctacatagttaaagtcccctttgtccctccacttgctct

SEQ ID NO: 4 - DNA, 401 bp, Homo sapiens
cccatgcattgcctcaggcatcttctgtgctccagatcttccttgagatcttggcttcctagggaccaatgggagttcccgggatgcttcctgctaactttcaatcccaccctcagtttccttccagaacatcctgcctttagtcctgagttctgacccctcctgtcttaacaggactcagtctttcagccccatttgagracatctactttggactctggggtttcaacagctctctggcctgcattgcaatgggaggaatgttcatggcgctcacctggcaaacccacctcctggctcttggctgtggtgagtctcccacgcccctgggggagggctgctcatgactacaggatctcaatcaaggataagcagtaaaaacggactgcatgaaaaatcag

SEQ ID NO: 5 - DNA, 401 bp, Homo sapiens
ggttgattacatttacccctttgagtgagcatcacagtaacccagccattctaaaacttcagaatgcatcagaatcacctgaaagacttgttaaaacacaaatcgctgggccccctcctcagtctgattcagcgtcagagataaggggaagaatatttctttttttatttttctaaaaaacagtctcattctgagccaagrtcgcgccactgcacttcagcctgggcaacagagcaagacttcatctcaaaaaaaaaaaaaaaagagaaaagaaaaaaaaagaaaaagggtctcattctgttgcccaggctggagtgcggtggtgtgaacacagctcactgcagcctcaacctcctgggctcaagcaatcctgcagcctcagcctcccaagtaaagtagct

SEQ ID NO: 6 - DNA, 401 bp, Homo sapiens
atttttctaaaaaacagtctcattctgagccaagatcgcgccactgcacttcagcctgggcaacagagcaagacttcatctcaaaaaaaaaaaaaaaagagaaaagaaaaaaaaagaaaaagggtctcattctgttgcccaggctggagtgcggtggtgtgaacacagctcactgcagcctcaacctcctgggctcaagcratcctgcagcctcagcctcccaagtaaagtagctaggaccacaggcgtgccaccatgcctggttaattttttattttttatagagatggggtctccctatgttacccaggctgatcttgaattcccgggctcaagcaatcctcccgcctccacctcccaaagtgctgggattacaggcataagccaccatgccggcagaa

SEQ ID NO: 7 - DNA, 401 bp, Homo sapiens
aaaaagaaaaagggtctcattctgttgcccaggctggagtgcggtggtgtgaacacagctcactgcagcctcaacctcctgggctcaagcaatcctgcagcctcagcctcccaagtaaagtagctaggaccacaggcgtgccaccatgcctggttaattttttattttttatagagatggggtctccctatgttacccagsctgatcttgaattcccgggctcaagcaatcctcccgcctccacctcccaaagtgctgggattacaggcataagccaccatgccggcagaatttccacttctaacaagttctcagggggtgctgatgctgttgctctcaggatcacatttcaagaactgctgtattaatcctttctgactcccagtgttctagccagactc

SEQ ID NO: 8 - DNA, 401 bp, Homo sapiens
gctcactgcagcctcaacctcctgggctcaagcaatcctgcagcctcagcctcccaagtaaagtagctaggaccacaggcgtgccaccatgcctggttaattttttattttttatagagatggggtctccctatgttacccaggctgatcttgaattcccgggctcaagcaatcctcccgcctccacctcccaaagtgctkggattacaggcataagccaccatgccggcagaatttccacttctaacaagttctcagggggtgctgatgctgttgctctcaggatcacatttcaagaactgctgtattaatcctttctgactcccagtgttctagccagactcagcctgtcagagcgagaaggcatcctgagacctctactccatccttcttactttact

SEQ ID NO: 9 - DNA, 401 bp, Homo sapiens
gggggtgctgatgctgttgctctcaggatcacatttcaagaactgctgtattaatcctttctgactcccagtgttctagccagactcagcctgtcagagcgagaaggcatcctgagacctctactccatccttcttactttactgttggggtcctgaggccagagaggctaagggatgtgccgcagggaatctggacagcratgggtaaatccacccccggaacccacacttaccatccacctccagagttatcccaccgcactcctctgcttcccttttatagcattcaggccctcacggcaacctcttaggtgaaaacagactgcatgtgatttggatctgaaaagctaatagatcccaggtggattttgagtggaggctcattcacccatagcctctg

SEQ ID NO: 10 - DNA, 401 bp, Homo sapiens
ctccagagttatcccaccgcactcctctgcttcccttttatagcattcaggccctcacggcaacctcttaggtgaaaacagactgcatgtgatttggatctgaaaagctaatagatcccaggtggattttgagtggaggctcattcacccatagcctctggcatgcctaattcaatcaaagtataagcatttaagataatrttctagagtggagagaatgagatttgcttgggaacaaaaaggaggagggatagtgtaatgtggagaaattatgtctaatctagtggaaatatatgtctagaatcagtttatcaccagattaatcaagccaaggtatctaaacagttatgaaaacagtgggccatgtatcaggcgggtttagaatagatttctgcactggc

SEQ ID NO: 11 - DNA, 401 bp, Homo sapiens
ttcacccatagcctctggcatgcctaattcaatcaaagtataagcatttaagataatattctagagtggagagaatgagatttgcttgggaacaaaaaggaggagggatagtgtaatgtggagaaattatgtctaatctagtggaaatatatgtctagaatcagtttatcaccagattaatcaagccaaggtatctaaacrgttatgaaaacagtgggccatgtatcaggcgggtttagaatagatttctgcactggcagaaaatgggatggtaccaacggtttctaaagacccattccattttgattcgatgctatagcaagggtaacataactcaggttgctgtgatgtagccatgtagatgtcattttgtcaaattctttactattactcagctattt

SEQ ID NO: 12 - DNA, 401 bp, Homo sapiens
ccatagcctctggcatgcctaattcaatcaaagtataagcatttaagataatattctagagtggagagaatgagatttgcttgggaacaaaaaggaggagggatagtgtaatgtggagaaattatgtctaatctagtggaaatatatgtctagaatcagtttatcaccagattaatcaagccaaggtatctaaacagttaygaaaacagtgggccatgtatcaggcgggtttagaatagatttctgcactggcagaaaatgggatggtaccaacggtttctaaagacccattccattttgattcgatgctatagcaagggtaacataactcaggttgctgtgatgtagccatgtagatgtcattttgtcaaattctttactattactcagctatttcacct

SEQ ID NO: 13 - DNA, 401 bp, Homo sapiens
aatcaaagtataagcatttaagataatattctagagtggagagaatgagatttgcttgggaacaaaaaggaggagggatagtgtaatgtggagaaattatgtctaatctagtggaaatatatgtctagaatcagtttatcaccagattaatcaagccaaggtatctaaacagttatgaaaacagtgggccatgtatcaggygggtttagaatagatttctgcactggcagaaaatgggatggtaccaacggtttctaaagacccattccattttgattcgatgctatagcaagggtaacataactcaggttgctgtgatgtagccatgtagatgtcattttgtcaaattctttactattactcagctatttcacctagctgttctgttgaaatgttgaact

SEQ ID NO: 14 - DNA, 401 bp, Homo sapiens
tgtctagaatcagtttatcaccagattaatcaagccaaggtatctaaacagttatgaaaacagtgggccatgtatcaggcgggtttagaatagatttctgcactggcagaaaatgggatggtaccaacggtttctaaagacccattccattttgattcgatgctatagcaagggtaacataactcaggttgctgtgatgtrgccatgtagatgtcattttgtcaaattctttactattactcagctatttcacctagctgttctgttgaaatgttgaactccttctccatattcgttcacaaggataaaggagaggattacagacaggtgctgtagccacctgagttcagctgggttggaatgtttatcctacaacctttcagctttattctgagattggt

SEQ ID NO: 15 - DNA, 401 bp, Homo sapiens
gtagccatgtagatgtcattttgtcaaattctttactattactcagctatttcacctagctgttctgttgaaatgttgaactccttctccatattcgttcacaaggataaaggagaggattacagacaggtgctgtagccacctgagttcagctgggttggaatgtttatcctacaacctttcagctttattctgagattsgttaggggtttccacctgagttcagctgggttagaatgtttatcctacaacctttcagctttattctgagattggttaggggtttcaaacctttatttgggatgcatacctttatttttctggaggaagtagccacaaatatgtattaaacacacatgatacaaaagacagtaccaggaagagcaaggggtttagaagct

SEQ ID NO: 16 - DNA, 401 bp, Homo sapiens
gggacagcctgagcatgtccttcaagatcaaggagaaggcattttgagcacaggagatggcgacgaggtttttgtttttctgggttttttgttgttttttgttttttggtttttttttttttttttttgacagagtcttgctctgttgccaggctggaatgcagtggcacagtggcacgatcttggctcactgcaacctcygactccctggttcaagcggttctcctgcctcagcctcccaagtagctgggcttacaggcacgcaccatcacgcctagctaatttttgtatttttagtagagacggggtttcaccatgttggccaggatggtctcaatcttctgacctcatgatctgtccaccccggcctcccaaagtgctgggattacaagtatgagcca

SEQ ID NO: 17 - DNA, 401 bp, Homo sapiens
ttctgggttttttgttgttttttgttttttggtttttttttttttttttttgacagagtcttgctctgttgccaggctggaatgcagtggcacagtggcacgatcttggctcactgcaacctccgactccctggttcaagcggttctcctgcctcagcctcccaagtagctgggcttacaggcacgcaccatcacgcctasctaatttttgtatttttagtagagacggggtttcaccatgttggccaggatggtctcaatcttctgacctcatgatctgtccaccccggcctcccaaagtgctgggattacaagtatgagccaccgcacctggcgggtgctgagttttttgttttatgttgttgttgttgtttgagatggactcttgctctgtagctcag

SEQ ID NO: 18 - DNA, 401 bp, Homo sapiens
tgggccttctcagaggaagtaaaggcaggcttgggtatcagtcatatttggggagaagatacagatgttgataaacttaagcaattgagaggagaaaataaacaagcttgattcttaataagttatgcaaatccaccagaaaaactgtttttccagacttttaaatagtgctgagcgagtgctagacactatttgttctaygagtttcagacatgcattaactaacttaatcatcagaacaactgtatgaattaaatatggtaacttccattttctaagatggggaaactgaggcatagaaaggttaagtaatttgcttacattcataagcaaatacgctagtaagtagcagacctgctggaattgaatccaggctgtctggcaccagagtctgagctctt

SEQ ID NO: 19 - DNA, 401 bp, Homo sapiens
ttttattttggatttgtcgtaagtataagtttttgttttgggtacttgcttatttaggcaactgtaaactttattaacttgcttattcactctgacttagttcatattaaccttctgtacttttttttttttgagacagagtctcactctgttccccaggctggagtgcagtggcacaatctcagctcactgcagcctccrcctcctgggttcaagcgattcctatgcctcagactcccaagtagctgggattacagacatgcaccaccatgcccagctaattttttgtactttttgtagagacagggttttgccatgttggccaggctggtctcaaactcctgacctcaagtgatccacctgcctcggcctcccaaagtgctaggattactggtggatta

SEQ ID NO: 20 - DNA, 401 bp, Homo sapiens
cagcactttgggaggccaaggcgggtggatcacaaggtcaagagattgagaccatcctggctaacacggtgaaaccccatctctactaaaaatacaaaaattagctgggcgtggtagcacgcgcctatagtcccagctactcaggaggctgaggcaggagaaccacttgaactcggaaggcagagctgcagtgagctgagrtcatgccactgcactccagcctgggtgacagagagagactctgtctcaaagaaaaaattatcgactgtaggttgttcagtttgttgtccttcttttatggtatttgctctcctgggatgtcccctttccttgtcctgggagctcacgtttccctcgggataccagctgtttgggtgagtctctgggcagagatggaagcc

SEQ ID NO: 21 - DNA, 401 bp, Homo sapiens
acatgaagacgagatggtgagtgtgtctcacggtgagctccggtggcccaagtggctgtgtggccattatatgaaggtcattcttcaggctgtccccatgaaacctgagggcttccctgagcctctgtgagccttctcttcaaccaaaactgaggaatagataattagctggttgagatctttgcttttgttgttttacaytgaaagtcacccatatactcgaattactgattctacaattttttggccactcaaagcaaataaaaacataagacgttggctgggcgcggtggctcatgcctgtaatcccagcactttgggaggccgagacgggcagatgacaaggtcaggagattgagaccatcctggttaacatggtgaaaccccgtctctactaacaa

SEQ ID NO: 22 - DNA, 401 bp, Homo sapiens
caacaccatgaaaaatatattcctcttacttccattgtacaggtgaggaaatggaggcttaaaacagagcccatggagctcctaagtgatggagccaggatttgaacccaggactgctgactttaggctcatgcttgtaatcagggcactgtgcattccaggtgatttatattggaaggcagcctttcctgtgattaaaartgcatctacgaagcattgttctttccctcctttttttttctgtagccctgttcacggcctatcttggagtcggcatggcaaactttatggctgaggtgagtttgctttagtctcacttttcattagcgtaattgaccagcttacaactatatgggaaatgctcctgaagtccactgggctggcatccagtggcaggatcc

SEQ ID NO: 23 - DNA, 401 bp, Homo sapiens
tgctgctgccaaaagggctagatactgccttgactccttagcagccatgaccacatcaacatggactaagctatcagaaaccggccctgcatcacctgcaatggaatatctggctttctgtttaagtaaaagatatcctaaagtctacttcttatcacttagagttagactctgaacttgataatatgttatacaacctaygtttcatttttttttaaaccaagcaatctgatacttgtaacaactgggtgattgtggtttggactaaagtttgctgtgatgctgaaagaaaggccagggagctccaggagaagggagagcagtgcttctcatggtcatggatcctgccactggatgccagcccagtggacttcaggagcatttcccatatagttgtaagc

SEQ ID NO: 24 - DNA, 401 bp, Homo sapiens
tgaagggccccatgtgctctgatgtactctccatcatgcatgtgtgtaatgggactttggctttccaccagaaagacactgtgtctccttaaggaccctcccagataaaatctccatgctgctgccaaaagggctagatactgccttgactccttagcagccatgaccacatcaacatggactaagctatcagaaaccggscctgcatcacctgcaatggaatatctggctttctgtttaagtaaaagatatcctaaagtctacttcttatcacttagagttagactctgaacttgataatatgttatacaacctacgtttcatttttttttaaaccaagcaatctgatacttgtaacaactgggtgattgtggtttggactaaagtttgctgtgatgctg

SEQ ID NO: 25 - DNA, 401 bp, Homo sapiens
ggcaagctgcacaccagagtcagctaggaagacagaaaaatatggagccttaggccctgtcctttggtatttctgatagagtaggtcttgtatgatgcttgaacatctgtgtttttttttaactcccccagatgattctgatgtgcagtcagattagggtacccctacactccatcacaccccagggaggtccatgcatcrggtcagagctaaccaatggtgtatgctcagaattgtgtgagtttccatgagcagcacaaagaggacctaccctcaaggaacttagagtctatttgggagacagaatggaaagaaacaaagcaagtcaagtctaagatctagaccaggcagaagtcaaggtcagagaggtcactgtgggctggactaatcagagaaggcct

SEQ ID NO: 26 - DNA, 401 bp, Homo sapiens
ttctctccctctcatatattcagacttcctgcaccagatgtgccaagagaacatttgacagagatgacacttgcaaactgcaaatggcccctgaccagtcttcatgtccacaaggccttctctgattagtccagcccacagtgacctctctgaccttgacttctgcctggtctagatcttagacttgacttgctttgtttytttccattctgtctcccaaatagactctaagttccttgagggtaggtcctctttgtgctgctcatggaaactcacacaattctgagcatacaccattggttagctctgacctgatgcatggacctccctggggtgtgatggagtgtaggggtaccctaatctgactgcacatcagaatcatctgggggagttaaaaaaaa

SEQ ID NO: 27 - DNA, 401 bp, Homo sapiens
caatagcgtggccaaacagaagggccaggtacaagctggcaatccaacctggggaagaagagaaagccacacgctcctttcagatcccatctgacagctgagctgcacaaggcatccccctctacttgcattcagcccagggggatgaaccactgggggcattgctctggattctgtgcatgctcgtggagactgaatttmcaatctcagggcattgagttgttggaatgttgacatccattctttagggcagccacttatgtctctatcttgtatgtctttctctccctctcatatattcagacttcctgcaccagatgtgccaagagaacatttgacagagatgacacttgcaaactgcaaatggcccctgaccagtcttcatgtccacaaggccttct

SEQ ID NO: 28 - DNA, 401 bp, Homo sapiens
aacctgttcttacatattaaagaaaagttacttactgtatttatgaaatactcagcttaggcatttttactttaacccctaaattgattttgtaaatgccacaaatgcatagaattgttaccaacctccaaagggctctttaaaatcatattttttattcatttgaggatgtcttataaagactgaaggcaaaggtcagawtgcttacgggtgttatttttataagttgttgaattccttaatttaaaaaagctcattattttttgcacactcacaatattctctctcagaaatcaatggcatttgaaccaccaaaaagaaataaagggctgagtgcggtggctcacgcctgtaatcccagcactttggggagcccaggcgggcagattgcttgaacccag

SEQ ID NO: 29 - DNA, 401 bp, Homo sapiens
ttgcttgaacccaggagttcaagaccagcctgggcagcatggtgaaaccctgtatctacaaaaaatacaaaaattagccaggcatggtggtgggtgcctgtagttccagctacttgggaggctgaggtgggaaaatgacttgagcccaggaggaggaggctgcagtgagctaagattgcaccactgcactccaacctgggygacaagagtgaaactgtgtctctcaaaaaaaaaaaaaaacaaacaaaaacaaaaacaaaacaaaacaaaacaaaacaaaacaggtaaggattcccctgttttcctctctttaattttaaagttatcagttccgtaaagtctctgtaaccaaacatactgaagacagcaacagaagtcacgttcagggactggctcacacc

SEQ ID NO: 30 - DNA, 401 bp, Homo sapiens
tgggcaacatagcaagactccatctcttaaaaaataaaaatagtaacattagccaggtgtagcagcacacatctgcagcagctactcaggaggctgaggtggaaagatcgcttgtgcacagaagttcgaggctgcagtgagctatatgatcatgtcactgcactccagcctgtgtgaccgagcaagaccctatctcaaaawaattaattaattaattaattaattaatttaaaaaggaagtcatgttcatttactttccacttcagtgtgtatcgtgtagtattttggaggttggaaagtgaaacgtaggaatcctgaagattttttccacttctagtttgcagtgctcagtgcacaatatacattttgctgaatgaataaacagaaatagggaagtaaac

SEQ ID NO: 31 - DNA, 401 bp, Homo sapiens
tgcatagaaatttgttatacagacattaggaaaaagtataagtgctccctgcaaatattcaattttaggaaaatagtaacaaaaatagcttgagaaaattaatatcatttaaattcgacaatgtacaatgttcccatttacagatttgtgtgagcacaagaagctcctgcatggtagatccctgttctgaagatcgactcrggtccattttagaggcctctggttctctcagccacagcctagattcttcactgctgatgatctttggttatgcttgcttttttttccccacaattcttctttcacacgttccctaacaagaaagggttcagttcaaacactgaaaattgagcttctccacagcaatatccataaacctatgacagttgcaaattcatagt

SEQ ID NO: 32 - DNA, 401 bp, Homo sapiens
taccatgcaggagcttcttgtgctcacacaaatctgtaaatgggaacattgtacattgtcgaatttaaatgatattaattttctcaagctatttttgttactattttcctaaaattgaatatttgcagggagcacttatactttttcctaatgtctgtataacaaatttctatgcaagtacatgaataaattatgctcacmgctcagttttgtataatgtgccatttatagccatcatgtatatgaacaatcacaggcataagtaggtatcgcttatgttgcacgtgctagaatccttattcctttcttacacttggtaatagcaccagatgtatagttgagtaaacccttttcaatttcagattggcaggcccatccccttgggtatcctctcaactatc

SEQ ID NO: 33 - DNA, 401 bp, Homo sapiens
aagatgagaaatttacttcaacaagtttgaatgataagtttcaaagcagccctaaaaggaaactaaagaagccatagtggggtctccctattgaagtgccctctgtcataacaaaacttctcctacctatggtttcaaggtaaattcaaataaattctccagagggatctgggcattctatgtcttcttttatctggggtrgaaacgacaggttgtgaggctcttatgctaaggacactctatacctttaggagctaaaggggccaacttgcctgttacccatgatacaggattagcacggaatatgtcaagatcaaagctaagttttacaagaaaccaggaatttctaaggaggcttcttattaacaattttgcaagaatatttttgtggttagtaaaca

SEQ ID NO: 34 - DNA, 401 bp, Homo sapiens
ctaaaagagaaaattgctcccaggactgacctaatcttatttaatattaacctggtaggtgtgaacatatctaaagcaagcttaggtccacctgatggggtagcaagtcaaccactgaggctcaggaagaggaaaacctaggaagggggtcttcaatattggaacttggggtgggacaggtgtcaagggtgagctctgtgrcggaaatggcactggggtattatggtttagtaaactgaactatatagaccaagcaactaatgctccttgtccaatcctgtactcacaagctatttggcttcctatgtagacttgtcttacagggtaaaaacacatgtgtccacactgaggaagggaggtttaggcagagatctaaggaataggcaggaggtggaggaagg

SEQ ID NO: 35 - DNA, 401 bp, Homo sapiens
gcgaggaagagtgtgcagcatgagaggagaagtgatccttcagtgctccatcgatggtgtggcaccaccattcaccttgtagcccaagctacaaatcaggaagccatccttgacacctccccctccctcaccctgcaaatcaactcatcacagagttctggtgattctgcctcctgtctctcatatccatgaacgtctctmcatcaccactgaagtccaagccaccatcacttctctcctgcactactgcaatggcctcccagttggcttgctgaattcactccagcaccctttcattcaccataatccagccacagcaatcttctcactccttgaaagcaccaatctccttgtttcaaggcctttatccacaagtttccctttgccaagggtactgttcc

SEQ ID NO: 36 - DNA, 401 bp, Homo sapiens
cttcaactccctacagggactgttccaaaagcacttcctcaaactcaacatatccaggacaaaactcatcttcttcaactattcttctctccctcccatgttgactcatggagaaaggtagcaccttccacccgaatgggaaagccagaaacaggatgccatccttgacctcattctctcacctccaggaaatagttagcyaagggctgagggttccacctccttgatagcccccattcatccatttattgaatgactgtttactacataacaggaactgtgactacaatagtgaaccagacggccatagtctcttccctcacaaaaactttatgaggtgaactgacaagtaaaggagtaatataatatagtgcagtgatgataatgagtaagagacaacc

SEQ ID NO: 37 - DNA, 401 bp, Homo sapiens
acagggactgttccaaaagcacttcctcaaactcaacatatccaggacaaaactcatcttcttcaactattcttctctccctcccatgttgactcatggagaaaggtagcaccttccacccgaatgggaaagccagaaacaggatgccatccttgacctcattctctcacctccaggaaatagttagccaagggctgaggkttccacctccttgatagcccccattcatccatttattgaatgactgtttactacataacaggaactgtgactacaatagtgaaccagacggccatagtctcttccctcacaaaaactttatgaggtgaactgacaagtaaaggagtaatataatatagtgcagtgatgataatgagtaagagacaaccagttaacaagtt

SEQ ID NO: 38 - DNA, 401 bp, Homo sapiens
aactcaacatatccaggacaaaactcatcttcttcaactattcttctctccctcccatgttgactcatggagaaaggtagcaccttccacccgaatgggaaagccagaaacaggatgccatccttgacctcattctctcacctccaggaaatagttagccaagggctgagggttccacctccttgatagcccccattcatycatttattgaatgactgtttactacataacaggaactgtgactacaatagtgaaccagacggccatagtctcttccctcacaaaaactttatgaggtgaactgacaagtaaaggagtaatataatatagtgcagtgatgataatgagtaagagacaaccagttaacaagtttgagggaagagtatgccagtgacctcttt

SEQ ID NO: 39 - DNA, 401 bp, Homo sapiens
agttaggatgcctttgcctgcaagtaacaaaatacctgacagaaagtggttaaaacaacaggaatttcattcctaaaagtaggaagttccagattttgtgctcaatgatgtcaccaaggggtgcaatttctttccatcttcctcatcattctcagcatcctcagaaggctccactttcatcctagagcttgtagcctcatwgttacaacatggctgccctggttacaaacatcacaaccacatcaagaagaaggggggcaaatagagttgcaagagggccattctctcatgtttctcttctttatcagggagggaagtctttcttagaagctcctctaaaataatttcacttgtgtctttttggcagaactgggttgtatggccattcatggctgcctaaa

SEQ ID NO: 40 - DNA, 401 bp, Homo sapiens
ctcttgtaacaggtgaactctgccatattggagcaaaagcaagaggaatggcaattggacaggcagccaacatggctgctacagtggtatatagtaccaccatatagcatgtagttcctatgtgcccacacaagagagagacatagtgataggttagactgtctattgtgagattcacaaaggggtttggaacagaaatcyagaccaagaccttttacacaagagattctttgtggaatacttgctggaatattcccagagtattttgggataccccagactcatacaaggttgtataacctctaactaacaagattctaagcaaaggatgagacgtgatcatgctcaggtaacgaaaacttagggtcaattcatgttctaattagttctcttgatctaac

SEQ ID NO: 41 - DNA, 401 bp, Homo sapiens
aagaccttttacacaagagattctttgtggaatacttgctggaatattcccagagtattttgggataccccagactcatacaaggttgtataacctctaactaacaagattctaagcaaaggatgagacgtgatcatgctcaggtaacgaaaacttagggtcaattcatgttctaattagttctcttgatctaacagttayggcttataaatcattccatgtcggaagccccaccggaaatagttgaggttcaattcagttacagctcaatgaagtctgaaaacaagtgtccaacattttctggttcatggtttaaaataggtttcaaataaacaatgaggaagccagtttcctgtttgggttgggtccattggatcctagcccatcaaagctttgaatta

SEQ ID NO: 42 - DNA, 401 bp, Homo sapiens
tgaggttcaattcagttacagctcaatgaagtctgaaaacaagtgtccaacattttctggttcatggtttaaaataggtttcaaataaacaatgaggaagccagtttcctgtttgggttgggtccattggatcctagcccatcaaagctttgaattatattacaatgacaggcaaggactagagggggaagaactgaaacrcagagaaaagttggcacagtgccaggaaacctggctaaaattaagtccctcagtccaaagaaaacaatggcagctaggacatgagtcaatggtccaagtgcaagtgcatttgggggcaacaagagcacacacaggcaggccatggaactggccaacccaacaagaaggtctttccacattctgagatcccccctaatgct

SEQ ID NO: 43 - DNA, 401 bp, Homo sapiens
ctccagaagtttgagttccaacagctcagtgccatctaagttgctatggagactttgtaaaccactaagaatcctgggctggggaactgtctagcccagtgctgttgaatagggatacaatgtgagccacaaatgcaagttatttgtataattttggattttcaaaagtggccatcttaaaaatgtatttaaaaaggtgarattaattttaacaatatattttattgtcatttcaaaatttaatatagacaattattgagatattttttccttttttgttataagtgttgggaacccagtgtgcaatttacatgtagaacacatctcaatttggaccagctgcactgcaagtgctgcaccatggatagtggctactgtattcaatagtacagatctaccct

SEQ ID NO: 44 - DNA, 401 bp, Homo sapiens
tacaatgtttcacaaccaggggaatttatcagaattatgcctaagggcctttgaaaaacttcagttattcccatctcttctcagacctcctgaatctgaatttctaaagtttcgggggacctgccccgaaaatcacgtaggttcttttctattttcctaagtgtcggctggcttgagaaataaagggacagagtacaaaakagagaaattttaatttctgggcgtccgggggagacatcacacgttggtaggatccatgatgccccacaagccacaaaaccaagcaagtttttattagggattttcaaaaggggagggagtgtgcgaataggtgtgggtgacagacatcaagtacctaacagggtaatagaatatcacaaggcaagtggaggcagggcgag

SEQ ID NO: 45 - DNA, 401 bp, Homo sapiens
attaaaattgctaatggagtttcgggcaccattgtcattgataacatctcatcaggagacagggttttgagatcaaccggtctgaccaaaatttattaggcgggaatttcgtcttcctaataagcctgggagcgctatgggagactggagtctatctcacctctgcaatctcgaccataagagacaggtacgccccaggarggccagttcagagacctacccctaggtgcgcattctctttctcagggacgttccatgctgagaaaaagaattcagcgatatttctcccatttgcttttgaaagaagagaaatatggctctgttctgcctggctcactggcagtcagagtttaagtttatctctcatattccctgaacaattgctgttatcctgttctttt

SEQ ID NO: 46 - DNA, 401 bp, Homo sapiens
agaaattataaaagtattaatttggggaactaataaatgtccataaaatcttcacaatccacgttcttctgtcatggcttcagctggtccctctgtttggggtctctgacttcccgcaacactaaaggtactacagatgattcgaaaagtgaaaggaaaataaatctcgagaccccaaaatcattaagccaaagggaaaarttaagctgggaactgggtcacgcaaaactgcctcccctttggttcctaaattggatggctacaagatgaaaagctacacactgccctcatattttgcccacagggaaatccctagtgaactccaagatctttaaagtgtttctgttaaaattcaccatggtgcccaggcacagtggctcacgcctgtaatcccaggactt

SEQ ID NO: 47 - DNA, 401 bp, Homo sapiens
ttaagctgggaactgggtcacgcaaaactgcctcccctttggttcctaaattggatggctacaagatgaaaagctacacactgccctcatattttgcccacagggaaatccctagtgaactccaagatctttaaagtgtttctgttaaaattcaccatggtgcccaggcacagtggctcacgcctgtaatcccaggacttygggaggccaaggtgggcggatcatgaggtcaggagattgagaccatcctggctaacacggtgaaaccccttctctactaaaactacaaaaaattagccgggtgtggtggcaggtgcctgtagtcccagctactcgggaggctgagacaggagaatcacttgaacctgggagggggaggttgcagtgagccaagatcgcac

SEQ ID NO: 48 - DNA, 401 bp, Homo sapiens
tgggaggccaaggtgggcggatcatgaggtcaggagattgagaccatcctggctaacacggtgaaaccccttctctactaaaactacaaaaaattagccgggtgtggtggcaggtgcctgtagtcccagctactcgggaggctgagacaggagaatcacttgaacctgggagggggaggttgcagtgagccaagatcgcaycactgcactccagcctgggtgacagagcgagactctgtctcaacagaaaaaaaaaaaaattaactatggcaatgtaaatgataacttatctttacaggtgcaatcacccctcttcccacctgatacaaatgcatatctgattattcccacccacccaccccccaccgccccatttgtctgttaatcttatgtaaaagtgc

SEQ ID NO: 49 - DNA, 401 bp, Homo sapiens
catcctggctaacacggtgaaaccccttctctactaaaactacaaaaaattagccgggtgtggtggcaggtgcctgtagtcccagctactcgggaggctgagacaggagaatcacttgaacctgggagggggaggttgcagtgagccaagatcgcaccactgcactccagcctgggtgacagagcgagactctgtctcaamagaaaaaaaaaaaaattaactatggcaatgtaaatgataacttatctttacaggtgcaatcacccctcttcccacctgatacaaatgcatatctgattattcccacccacccaccccccaccgccccatttgtctgttaatcttatgtaaaagtgcagattccttgcatttcccctgtcccatgtggccatgttacataa

SEQ ID NO: 50 - DNA, 401 bp, Homo sapiens
atcgcaccactgcactccagcctgggtgacagagcgagactctgtctcaacagaaaaaaaaaaaaattaactatggcaatgtaaatgataacttatctttacaggtgcaatcacccctcttcccacctgatacaaatgcatatctgattattcccacccacccaccccccaccgccccatttgtctgttaatcttatgtaraagtgcagattccttgcatttcccctgtcccatgtggccatgttacataaaaatgcagattcactgagctagacaaagacatgaatattttctccctacccacctcttgcatgaaaattgtgtacttctcactatcctgccctttcctctttaaatctggagccctcaaaatcatcttcagagaaaagcatagacctgtc

SEQ ID NO: 51 - DNA, 401 bp, Homo sapiens
cccccttcctgcacacctcgaattgaccaccactgtctgctcatcctaggaacctccaaattcaggccctttcaaccctcaggactccagattattcttccctatacccacatctaggactgacccactgctgattcacaccagtgcagacattcaaattattaaaatattcagttcttttgcacactttgtggtaagtarccactgccctgagccctgtcacttgagtctctctatgaccttcaggaatttctccacaatccagcaactaaagggttaacacaaagcacagctgtgagttccagcaatcgttctgattggtcagtccccttgccttaccaggtgattaaggttttaaatttcactgtggcaccacacggcagataaattttgtatgccta

SEQ ID NO: 52 - DNA, 401 bp, Homo sapiens
ctcacacctcatttgactaacgagtcaaatccttgctgactggcttgtctcttcactgcaatatgttcagaggaacacgactttaaatgatgcattgtcctgggtttcctcttgtacacccctcttatgcctcattcctttcctttgcatgatactcccctagttctggatggccaccttccttggatttcccatgaaaastaatctttctagagtctttttcctataggactttctctatttttttattttattttatttatttattttttttgagacggagtctctctctgtcacccaggctaaagtgcaggggtgtgatcttggatcactgcagcctctgcctcccaggttcaagcaattgtcctgcctgagcctcctgagtaactgggattacaggt

SEQ ID NO: 53 - DNA, 401 bp, Homo sapiens
gagacaagccagtcagcaaggatttgactcgttagtcaaatgaggtgtgagcccaggctaagaagcagcttccctcctccatgccatttacgtggttacttcctcttcttgtaatcttcaaatcccagcttgcctgctctaatggccatttgttgtttttgtcttccccgcatgcttcccaccttcttctagtaacagcgmctccccttccttaagggatctcctccttccccatccatggggtcctctgaggctgctggcctcaataccataatctcaaacacagtggagtcagtgactcagctgtaatcaatcatagtatctcaatcccctggccatggggattggtccaagggacagccacctaacccaaactgaggcaataagattcttccctgcag

SEQ ID NO: 54 - DNA, 401 bp, Homo sapiens
tgtgtacttagatttcaaattcctgcagggaagaatcttattgcctcagtttgggttaggtggctgtcccttggaccaatccccatggccaggggattgagatactatgattgattacagctgagtcactgactccactgtgtttgagattatggtattgaggccagcagcctcagaggaccccatggatggggaaggagsagatcccttaaggaaggggagtcgctgttactagaagaaggtgggaagcatgcggggaagacaaaaacaacaaatggccattagagcaggcaagctgggatttgaagattacaagaagaggaagtaaccacgtaaatggcatggaggagggaagctgcttcttagcctgggctcacacctcatttgactaacgagtcaaa

SEQ ID NO: 55 - DNA, 401 bp, Homo sapiens
cacctaacccaaactgaggcaataagattcttccctgcaggaatttgaaatctaagtacacagacatccaggatgaagggaaatcgaggctgggtctgagaatagtgctctaagagaaaggccatgagatacccagagccatcctggtttctttccctcccaagcccaggttactattaccttgatgtaataacaacattractatcccccagccccatatctttccaataaactgaattggtacttaagttagaatctgtttgtgtcatttcaaacacaaccaaaattgatacagaatattacaagtgacatctctagaagatggaataggcagagtcaagaaggggcggggatctgacagttcttgctacatggttaaatgattagctgatacatattg

SEQ ID NO: 56 - DNA, 401 bp, Homo sapiens
gcaagcaccactatgccctgctaatttttcatatttttagtagagacagggtttcaccttgttggccaggctggtctcaaactcctgacctcaggtgatccaccagcctcggcctcccaaagtgctgggattacaggcatgaaccaccatgcctggctttttttttttttttttttttttaagacagagtctcactctgtygccaaggctggaatgcatggatgcgatctcggctcactgtagcctccgccttcccagttcaagtgattctcctgcctcagcctccctagtagctgggattacaggtgtccaccacatttttgtatttttagtagatacgggttttcaccatgttggccaggctggtcttgaactcctgacctcaggtcatctgcctgctt

SEQ ID NO: 57 - DNA, 401 bp, Homo sapiens
gggtttcaccttgttggccaggctggtctcaaactcctgacctcaggtgatccaccagcctcggcctcccaaagtgctgggattacaggcatgaaccaccatgcctggctttttttttttttttttttttttaagacagagtctcactctgttgccaaggctggaatgcatggatgcgatctcggctcactgtagcctccrccttcccagttcaagtgattctcctgcctcagcctccctagtagctgggattacaggtgtccaccacatttttgtatttttagtagatacgggttttcaccatgttggccaggctggtcttgaactcctgacctcaggtcatctgcctgcttcggcctcccaaagtgctgaaattacaggcatgagccaccatgcccagc

SEQ ID NO: 58 - DNA, 401 bp, Homo sapiens
ttcaccttgttggccaggctggtctcaaactcctgacctcaggtgatccaccagcctcggcctcccaaagtgctgggattacaggcatgaaccaccatgcctggctttttttttttttttttttttttaagacagagtctcactctgttgccaaggctggaatgcatggatgcgatctcggctcactgtagcctccgcctycccagttcaagtgattctcctgcctcagcctccctagtagctgggattacaggtgtccaccacatttttgtatttttagtagatacgggttttcaccatgttggccaggctggtcttgaactcctgacctcaggtcatctgcctgcttcggcctcccaaagtgctgaaattacaggcatgagccaccatgcccagcccaa

SEQ ID NO: 59 - DNA, 401 bp, Homo sapiens
ccaaagtgctgggattacaggcatgaaccaccatgcctggctttttttttttttttttttttttaagacagagtctcactctgttgccaaggctggaatgcatggatgcgatctcggctcactgtagcctccgccttcccagttcaagtgattctcctgcctcagcctccctagtagctgggattacaggtgtccaccacrtttttgtatttttagtagatacgggttttcaccatgttggccaggctggtcttgaactcctgacctcaggtcatctgcctgcttcggcctcccaaagtgctgaaattacaggcatgagccaccatgcccagcccaacccactgttttcctttgcctgttggattctgccaataggatgaaatacagagagaatggaggga

SEQ ID NO: 60 - DNA, 401 bp, Homo sapiens
gatgcgatctcggctcactgtagcctccgccttcccagttcaagtgattctcctgcctcagcctccctagtagctgggattacaggtgtccaccacatttttgtatttttagtagatacgggttttcaccatgttggccaggctggtcttgaactcctgacctcaggtcatctgcctgcttcggcctcccaaagtgctgarattacaggcatgagccaccatgcccagcccaacccactgttttcctttgcctgttggattctgccaataggatgaaatacagagagaatggagggaagagggatttgcccctttcagcagtagtttccagctccttgtcactttgccacaacctgacccatcaggcccccttaaagggtgcagcaccgctaagtcattca

SEQ ID NO: 61 - DNA, 401 bp, Homo sapiens
aacccactgttttcctttgcctgttggattctgccaataggatgaaatacagagagaatggagggaagagggatttgcccctttcagcagtagtttccagctccttgtcactttgccacaacctgacccatcaggcccccttaaagggtgcagcaccgctaagtcattcattccttcctcagtgagttctgtccctggccstaattaaaattaatgtcctgactaggaagtgactacccgggaggattaaaagaaatgggcaagtctgatcccaagcaccttctattatcaatgttttctttgccaaaaaaaaaaaaaaaaatgctagcaattcctggccacaattaagtgtcactcacttgggaagagcctggggacagaggccgtactgaccttatacc

SEQ ID NO: 62 - DNA, 401 bp, Homo sapiens
agaaagcaagctctaggccatctggggagggcattctgttgtcctaggcacttggagcatggtccagaagagagtcccaggctccaggtggacatatcctcttgggcccatgcgcttcttgtccatagggaaggggcagaggaatggctagagggaggtcagtttgggacattttaaagcattgaggggtcagtatagggyccttctagcataggtataaggtcagtacggcctctgtccccaggctcttcccaagtgagtgacacttaattgtggccaggaattgctagcatttttttttttttttttggcaaagaaaacattgataatagaaggtgcttgggatcagacttgcccatttcttttaatcctcccgggtagtcacttcctagtcaggacat

SEQ ID NO: 63 - DNA, 401 bp, Homo sapiens
acaacagaatgccctccccagatggcctagagcttgctttctgggcccatgaggcccccttccctgagttctccttctaaaaggggattgggtttccaggttacacaccccttagcctgaggtgctactaagagatgctgatggaggctgggggtgaatggagcttggacatgcccactgagttgtccatatgcttgcatrgggccccactaagcagaacagagtccagtggaaagagaagatggggtgggcaagggacttctttttgtgttcttgccccagcctcacaaagaggctcagagagggcctcacaggaagtgagtaagttccaggccctggatctaaagcaaaggccaacaagatcattaagccgagatctaaggaaatgagctaagagacag

SEQ ID NO: 64 - DNA, 401 bp, Homo sapiens
ggctcagagagggcctcacaggaagtgagtaagttccaggccctggatctaaagcaaaggccaacaagatcattaagccgagatctaaggaaatgagctaagagacagaactgcaaggctgggcacagattcaaccaatgaaatgagtgagagttttatgaggctgtattgccagagaaaccttaagctgaacatataggygcttatgattgtaaaccccaagcaccagaacccatgccagaagggagcgggcctcaaggcagggccaccccagcagcaaaatcttcccaggtgctcctcaggagtgtccaaggtgagccctggatgggagaccccagcaacagggaagccatactcactgaccacagactcacccacaaccaggccgtgagtcaagaaac

SEQ ID NO: 65 - DNA, 401 bp, Homo sapiens
attcaacctattacacatatataatatgtcctaccattgcccacttgagagggcttggttgttttacatgccaaaagcagtgagcatacctaccactaagagcgtggcttctaaatgccattcttaaaacctgcacattctccacatgaatcccagaacttaaagtaacaacaacaacaaaaaagaaccaaatagtgatckaagaatgatcggggaaaactgaaaaagacacagaagtcagtttgaaagaacttccactggctaaatttgggacagtttgagcatgaaaataaataataacagtaatatgtcctctcaaataatatagaaaaccatgaatcaatacttatatacctaaatacatacatacaaacaaacatacatacatgtgctaattgaaa

SEQ ID NO: 66 - DNA, 401 bp, Homo sapiens
tcatttaagtgttctattactgattcaagttaattccaatgctttactattttaatagtaatgcaatccattttttattttataatattttagactcagggggtacatgtgcatgtttgttacatgagtatattttgtatttgtggggattgggcttctactgtaccctttacccaaatagtgaacattgtactcaatggktaatttttcaaccctcgccctcctcccaacctccccccttcaggagtccccactgtctattatttctatctttgtgttcatgtttacccattgtttatctcccacttatgagtgaaaacacgtggtatttgatttcctgagttagttcactaaggatactgccctccaattccatccatattgctgcaaaggacatgatt

SEQ ID NO: 67 - DNA, 401 bp, Homo sapiens
ctattactgattcaagttaattccaatgctttactattttaatagtaatgcaatccattttttattttataatattttagactcagggggtacatgtgcatgtttgttacatgagtatattttgtatttgtggggattgggcttctactgtaccctttacccaaatagtgaacattgtactcaatgggtaatttttcaacmctcgccctcctcccaacctccccccttcaggagtccccactgtctattatttctatctttgtgttcatgtttacccattgtttatctcccacttatgagtgaaaacacgtggtatttgatttcctgagttagttcactaaggatactgccctccaattccatccatattgctgcaaaggacatgatttcatttgttatgg

SEQ ID NO: 68 - DNA, 401 bp, Homo sapiens
aatgctttactattttaatagtaatgcaatccattttttattttataatattttagactcagggggtacatgtgcatgtttgttacatgagtatattttgtatttgtggggattgggcttctactgtaccctttacccaaatagtgaacattgtactcaatgggtaatttttcaaccctcgccctcctcccaacctccccycttcaggagtccccactgtctattatttctatctttgtgttcatgtttacccattgtttatctcccacttatgagtgaaaacacgtggtatttgatttcctgagttagttcactaaggatactgccctccaattccatccatattgctgcaaaggacatgatttcatttgttatggctgccgaataaattctattgagct

SEQ ID NO: 69 - DNA, 401 bp, Homo sapiens
tcctatttggaaaatctggaggcaggctgtctgtggctggtgccccaccaagtagttgaggacccaggctttttccctctttcccaagaggacctcccagtctaagatggttctggatcagtggctcttcatcagggataatttcccccctaaaggacagttgacaatgtctggagacatttttattgtcataatggggaygaggtgcaatggcatcaaaaaagtagaggccaggggtgctgctaaatgtcctacaacccctggcaacaaggaattatctaagcccaaaatgggaacggtgtggaggttgagaaatcccgttctcactccatctatcacacctgtgatccaagaagcaggtcagcatacagaagaaaggggcaaaaggcatatgccagctg

SEQ ID NO: 70 - DNA, 401 bp, Homo sapiens
cagtctaagatggttctggatcagtggctcttcatcagggataatttcccccctaaaggacagttgacaatgtctggagacatttttattgtcataatggggacgaggtgcaatggcatcaaaaaagtagaggccaggggtgctgctaaatgtcctacaacccctggcaacaaggaattatctaagcccaaaatgggaacrgtgtggaggttgagaaatcccgttctcactccatctatcacacctgtgatccaagaagcaggtcagcatacagaagaaaggggcaaaaggcatatgccagctgacttaagaaaagtttctagaagctgccacacaattccacttatattccattcaccaaaacttaatcacatggccacacataattggaaaatatagtc

SEQ ID NO: 71 - DNA, 401 bp, Homo sapiens
tcttcaatttttccttcatctccaagctttcatctgggatcttctctttacccaaagaatatctgtcagaatttcctttagtgcaggtctcttggtggcagcttctttcagtttttatttcactgactttattttaccatgattactgaaaaatatttttactaggtgtagaattctagattggccaaattgaaaatatcrttctactattttcttatttctgttatcatttttgagaagtctgctgtcattctttttgttcttggagatgagggtctcacagtgttgcccaggttggagtgccatggctagtcacaggtgcaatcatagctcactgcagcctcgaactcctggcctcaagcaatcctcccacccgcctcagcctcttgagtagcttggac

SEQ ID NO: 72 - DNA, 401 bp, Homo sapiens
atatttttaattagagtgtttctagaggttctctatcaaatgtggacaattactttttatagcttccaatttccaatccttgccaacaaatttcaagtttggcctttactttctttgtttgtttttgtttttgtttttgagacggagtcttgctctgttgcccaggctagagtgcaatggcacaatctctcctcaccgcargcttcacctcccgggttcaagcgattccattcccctgcctcagcttcctgagtaactgggattacaggtgcctgccaccacacctggctaatttttgtatttttagtagaggtggggtttcaccatactgaccaggttggtctcgaactcctgacctcaggtgatccgcccacctcggcctcccaaagtgctgggattac

SEQ ID NO: 73 - DNA, 401 bp, Homo sapiens
gtgcagcccttcggggccccaactctgagtgccaagtagtttaccagggtctccaccatggaaaagaaaggaagcagtagattcgaacttatgggcacctagccacccacagttttcaaaaacatggctcatctctctgtcttctctttagatgagccagtgccccctgggcaaaagtagccctaaatgccaagcttgccyctctagagcctcattgtctcctagatcttggcttgcttcctcactatcttattagcttttgatattttagggaattttttttaaatgttttctctgaatttgttagttgttttcagagggaaggttggcccatattagctagtctgcctctattagaagcagaagtctgatatggactttggatagaaatgaggatcaga

SEQ ID NO: 74 - DNA, 401 bp, Homo sapiens
attgtctcctagatcttggcttgcttcctcactatcttattagcttttgatattttagggaattttttttaaatgttttctctgaatttgttagttgttttcagagggaaggttggcccatattagctagtctgcctctattagaagcagaagtctgatatggactttggatagaaatgaggatcagaagtttttaagaayagagggacatggccaggcacggtggctcatgcctgtaatcccaacactttgggaggccaaggcgggtggatcacgaggtcaggagattgaaaccatccttgctaacatggtgaaaccccgtctctactaaaaatacaaaaaataaaaataaaaaatttagtcgggcatggtggcaggcatctgtagtcccagctactcgg

SEQ ID NO: 75 - DNA, 401 bp, Homo sapiens
agggcatccatagccaggactcttcgtctgtggtctgctgtactccgattccttatgatccccactcctactcctcactcagccctgttcctttacctcttatccaaacacttccataagatttcctatcagtcactgacccctcagtctagtatagctccaagggactagattctcctatctctggttctagagaaaaastttatccttcctaaaatgagttttaggctctgaggcacaggagacggtgagactttgtatagtgcttatgacagtgcttacttgagggaattcttccctggattcctgagattcctgagtattactgccttcttcacagaagcccaaccctgattctttcagatggacttatccctattcctcacatccaagcagtatca

SEQ ID NO: 76 - DNA, 401 bp, Homo sapiens
aaaataaatttagatgcatcaagctggcagatggagatgggaaaggggcctccccaagaagtcaagttctgctaaagctgcagtgcctaataaggtattatcttgcctctctgatcatttaagttagaaaagggaggcaataacaagaggaagttgtatggtttctcagtgtttatatctgagtgaaaagaaagaaccaamcagagtcatgaaagtgacatccttgggaggcagtgatttctcccctgctgttgaaagtggattttatttatttatttatttttgagatggagtcccactctgtcacccaggctggagtacagtggcgcgatctcggctcattgcaacctccacctcctgggttcaagcaattctcttgcctcagcctcccaagtagctgg

SEQ ID NO: 77 - DNA, 401 bp, Homo sapiens
tttgggaggccgaggcgggcagatcacgaggtcaggagatcgagaccatcctggctaacacggtgaaatcccgtctctactaaaaatacaaaaaattagccgggcgtggtggcgggtgcctgtggtcacagctactcgggaggctgaggcaggagaatggcgtgaatccaggaggcggagcttgcagtcagccgagatcgygccactgcactccagcctgggagacagagcaagactccgtctcaaaaaaaaaaaaatgcaaatccagtaggttaagaacacactagaagtcaagagcatgacacacacctgtagtcccagatactgaagaggctaaggtgggaggatggcttgagctcaggagatagaagctggagtgaattatgatcacacctgtgaat

SEQ ID NO: 78 - DNA, 401 bp, Homo sapiens
catcacaaatggaagggatctgagtccccacctcattgctcgggattgactgtcccacacatggtaagactgattgaaaactaagcctctcttgtgctggctactgaggctttggagttgcttgttaccttaagtagtactcattcctgtattacagaaatgcaataaagatctcacaaagagaatctagtagacaatacrttattggctgagtgtttgtgtaccacctccaaattcatatgttaaagcaccaaccccaagtgtaatggcagcaggagacggggcctttgagaggtgattagggtcatatgaggccacaagggtgaggccttcatgattagattaatgtctttataagaagaagaagagataccagagcattcatgagctctctcactccc

SEQ ID NO: 79 - DNA, 401 bp, Homo sapiens
caataaagatctcacaaagagaatctagtagacaatacgttattggctgagtgtttgtgtaccacctccaaattcatatgttaaagcaccaaccccaagtgtaatggcagcaggagacggggcctttgagaggtgattagggtcatatgaggccacaagggtgaggccttcatgattagattaatgtctttataagaagargaagagataccagagcattcatgagctctctcactccctcactcactttctccctctttctctcactcttcctttgtctccgctatgtgaggacacagcaagaaggtggccatctgcaaaccaagaagagagccctcaccagaaactgaaattgccagcaccttgatcttggacatcccagcctccagaatggtgagaaa

SEQ ID NO: 80 - DNA, 401 bp, Homo sapiens
ccgagtctgtagtgttttgtaatgacaaaccaagcagactaagacatgatgtatatgaaatagcagagaaggtcttcataactaagactgagaactagaagctattttttaaaaaaaaagataggcatattttgactgttaaaaattacaaggaaaacaagaaaattttgtacagtaaaagattccataaacaaactatakttgtcccttagtctacgcaggggattggtttcaggaccccctcttataccaaaagtcagtcctgcagaactggcatatatgaaaagttggccctttgtatatggaatttttgcattccataagtactgtattttcaatccacttttggttgaaaaaaatctacatataagtggagccatgcagttaaaacccatattatt

SEQ ID NO: 81 - DNA, 401 bp, Homo sapiens
gcgccactgcactctagcttgggtgacagagcaagactccatctcaaaaaaacaaacgaacaaaacaaaacaaaacaaaaactgtatgccctttggcctaacaattccacttctgggaatccatcttataaaaataaaattgccagtacatgaggatatatgcgtgagggtgtttcttacaggattgttcatagggggggraaatgaaaacaaatgaaattaccattaatagagacatagctaaataaatgtgggtatacccacaccatgaaatattatgccattatttaaaagaatgaataagataacaaatggcttaaagaagtcttcagcaaacatcgttgagtaagaaaagcaaacgacagtcaaatgttcataactcaatccattttaataagtta

SEQ ID NO: 82 - DNA, 401 bp, Homo sapiens
ggccactcgtcagctggtaatgtcagccatgtggtggtggcaggggcacaaaagatgcctgagggactccagctctgagggccacaatgctgtccctcctggaggcgtggtcttcctatggtgactgcccacttggttccatccttcccttaccccctcaaaatccgtgtcacataaagcccaccccagcagacctaattyttgttaaagtgccattctgtaagggcaccccctctggccattattccaccacgactaccacgggactcactttcctgtctgtgacaccctggagagggtctttaattccaggcattagtaaaacatccacagagagcaagtgacccgtgctccgccatgaaaccgacaagcagctcagctttctctctagttaacagaag

SEQ ID NO: 83 - DNA, 401 bp, Homo sapiens
agggcggggaacatcacacaccagggcttgtggggggtggagggtgggggactgggggagggatagcattaggagaaatacctaatgcaaatgataagttgacgggtgcagcaaaccaacatggcacatgtatacctatgtaacaaacctgcaggttgtgcacatgtaccctagaagttaaagtataataaaaattttttwaaaaaaaagaaattatggtttaggagtcatgacgatggaggctgcaagattctgaccctccctaaactgctcccaagatcagtgcctgacatatcttgcagacactgcagttgatggatcagctggcaccacctggatcaataaactggttcatcttatcttgtggcccccacccaggaactgactcagtacaagaggac

SEQ ID NO: 84 - DNA, 401 bp, Homo sapiens
gtttttttattattcaaatcagtctccccaaacatttggaaatcagagtttttaaggataatttggtgggtggaggaagtcctgtgagtcgagagtgctgactggttgggttggagatgaaatcatgggaagttgaaggtgtcctcttgtactgagtcagttcctgggtgggggccacaagataagatgaaccagtttatygatccaggtggtgccagctgatccatcaactgcagtgtctgcaagatatgtcaggcactgatcttgggagcagtttagggagggtcagaatcttgcagcctccatcgtcatgactcctaaaccataatttcttttttttaaaaaaatttttattatactttaacttctagggtacatgtgcacaacctgcaggtttgtta

SEQ ID NO: 85 - DNA, 401 bp, Homo sapiens
aaaactttccaaaatttctgtcccaatgacctggtctaggctgcagggccctcaggactgggtttccctgaattgagatagtgcagctactctactgctctgaaaaacaagacattttcaacttaatatccttggatatctaacctcccacaaacataaatttaatagctgcaaaaatatgattgtatatatactcatctstgtgcaatttccccccctttattgttttttgatttgctctttattcctccttttttcccttcctcttgtaacagctggtctgtgtcttgcctgctgcctagacagagctgacttatcaagacaggggaattgcaatagagaaagagtaattcacacagaaagcgggctgtggaggagattggagtttttttattattcaa

SEQ ID NO: 86 - DNA, 401 bp, Homo sapiens
gacaggctcaggagactgggcctgatgggaggtcagggagatgtgtgactttccttgtgatcacagcaacagaagggtttttccagcttgcaaatgacctctcaaaataacctgaatttccacccagggctggtgggaatgtaagctgatacaaacttcggggaaataatgaggtggaagatagtaactggcttaagaacrtaaagggcagaggagtggaggttgagactcagcgtcctaggattaagagaaacctgtcctggtcatcccactcctgaaagtctgtcctaaaggacttctacaccaaaatggtcatttcagatttacttatagtaacttaaaagtaaggtagagttggaaacaactcaaatatgcaaaaagtaagcaaatttgttaagcaa

SEQ ID NO: 87 - DNA, 401 bp, Homo sapiens
cagtctatcattgttggatatttgggttggctccaagtctttgctattgtgaataatgccgcaataaacatacatgtgcatgtgtctttatagcagcatgatttataatcatttgggtatatacccagtaataggatggctgggtcatatggaatttctagttctagatccctgaggaatcgccacactgacttccacaakggttgaactagtttacagtcccaccaacagtgtaaaagtgttcctatttctccacatcttcttcagcacctgttgtttcctgactttttaatgatcgccattctaactggtgtgagatggtatctcattgtggttttgatttgcatttctttgatggccagtcatgatgagcattttttcatgcagtttttggctgcata

SEQ ID NO: 88 - DNA, 401 bp, Homo sapiens
tttgcatttctttgatggccagtcatgatgagcattttttcatgcagtttttggctgcataaatgtcttcttttgagaagtgtctgttcatatcctttgcccactttttgatggggttgtttgtttttttcttgtaaatttgtttgagttcattgtagattctgtatattagccctttgtcagatgagtagattgcaaaawttttctcccattctgtgggttgcctgttcactctgatggtagtttcttttgctgtgcagaagctctttagtttaattagatcccacttgtcaattttggcttttgttgccattgcttttggtgttttaggcatgaagtccttgcccatgcctgtgtcctgaatggtattgcctaggttttcttctagggtttttatggtt

SEQ ID NO: 89 - DNA, 401 bp, Homo sapiens
taaggaagggatccagtttcagctttctacatatggctagccagttttcccatcaccatttattaaatagggaatcctttccccattgcttgtttttgtcaggtttgtcaaagatcagatagttgtagatatgcggcattatttctgagggctctgttctgttccattggtctatatctctgttttggtaccagtaccatrctgttttgcttactgtagccttgtagtatagtttgaagtcaggtagcatgatgcctccagctttgttcttttggcttaggattgacttggcaatctgggctcttttttggttccataagaactttaaagtagttttttccaattctgtgaagaaagtcattggcagcttgatggggatggcattgaatctgtaaattacc

SEQ ID NO: 90 - DNA, 401 bp, Homo sapiens
gagagagggcatccctgtcttgtgccagttttcaaagggaatgcttccagtttttgtccattgagtatgatattggctgtgggtttgtcatagatagctcttattttgagatacgtcccatcaatacccaatttattgagagattttagcatgaaggttgttgaattttgtcaaaggccttttctgcatctattgagatawtcatgtggtttttgtctttggttctgtttatatgctggattatgtttattgatttgcgtatgctgaaccagccttgcatcccagggatgaagtccacttgattatggtggataagctttttgatgagctgctggattcggtttgccagtattttattgaggatttttgcatcaatgttcatcggggttattggtctaaaa

SEQ ID NO: 91 - DNA, 401 bp, Homo sapiens
agggcatccctgtcttgtgccagttttcaaagggaatgcttccagtttttgtccattgagtatgatattggctgtgggtttgtcatagatagctcttattttgagatacgtcccatcaatacccaatttattgagagattttagcatgaaggttgttgaattttgtcaaaggccttttctgcatctattgagatattcatrtggtttttgtctttggttctgtttatatgctggattatgtttattgatttgcgtatgctgaaccagccttgcatcccagggatgaagtccacttgattatggtggataagctttttgatgagctgctggattcggtttgccagtattttattgaggatttttgcatcaatgttcatcggggttattggtctaaaattctc

SEQ ID NO: 92 - DNA, 401 bp, Homo sapiens
ggtatcaggatgatgctggcctcataaaatgagttagggaggattccctctttttctattgattggaatagtttcagaaggaatggtaccagctcctccttgtgcctctggtagaattcggctgtgaattcatctggtcttggactttttttggttggtaagctattaattattgcctcaatttcagagcctgttattggyctattcagagattcaacttcttcctggtttagtcttgggagggagtatgtgtcgaggaatttatccatttcttctagattttctagtttatttgcatagaggtgtttatattattctctgatggtagtttgtatttctgtgggattggtggtgatatccccttcgtcattgtttattgcgtctatttgattcttctctct

SEQ ID NO: 93 - DNA, 401 bp, Homo sapiens
cactgtggtctgagagacagtttgttataatttctgttcttttacattagctgaggagtgctttacttccaactatgtggtcaattttggaataggtgtggtgtggtgctgaaaagaatgtatattctgttgatttggggtggagagttctgtagatgtctattaggtccacttggtgcagagctgagttcaattcctggrtatccttgttaactttctgtctcattgatccatctaatgttgacagtggggtgttaaagtctcccattattattgtgtgggagtctaagtctctttgtaggtcactcaggacttgctttatgaatctgggtgctcatgtattgggtgcatatatatttaggatagttagctcttcttgttgaattgatccctttaccatt

SEQ ID NO: 94 - DNA, 401 bp, Homo sapiens
cctggatatccttgttaactttctgtctcattgatccatctaatgttgacagtggggtgttaaagtctcccattattattgtgtgggagtctaagtctctttgtaggtcactcaggacttgctttatgaatctgggtgctcatgtattgggtgcatatatatttaggatagttagctcttcttgttgaattgatccctttmccattatgtaatggccttctttgtctcttttgatcttagttgatttaaagtctgttttttcagagactagggttgcaacacctgcctttttttgttttccacttgcttggtagatcttccttcatccctttattttgagcctatgtgtgtctctgcatgtgagatgggtttcctgaatacagcacactgatgggtcttga

SEQ ID NO: 95 - DNA, 401 bp, Homo sapiens
ttcccatctttgtggttttatctacctttggtctttgatgatggtgacgtacagatgggtttttggtgtggatgtcctttctgtttgttaattttccttctaacagtcaggaccctcagctgcaggtctgttggagtttgctggacgtccactccagaccctgtttgcctgggtatcagcagcggtggctgcagaacagcrgatattggagaaccgcaaatgctgctgcctgatccttcctctggaagttttgtctcagaggtgcacccagtcatgtgaagtgtcagtccgcccctactggggggtgcctcccagttaggctactcaggggtcagggacccacttgaggaggcagtctgcctgttctcagatctcaagctgcgtgctgggagaacgactac

SEQ ID NO: 96 - DNA, 401 bp, Homo sapiens
ggatgtcctttctgtttgttaattttccttctaacagtcaggaccctcagctgcaggtctgttggagtttgctggacgtccactccagaccctgtttgcctgggtatcagcagcggtggctgcagaacagcagatattggagaaccgcaaatgctgctgcctgatccttcctctggaagttttgtctcagaggtgcacccrgtcatgtgaagtgtcagtccgcccctactggggggtgcctcccagttaggctactcaggggtcagggacccacttgaggaggcagtctgcctgttctcagatctcaagctgcgtgctgggagaacgactactctcttcaaagctgtcagacagggacatttaagtctgcagaggttactgctgacttttgtttgtatgtg

SEQ ID NO: 97 - DNA, 401 bp, Homo sapiens
tgggcataggaccctccaagccatgtgcgggatatcatctcctggtgtgccatttgttaagcccgttggaaaagcacagtattagggtgggagtgacccgattttccaggtgccatctgtcacccctttctttgactaggaaagggaattccctgaccccttgcgcttcccaggtgaggtgatgcctcgccctgcttcggytcatgcatggtgtgctgcacccactgtccacccactgtccagcactccccagtgagatgaacccggtacctcagttggaaatgcagaaatcacccgtcttctgcatcgcttacactgggagctgtagactggagctgctgctattcggccatcttggctccacctccacagagttttaatgtcatgggaaaattctattg

SEQ ID NO: 98 - DNA, 401 bp, Homo sapiens
gatattggagcttcttcctcctgtgctgtcttctctgcctggggaaatttgtgtccctcagctaggccacccaggggaatcacatgatgatcctgaacattccatcccatcaggctgtgcacaggactggctgcatctgagatcaaacgatgttcaaagaaagttcagactccagcactccagttagggcaagcctcttaraaggtgagcgtcttgatacctgtttggaaggagggaacactggttgcaggaaattcaggtgagcatttttgaaagaggtggtgtatagactccaaagcatcttcctcctactttcttaaatttttttgttttgtgtgtttgggtttttgtgttttgaatggggggttgtttttcggtttttttgagatggagtctcgctt

SEQ ID NO: 99 - DNA, 401 bp, Homo sapiens
tgctaggattacaggcgtgagccaccacatccggccctgtgtgtttgttttttgagacagggtctcactatgttgtccaggctggtctcgaactcctgagctcaagcaatcctcctcccttggccttcccagtaattggcttttaaacatttgatggagggtgctggtgagcaaaacttgacagttatgaaacaggaaacsagcaggaaaaggacctgcccaaacttggaggagaaggtagtgccagggaagaagggatggcactgagcttagtgtgcaacccagaatttgtgacaattgcacaacacaccctcttgtcattcctgtcagttttattcattcccctccctcccttccattcctttcccagtctttaatccccctgttccttcccttctcct

SEQ ID NO: 100 - DNA, 401 bp, Homo sapiens
ttttttgagacagggtctcactatgttgtccaggctggtctcgaactcctgagctcaagcaatcctcctcccttggccttcccagtaattggcttttaaacatttgatggagggtgctggtgagcaaaacttgacagttatgaaacaggaaaccagcaggaaaaggacctgcccaaacttggaggagaaggtagtgccagrgaagaagggatggcactgagcttagtgtgcaacccagaatttgtgacaattgcacaacacaccctcttgtcattcctgtcagttttattcattcccctccctcccttccattcctttcccagtctttaatccccctgttccttcccttctcctactcccttaatcacttacagtcccactcccaggaggaaggggtacag

SEQ ID NO: 101 - DNA, 401 bp, Homo sapiens
ctttccccacactgtcctactgcctctcctcaccttctgccccaccctcactctgcatgactgaatgacttaattcaaattgcatttatttgctcatttctaatcacatcctaggatctcatgctctcttgatggtaaataaaagtgaacagaaaatgtcttctaatgacctggtaaatcaagtttatacctttcccaaakcagggtcaatttgctctaaatctagtcaagtcagaacccagcattagtaggtggcctgaccacatggccccttcccccagtctcttaaggcatagtgagtcactcacagaggccagcttagcccttcatgagtcagtatctcctgtttatgtacagagtcattactttctctataggaaaaaaaggaggtggtggtggag

SEQ ID NO: 102 - DNA, 401 bp, Homo sapiens
agtgagtcactcacagaggccagcttagcccttcatgagtcagtatctcctgtttatgtacagagtcattactttctctataggaaaaaaaggaggtggtggtggaggtgcttgtctatagatggactgggcattgtaatttgaaaagcattgagaaaagtccctatctccaaagacattatcagccttacataatatctraaaacctatcatttgagacatttctcagagcaatgatcctttataaaatatgggatgaggccagaagagccattttactatgtgacagtcagtacaactcagtggctaggaatcaggatctggatcgagttccacttctggtatggctgagtaaacgcttactgtactgagcttcctgccaataacaactataaactcag

SEQ ID NO: 103 - DNA, 401 bp, Homo sapiens
ctggcctgaagaactggcagacagagtttgagggcaaccccagctgctagaaagtgagggagaatcctcaaagggagagagccagagaaagaaaatcccaaattccatgtataaagtctgccccaatttttgcctgacccccaaacacccatgtacagcagagattgtaagcaccctatctaagggtgaaagaacaaaaarcagatatgaggggccactcagaaaaaaacagaatttgcagtttgaaccataacaagttaatttctcattaaaatgaaaaattcaaaactctttgaaagaatacaactgaatccagtctccacaatatgacattcacaatatccagagtacaatccaaaatttcttgacatattgagagacaggaagatgtgatgtattct

SEQ ID NO: 104 - DNA, 401 bp, Homo sapiens
tataatgattaaaacatagttagtatcatcacctaaatagagacgcctaaaattataatcaaatggaaattctataactaaaaaatacagtatctaattttaaaaaattaactggatggggcttaacataattttttaaaacagatgaaagagtaggtgaatctaaagagagatcaacagaaaatatccaatctgaaagayagagataaaaatgatagtacctaaagggtggtattgaaaggtctaacatacctacaattgaactcctagaagaacagaggacatggggtagaaaaaaattatttgaataaataatggccaaaaagtccccaaatttggcaaaagacataaatttacaaatctaagaagctcagcaaatcccaatcaagttaaataaaaga

SEQ ID NO: 105 - DNA, 401 bp, Homo sapiens
acattagtacactactattaattaagtgccagactttattcagatttctttgtttcttcacaaataacccctttttcctgtttcaggagccaatctagggtaccatattgcatttagtgccttttagaaattattatttggacatgttttagaattccagtttaatttacatattaatgttttttctatatattattgtaktttttacccaatgtccgctctaagaatcacgatatacattcttaactttttataatctgatttgagtttacattgtcccactttgcataaaatatgcaaaccttgcaatcgtataggtcctcttatacctgcccccagcattcctttgtgttatgattaccatatgacatttatatacattatgcatcctacaatgcaag

SEQ ID NO: 106 - DNA, 401 bp, Homo sapiens
taaactcaaatcagattataaaaagttaagaatgtatatcgtgattcttagagcggacattgggtaaaaactacaataatatatagaaaaaacattaatatgtaaattaaactggaattctaaaacatgtccaaataataatttctaaaaggcactaaatgcaatatggtaccctagattggctcctgaaacaggaaaaargggttatttgtgaagaaacaaagaaatctgaataaagtctggcacttaattaatagtagtgtactaatgttagtttcttagttgtgatgaatgtactgtgataatgtaagatgttaacttcagcagaaactgggtgagaggtatacaggaactctctgtactatttttgcaacttttatgtaaatctaaaatttaaacaa

SEQ ID NO: 107 - DNA, 401 bp, Homo sapiens
acaccattttatttgttttcttttttgcccctctgatttttgttcttctgttacttctttcttgtctttttttttttttttacagaagctctttattttgtttaaattttagatttacataaaagttgcaaaaatagtacagagagttcctgtatacctctcacccagtttctgctgaagttaacatcttacattatcacrgtacattcatcacaactaagaaactaacattagtacactactattaattaagtgccagactttattcagatttctttgtttcttcacaaataacccctttttcctgtttcaggagccaatctagggtaccatattgcatttagtgccttttagaaattattatttggacatgttttagaattccagtttaatttacatat

SEQ ID NO: 108 - DNA, 401 bp, Homo sapiens
ttcctacaaggattatagatgtctttactgtgtcatgcaggttgtcagagcagtgcaggaaagccagcagttacaggtctcacccagctcccacataatccgaagggctggtctcactttcactgtgccccctgccccccgcctgccaatagcactgagtctgtttccaggcaatgggcgagcagggctgagaacttgccscacgatacctgcctcccagctgcagtagcaagtatgtctttccttcttcccctgcctgtggagtctgcacaccagattcatgccctcccctgagttctgaccaggagagttctccaccagttcaaattgtttcaaagttcagctgaagatgtccttctctctgtggccttttcccagtgcctctggctgcccttctgaag

SEQ ID NO: 109 - DNA, 401 bp, Homo sapiens
cctggaaacagactcagtgctattggcaggcggggggcagggggcacagtgaaagtgagaccagcccttcggattatgtgggagctgggtgagacctgtaactgctggctttcctgcactgctctgacaacctgcatgacacagtaaagacatctataatccttgtaggaacatagctccattgacctgggagcctcatcyccatcccccacagcagatgcagcaagacctgcccaaggagagtctgtgcttagacatgcctagccctgcccccacacaatggtccttccctatccaccctggtaattgaagacaaagggcatatactcttgggagttctagggccctgcccaccacctgttcttccccatactactgcagctgatgctctctggaaagcg

SEQ ID NO: 110 - DNA, 401 bp, Homo sapiens
ccttctcatttgggtaggttctgtcagagggaaggtctacggctgaagtctgttgttcagattttttttgtcccacagggtgttcccttgatgtagaactctctcccttttcctatggatgtggcttcctgagagccgagctgtagtgattgttttctctctttaggaccttgccacccagtaagtctaccaggctctggkctggtattgggagttttctgcacagagtcctgtgatgtgaaccgtctgtgggtctctcagctgtggataccagcacctgttctagtggaggtggcagggaggtgaaatggactctgtgagggttcttagctttggtggtttaatgtagtatttttgtgctggttggcctcctgccaggaggtggcgctttccagagagca

SEQ ID NO: 111 - DNA, 401 bp, Homo sapiens
tttgtcttaactttacataacctgttgacaacgtgcataggtatgatctttttgtgatgaatttcccaggtgttttttgtgtttcttgtatttggatgtctagatctctagcaagtctggggaagttttccttgattattcccccaaataggttttccaaactttcaaatgtctgttcttcctcaggaacactgattattytaggtttggtcatttaatagaatcccagactgcttggaggctttgttcatattttcttatcttttttctttgtctttgttagactgggttaatttgaagaccttgtcttcaagctctgaatttctttcttctacttgttcgattctattgctgagactttccagagcattttgcatttctataagtgtgtccattgtttc

SEQ ID NO: 112 - DNA, 401 bp, Homo sapiens
tctgtatatatctgttaattccatttgttctagggtatagtttaattcaattttttttgttgcctttctgccttgatgacctctctaatcctgttagtgtagtattgaagtcccccactattattgtgttgctgtctatctcactttttaggtctagtaattgttttataaatttgggagctccagtgttagatgcatatrtatttaggattgtgatattttcctgttggacaaggccttttatcattaaataatgtccttctgtctttttaaactgctgtcactttaaagtttgtttgaatagctactcctgctcacttttggtgtcaatttgcatgaaatgtctttttccacccctttaccttaagtttatgtgagtccttatgtgttaggtgagcctc

SEQ ID NO: 113 - DNA, 401 bp, Homo sapiens
ttccactgtggtctgagagagtgcttgatatgatttccattttcttaactttattgaggctcattttgtggcctatcatatggtctatcttggagaaggttccatgtgctgttgaatagaatgtatagtctgcagttgttagatggaatgttctgtatatatctgttaattccatttgttctagggtatagtttaattcawttttttttgttgcctttctgccttgatgacctctctaatcctgttagtgtagtattgaagtcccccactattattgtgttgctgtctatctcactttttaggtctagtaattgttttataaatttgggagctccagtgttagatgcatatatatttaggattgtgatattttcctgttggacaaggccttttatcattaa

SEQ ID NO: 114 - DNA, 401 bp, Homo sapiens
cctttgctgtatcccagaggttttgataggttgtgtcactattgtcgtttagttcaaagaactttttaattttcatcttgatttcacttctgacccaatgatcattcaggaacaggttatttaatttccatgtatttgcatggttttgaaggttccttttggagttgatttccagttttattccactgtggtctgagagartgcttgatatgatttccattttcttaactttattgaggctcattttgtggcctatcatatggtctatcttggagaaggttccatgtgctgttgaatagaatgtatagtctgcagttgttagatggaatgttctgtatatatctgttaattccatttgttctagggtatagtttaattcaattttttttgttgcctttctg

SEQ ID NO: 115 - DNA, 401 bp, Homo sapiens
tgctctaatcttggttatttcttttcttctgttgggtttgggtttggtttgttcttgtttctctagttcttcgaggtgtgaacttagattatctgtttgtgctctttcagaccttttgatgtgggcatttagggctatgaactttcctcttagcaccgcctttgctgtatcccagaggttttgataggttgtgtcactatygtcgtttagttcaaagaactttttaattttcatcttgatttcacttctgacccaatgatcattcaggaacaggttatttaatttccatgtatttgcatggttttgaaggttccttttggagttgatttccagttttattccactgtggtctgagagagtgcttgatatgatttccattttcttaactttattgaggctca

SEQ ID NO: 116 - DNA, 401 bp, Homo sapiens
cattggggatattggtctgtagttttcttttttggttttgtcctttcctggttgtggtgttaggatgatgctggcttcatagaatgatttaggaagggttccttctttctccatcttgtggaatagtgtcaataggattggtaccaattcttttttgaatgtctggtagaattctgctgtgaatccgtctggtcctggccwttttttgttggtaatttttaaattaccatctcaatctcactgcttgatattggtctgtgcagggtgtttaattcttcctgatttaagctaggagagttgtatctttctaagaatttatccatctcttctaggttttctagtttatgcacataaagatgttcatagtatccttgaatgatcttttgtatttctgtggtttc

SEQ ID NO: 117 - DNA, 401 bp, Homo sapiens
tattgacttgcatatgttaaaccatccctacatccctgctatgaaatccacttgatcatggatcatggagtattatctttttttttgttgttgttagacagagtcccactctgtcacctaggctagggtgcagtggcactatttcggctcactgcaacctccgcctcccaggttcaagtgattctcctgtctcagcttccygagtagctgggattacaggtgtgcgccacaatgcctggctaatttttgtatttttagtagagacagggttttgccatgttagccaggctagccttgggtgatccacccacctgggtctcccaaagtgctgggattacaagcatgagctactgagcccggccggattatctttttgatatgttgttggctttggttagcta

SEQ ID NO: 118 - DNA, 401 bp, Homo sapiens
attgctgagagttttaattataaagggatactggattttgtcaaacgctttttctgcatctattgagatgattatgtgatttttgtttttaattctgtttatgtgttgtatcacatttattgacttgcatatgttaaaccatccctacatccctgctatgaaatccacttgatcatggatcatggagtattatcttttttkttgttgttgttagacagagtcccactctgtcacctaggctagggtgcagtggcactatttcggctcactgcaacctccgcctcccaggttcaagtgattctcctgtctcagcttcctgagtagctgggattacaggtgtgcgccacaatgcctggctaatttttgtatttttagtagagacagggttttgccatgttagc

SEQ ID NO: 119 - DNA, 401 bp, Homo sapiens
agggaggattccctctttctctattgattggaatagtttcagaaggaatggtaccacctcctccttgtacctctggtagaatttgaaactgctgaattcttttatcagttctaggagctttctagaggaattgttagggttttctaggtaaacaatcatatcatcagcaaagtgacagtttgacttcctccttactgattkggatgccctttatttctttctcttgtctgactgctctggctaggacttccagtaatatgttgaagaggagtggtgagagtgggcgtccttatcttgtttcagttctcagagggaatgctttcaactttttcccattcagtattatattggctgtgggcttgtcatagatggcctttattacattgaggtatgtctcttgt

SEQ ID NO: 120 - DNA, 401 bp, Homo sapiens
taatttacagattcaatgccatccccatcaagctaccaatgactttcttcacagaattggaaaaaactactttaaagttcatatggaaccaaaaaaagggccctcattgccaagtcaatcctaagccaaaagaacaaagctggaggcatcacactacctgacttcaaactatactacaaggctacagtaaccaaaacagcwtggtactggtaccaaaacagagatatagaccaatggaacagaacagggccctcagaaataatgtcgcatatctacaactgtctgatctttgacaaacctgacaaaaacaagcaatggggaaaggattccctatttaataaatggtgatgggaaaaccggctagccatatgtagaaagctgaaactggatcccttccttac

SEQ ID NO: 121 - DNA, 401 bp, Homo sapiens
aactactttaaagttcatatggaaccaaaaaaagggccctcattgccaagtcaatcctaagccaaaagaacaaagctggaggcatcacactacctgacttcaaactatactacaaggctacagtaaccaaaacagcttggtactggtaccaaaacagagatatagaccaatggaacagaacagggccctcagaaataatgycgcatatctacaactgtctgatctttgacaaacctgacaaaaacaagcaatggggaaaggattccctatttaataaatggtgatgggaaaaccggctagccatatgtagaaagctgaaactggatcccttccttacaccttatgcaaaaattaattcaagatggattaaagacataaaagttagacctaaaaccataaaa

SEQ ID NO: 122 - DNA, 401 bp, Homo sapiens
cagtgtgtgatgttccccttcctgtgtccaggtgttctcattgttcaattcccacctatcagtgagaacatgcagtgtttggttttttgtccttgtgagagtttgctgagaatgatggtttccacctttatccacgtccctacaaaggacctgaactcatcctttttatggctgcatagtattccatggtgtatatgtgcyacattttcttaatccagtctgtcattgctggacatttgggttggttccaagtctttgctattgtgaatagtgctgcaataaacatacgtgtgcatgtgtctttatagcagcatgatttataatcatttgggtatatacccagtaatgggatggctgggtcaaatggtgtttctagctctagatccttgagaaatcgccac

SEQ ID NO: 123 - DNA, 401 bp, Homo sapiens
gcacttttctctttatccctaaatatttcaggttctaactttctcttacataataactgtatagttatcaaatttacagaatttaacatgtatgcattattacataatttgcattccatatacaaaatctgccaactgtcccatggtgttctttagaggaatttttacttcttatctcaagatccaatccaggatcaaatygatcccatgttgcatttagttatgtagtctctttagtcttctttaatctagaacatttctcgagcctttatctctcatgaattaacattttggaagagtacaagctagttgtcttgcagaatgcccttcaactgggtttgtctcgtccttcctcatgattagtgtatgttacctgtcccctctggaataccataaaagtg

SEQ ID NO: 124 - DNA, 401 bp, Homo sapiens
tctagattaaagaagactaaagagactacataactaaatgcaacatgggatcaatttgatcctggattggatcttgagataagaagtaaaaattcctctaaagaacaccatgggacagttggcagattttgtatatggaatgcaaattatgtaataatgcatacatgttaaattctgtaaatttgataactatacagttaytatgtaagagaaagttagaacctgaaatatttagggataaagagaaaagtgcatgatatctgtgactcatcaaagaatcataaagtgttagaggttgaagggtccctagagagtcagtaggccaaaccctactttcttgaaatgatgtgaccaaaatcctgagaaattgaattatctgcctaagaccacacatccagcca

SEQ ID NO: 125 - DNA, 401 bp, Homo sapiens
tctatgtgcctatttttgtgctagcactatgctgtttttgtgactatggccttatagtatagtttgaaatcaggtaatgtgatgcttctagatttgttctttttgcttagtcttgctttggctatgcgggctcttttttggttctatatgaatttcagaattgctttttctaattctgtgaagaatgatggtagtatcgtwgtgaattgtgttgaatttgtagattgcttttggcagtatggtcattttcacaatattgattgtatccatgcatgagcatgggatgtgtttccatttgtttgtgtcatctatggtttctttcagcaatgttttgtagttttccttgtagaggtctttcacctccttggttaggtagattcgtaagtattttattttatttt

SEQ ID NO: 126 - DNA, 401 bp, Homo sapiens
cacacctatcttctattaattaatgtgcatcctctgaggacctgggcaagtcttcattgttggtttagttgttttgagaagcaacacaattgctttttaaaatacaattatataatttagatatctatatattttctgcattctcagtgctccctaaatcctcaccccaacttcttctcactccccagctgtgtttccttkctggccttgaacttctcagctgctgaacttttttcctcctctcagtacttcaacaatttcccttaaagggacgcttcagatcaatgaaacaaagagagtttataaatagcctgccaagcagagggaaacatagcataagatatttcaaattattggaaagaagtggattatttaataattggtactgggaaaactatttg

SEQ ID NO: 127 - DNA, 401 bp, Homo sapiens
ggaacttgagtgaggtcagtgtaacaccagcttatatcacacttagattgggaagaaggacagaatgcttggggttttctaagtcattctctatcagagtccatgaggccatgccctcttgctcattctggatggagatagtcattcttctccatgccttctcttctctggaaaacccggttattttcagcttcccatgcygttttttccctctaccctatccatcacatgggaaaggccatattgtgtcctggcaagagcacagtttctgcaccagatgggggtgaaactgggtcccagctttctcactaacaacaggatgactttgggcaaatacatattttatttcctgtgcaaaatgaggatgatgatggcctccttatggtattgctacaagaaag

SEQ ID NO: 128 - DNA, 401 bp, Homo sapiens
tcctgtgttctgttcctcctcctccctttggccgtgccttggctcaggctgtatcccttctcatgggactccttcaacttcttggtcactctctgcaagcacagcgacttttgaaaacaatgaaattgataatttctcttccctgcttaaaaccctttcatgacatttcattgttctcttcttaaagttccaactgtcacrcctgtaatcccagcatgttgggaggctgaggcgggcagatcacaaggtcaggagatcgagaccatcttggctaacacggtgaaacaccgtctctactaaaaatacaaaatattagctgggtgtggtggtgggtgcctgtagtctcagctacttgggaggctgaggcaggagaatggtgtgaagccaggaggcggagcttg

SEQ ID NO: 129 - DNA, 401 bp, Homo sapiens; identical to SEQ ID NO: 82

SEQ ID NO: 130 - DNA, 401 bp, Homo sapiens
tttggtgaaggcaaagtcctccttcttcattagcggtctcccatgtggggccacatcttccctcaccaggaacccagtgggcgcgctccagcccccctcagcttgccttttgcgtggtcattagagctagggcacacgtcatgctgattcacatatttttgccctttgtcatgtattgagaaaaagtaaggatgaatggayggtctttgattggcggcgctggtgacgcccgtcatggtcctgtttggaaggacccttttggaactaaagctggtgacgcagcgcgcagaggcatcgcccggctaagcttggccctggcagatgggtcgcaggaacaggtatgcttccttcgtgcagcctctggctcggggaacctgggagcctgctccaaactctggtgt

SEQ ID NO: 131 - DNA, 401 bp, Homo sapiens
ctccataagaaaaaaaaatggagaggtttgcaggttaaaagataaaagtagaaaaatggtgagtaaagagatctataaaaagttggagaatataactaaaggtataagtagaagtaccaggaaaccatttcccccaaagctacttctcctgtctctccaaaatgtgaaaggataaatacctttaagctggttggcaagttytttcatgtcaccggtgacatagccaagagctttggggaagcaccttctcccttgacatggcgaaacctggttttcacccctaaccatagtggggctgtccactctaaccatagtggggctgtcctccatggctatctcttcctctggctccttgagggaaggacagagcttataagtatccctaaggcagagccctccag

SEQ ID NO: 132 - DNA, 401 bp, Homo sapiens
gagccagggcgaatgtgagaagccagtgtctcttacctccagggcactgaggtcagaccaggagatatttggagctgtagttataggtatgaccagtttggctggaaagaatggattgtaatgtcctgtggctgaaaggtacattgacaacgccatgttgaaagggagggtgaagacggggaggtcccatttgctgagcayggaattcaatgcacttgagaaaattgggctaaaaaaagaacagagcaaaagaatgtgcatgtgacaaagcaggctgacacacggaatcgctatactctggaggggctttgtttcacctgccaacaggccccttacgtgttgtgttggcacatttgccacagaaagaaaacttgggataaatcctattatagaggcagtaa

SEQ ID NO: 133 - protein, 445 aa, Homo sapiens
MNGRSLIGGAGDARHGPVWKDPFGTKAGDAARRGIARLSLALADGSQEQEPEEEIAMEDSPTMVRVDSPTMVRGENQVSPCQGRRCFPKALGYVTGDMKELANQLKDKPVVLQFIDWILRGISQVVFVNNPVSGILILVGLLVQNPWWALTGWLGTVVSTLMALLLSQDRSLIASGLYGYNATLVGVLMAVFSDKGDYFWWLLLPVCAMSMTCPIFSSALNSMLSKWDLPVFTLPFNMALSMYLSATGHYNPFFPAKLVIPITTAPNISWSDLSALELLKSIPVGVGQIYGCDNPWTGGIFLGAILLSSPLMCLHAAIGSLLGIAAGLSLSAPFEDIYFGLWGFNSSLACIAMGGMFMALTWQTHLLALGCALFTAYLGVGMANFMAEVGLPACTWPFCLATLLFLIMTTKNSNIYKMPLSKVTYPEENRIFYLQAKKRMVESPL

SEQ ID NO: 134 - DNA, 4129 bp, Homo sapiens
acacagagcagagtggggctctgagtatataactgttaggtgcctccctccagcaccatctcctgagaagcactctcccttgtcgtggaggtgggcaaatctttatcagccactgccttctgctgccaggaagccagctagagtggtctttaaagaaaactgggcatctcctgctacttaaaatcaaaaactacctaaaataaagattataaaaaagtaaggatgaatggacggtctttgattggcggcgctggtgacgcccgtcatggtcctgtttggaaggacccttttggaactaaagctggtgacgcagcgcgcagaggcatcgcccggctaagcttggccctggcagatgggtcgcaggaacaggagccagaggaagagatagccatggaggacagccccactatggttagagtggacagccccactatggttaggggtgaaaaccaggtttcgccatgtcaagggagaaggtgcttccccaaagctcttggctatgtcaccggtgacatgaaagaacttgccaaccagcttaaagacaaacccgtggtgctccagttcattgactggattctccggggcatatcccaagtggtgttcgtcaacaaccccgtcagtggaatcctgattctggtaggacttcttgttcagaacccctggtgggctctcactggctggctgggaacagtggtctccactctgatggccctcttgctcagccaggacaggtcattaatagcatctgggctctatggctacaatgccaccctggtgggagtactcatggctgtcttttcggacaagggagactatttctggtggctgttactccctgtatgtgctatgtccatgacttgcccaattttctcaagtgcattgaattccatgctcagcaaatgggacctccccgtcttcaccctccctttcaacatggcgttgtcaatgtacctttcagccacaggacattacaatccattctttccagccaaactggtcatacctataactacagctccaaatatctcctggtctgacctcagtgccctggagttgttgaaatctataccagtgggagttggtcagatctatggctgtgataatccatggacagggggcattttcctgggagccatcctactctcctccccactcatgtgcctgcatgctgccataggatcattgctgggcatagcagcgggactcagtctttcagccccatttgaggacatctactttggactctggggtttcaacagctctctggcctgcattgcaatgggaggaatgttcatggcgctcacctggcaaacccacctcctggctcttggctgtgccctgttcacggcctatcttggagtcggcatggcaaactttatggctgaggttggattgccagcttgtacctggcccttctgtttggccacgctattgttcctcatcatgaccacaaaaaattccaacatctacaagatgcccctcagtaaagttacttatcctgaagaaaaccgcatcttctacctgcaagccaagaaaagaatggtggaaagccctttgtgagaacaagccccatttgcagccatggtcacgagtcatttctgcctgactgctccagctaacttccagggtctcagcaaactgctgtttttcacgagtatcaactttcatactgacgcgtctgtaatctgttcttatgctcattttgtattttcctttcaactccaggaatatccttgagcatatgagagtcacatccaggtgatgtgctctggtatggaatttgaaaccccaatggggccttggcactaagactggaatgtatataaagtcaaagtgctccaacagaaggaggaagtgaaaacaaactattagtatttattgatattcttggtgtttagctggctcgatgatgttaacagtattaaaaattaaaccccataaaccaactaagccttatggaattcacagtcacaaaatcgaagttaatccagaattctgtgataagcagcttggctttttttttaaatcaatgcaagttacacattatagccagaatctgtatcacagaggtgcaagctgacagcagagctcagtccccacttcctgcaaacaatggcctgcaccctatcccttgtgtgtgtgacattctctcatgggacaatgttggggtttttcagactgacaggactgcaagagggagaaaggaattttgtcaatcaaaattattctgtattgcaacttttctcagagattgcaaaggattttttaggtagagattatttttccttatgaaaaatgatctgttttaaatgagataaaataggagaagttcctggcttaacctgttcttacatattaaagaaaagttacttactgtatttatgaaatactcagcttaggcatttttactttaacccctaaattgattttgtaaatgccacaaatgcatagaattgttaccaacctccaaagggctctttaaaatcatattttttattcatttgaggatgtcttataaagactgaaggcaaaggtcagattgcttacgggtgttatttttataagttgttgaattccttaatttaaaaaagctcattattttttgcacactcacaatattctctctcagaaatcaatggcatttgaaccaccaaaaagaaataaagggctgagtgcggtggctcacgcctgtaatcccagcactttggggagcccaggcgggcagattgcttgaacccaggagttcaagaccagcctgggcagcatggtgaaaccctgtatctacaaaaaatacaaaaattagccaggcatggtggtgggtgcctgtagttccagctacttgggaggctgaggtgggaaaatgacttgagcccaggaggaggaggctgcagtgagctaagattgcaccactgcactccaacctgggcgacaagagtgaaactgtgtctctcaaaaaaaaaaaaaaacaaacaaaaacaaaaacaaaacaaaacaaaacaaaacaaaacaggtaaggattcccctgttttcctctctttaattttaaagttatcagttccgtaaagtctctgtaaccaaacatactgaagacagcaacagaagtcacgttcagggactggctcacacctgtaatcccagcactttgggagatggaggtaaaaggatctcttgagcccaggagttcaagaccagcttgggcaacatagcaagactccatctcttaaaaaataaaaatagtaacattagccaggtgtagcagcacacatctgcagcagctactcaggaggctgaggtggaaagatcgcttgtgcacagaagttcgaggctgcagtgagctatatgatcatgtcactgcactccagcctgtgtgaccgagcaagaccctatctcaaaaaaattaattaattaattaattaattaatttaaaaaggaagtcatgttcatttactttccacttcagtgtgtatcgtgtagtattttggaggttggaaagtgaaacgtaggaatcctgaagattttttccacttctagtttgcagtgctcagtgcacaatatacattttgctgaatgaataaacagaaatagggaagtaaacctacaaatattttagggagaagctcacttcttccttttctcaggaaaccaagcaagcaaacatatcgttccaattttaaaacccagtgaccaaagcctttggaactatgaatttgcaactgtcataggtttatggatattgctgtggagaagctcaattttcagtgtttgaactgaaccctttcttgttagggaacgtgtgaaagaagaattgtggggaaaaaaaagcaagcataaccaaagatcatcagcagtgaagaatctaggctgtggctgagagaaccagaggcctctaaaatggacccgagtcgatcttcagaacagggatctaccatgcaggagcttcttgtgctcacacaaatctgtaaatgggaacattgtacattgtcgaatttaaatgatattaattttctcaagctatttttgttactattttcctaaaattgaatatttgcagggagcacttatactttttcctaatgtctgtataacaaatttctatgcaagtacatgaataaattatgctcacagctca

SEQ ID NO: 135 - DNA, 401 bp, Homo sapiens
ggaaggatgtcccaagccggcaagaaccctgtggtctggcgggtgtccagcgggtggagccagagagctcccagccatgggccacctagctgaaggctgataagtactatctgcacatccaagcaggcaggagacccagataaaagacctatgacatctgcatggctggctgtgggattacatatatatattatatatatwtttaaaagtatatgtatatatgtatatatattacatatatacacacatatatatctacacatatgtaatatatgtatatatgtatatttattatatgtatatatctacatatatacatgtatgtatgtaatatagatgtatatatgttacatatatacacacatatacatatatacacatatatttttttaaaattctgc

SEQ ID NO: 136 - DNA, 401 bp, Homo sapiens
gtcccaagccggcaagaaccctgtggtctggcgggtgtccagcgggtggagccagagagctcccagccatgggccacctagctgaaggctgataagtactatctgcacatccaagcaggcaggagacccagataaaagacctatgacatctgcatggctggctgtgggattacatatatatattatatatatatttaaaartatatgtatatatgtatatatattacatatatacacacatatatatctacacatatgtaatatatgtatatatgtatatttattatatgtatatatctacatatatacatgtatgtatgtaatatagatgtatatatgttacatatatacacacatatacatatatacacatatatttttttaaaattctgcactcataa

SEQ ID NO: 137 - DNA, 401 bp, Homo sapiens
agatattttaattcagttttagatttccctgctatagaatgtttgcttcctgacttttaaagaggcagcagagtatgtccaagaattctcacctgggaagacagacattccagggaaacactgatatttttgttttctcctcactgggaaaaaggcagctggccttcagaaaacaacttgcatgtaccttatcttccccamcctcccaacagcccctcaggcccttgctggagtcttacatctgtgtagaagttgtgccaagtctaagaaaatttcaccccaggagcagtcgcttagcttccgagtccacacagcagcaacctgccccatggtttttagtttgacaccgtgttgatccttttcagttgtaaatctttccaaaaatatgagtaggggccaga

SEQ ID NO: 138 - DNA, 401 bp, Homo sapiens
aactgctgcttcacttgtttgttgacttgaaccgaaccttgggtggcattaatgtgcctggcccaagactgaaaaattaagaaccaccagagctgacctattccataagacccagtctgcctgccacgtactgagtgaatctggatgatgcccactctgatccttggttttctcttctataaaatgaaggcttgaactacrtggtctctaaaatcctacctagctctcaaatttctcttggttctaggaaaatattgatgttgagctcaaggaaggggttctccaaggtgtgtgattttggtggtagaggaaaggccggtgccaggcaggggcagaaggagacgctgtctacactgagaaaatgtgacaacccctgcttgtctcttttttcattcttcatt

SEQ ID NO: 139 - DNA, 401 bp, Homo sapiens
ctaaaaaaaaataaaataaattaaaataaacacagctggatgtggtggcacaggaaaaaaaaataccatttaggagtctcttaaaggcagcttgtgaatgcttacaaagcgtggctagtatcttattacagaaaacagagcccacatcatgcatccttcttctcacatttcataaacaaggccaagggaaactgctgtggrgcaacctgttgctttggtgttggtccccaagatgcagccctcacaatctgcccccaaacgtgtcagaacatgaaccccctcctccccctctggaagaagcaacctcagatccaacagcagagacacgcagcagaacaaaatctgggcattggtccctgtgtaggatggcttcccgttattttttttttaagcaaagtaaa

SEQ ID NO: 140 - DNA, 401 bp, Homo sapiens
ctcctccccctctggaagaagcaacctcagatccaacagcagagacacgcagcagaacaaaatctgggcattggtccctgtgtaggatggcttcccgttattttttttttaagcaaagtaaatgaacatcaaatttccatagtcagctgctgtctttctgcccactgagagctctttggtgaaggcaaagtcctccttctycattagcggtctcccatgtggggccacatcttccctcaccaggaacccagtgggcgcgctccagcccccctcagcttgccttttgcgtggtcattagagctagggcacacgtcatgctgattcacatatttttgccctttgtcatgtattgagaaaaagtaaggatgaatggacggtctttgattggcggcgctggtgac

SEQ ID NO: 141 - DNA, 401 bp, Homo sapiens
ccccctcagcttgccttttgcgtggtcattagagctagggcacacgtcatgctgattcacatatttttgccctttgtcatgtattgagaaaaagtaaggatgaatggacggtctttgattggcggcgctggtgacgcccgtcatggtcctgtttggaaggacccttttggaactaaagctggtgacgcagcgcgcagaggmatcgcccggctaagcttggccctggcagatgggtcgcaggaacaggtatgcttccttcgtgcagcctctggctcggggaacctgggagcctgctccaaactctggtgtatcttttccgggcagagcctgggaagtgggggttggctgtgagctaagccaaaggcacagggatcttggtccaaaaagccccatggcgctca

SEQ ID NO: 142 - DNA, 401 bp, Homo sapiens
tcatgagaagacccctctctgcaggacatcctagccctacaacccatcccaattatgttgaaattagattcacaaatggcaataagtcttctatatgttgggctgtcgatttggagaaaactagtttaatctttacttaactttgggtggctcaacaggagactcgggccgctcaggctctcaatcacgtctggccagttytattatcaggtttcgaatctgtatctccaaaatctctgaggtgatgggatatttcaagccctctaaaataaataaatatatgctgggaattttgagaacatgaatttgtttattctgaaatggtccatgttcctgctttgggagttgatggaaaatgccacttgagtgttttcatttgatgctgccaccttagggtttta

SEQ ID NO: 143 - DNA, 401 bp, Homo sapiens
acccctctctgcaggacatcctagccctacaacccatcccaattatgttgaaattagattcacaaatggcaataagtcttctatatgttgggctgtcgatttggagaaaactagtttaatctttacttaactttgggtggctcaacaggagactcgggccgctcaggctctcaatcacgtctggccagttctattatcagktttcgaatctgtatctccaaaatctctgaggtgatgggatatttcaagccctctaaaataaataaatatatgctgggaattttgagaacatgaatttgtttattctgaaatggtccatgttcctgctttgggagttgatggaaaatgccacttgagtgttttcatttgatgctgccaccttagggttttatagattcagt

SEQ ID NO: 144 - DNA, 401 bp, Homo sapiens
aatctttacttaactttgggtggctcaacaggagactcgggccgctcaggctctcaatcacgtctggccagttctattatcaggtttcgaatctgtatctccaaaatctctgaggtgatgggatatttcaagccctctaaaataaataaatatatgctgggaattttgagaacatgaatttgtttattctgaaatggtccrtgttcctgctttgggagttgatggaaaatgccacttgagtgttttcatttgatgctgccaccttagggttttatagattcagttccagaaactcaaggcatttatctctttgggctgcttgtccttgcctgagctgaagcctgatgcctcccataagttggtatggctttgaaaatgggtcactacagcagaggcatggg

SEQ ID NO: 145 - DNA, 401 bp, Homo sapiens
taccatagctaacaaatagcaatgaacagatactaagttatacctattaaaaaaagttgaactctagctttggcttctattaatcccagtaaagaaatcaatatgggtaactgatttattggtagcaattacatttcaattgtattacctcactgcccaactaaccattctgctttttaaacaatgccaaactattaatcwttacacaaaaaagcagctttttatacaatgcattccagaccaaggtcattttatctcggtaaaacaatgttagataccaacatctgtgtgacttgcaatgaatcttggaggtcaggaaccaaaattaagtccatatggagctgagttggctccatagttaaagataaaaaaccgtatgaataattaagcacttatgaaaa

SEQ ID NO: 146 - DNA, 401 bp, Homo sapiens
tggttcctgacctccaagattcattgcaagtcacacagatgttggtatctaacattgttttaccgagataaaatgaccttggtctggaatgcattgtataaaaagctgcttttttgtgtaaagattaatagtttggcattgtttaaaaagcagaatggttagttgggcagtgaggtaatacaattgaaatgtaattgctascaataaatcagttacccatattgatttctttactgggattaatagaagccaaagctagagttcaactttttttaataggtataacttagtatctgttcattgctatttgttagctatggtaaatggaacaatgatggggccagaaatatccatgaggaccatttgatcacagcctggcaacacagagaagacaggctggt

SEQ ID NO: 147 - DNA, 401 bp, Homo sapiens
gataaaatgaccttggtctggaatgcattgtataaaaagctgcttttttgtgtaaagattaatagtttggcattgtttaaaaagcagaatggttagttgggcagtgaggtaatacaattgaaatgtaattgctaccaataaatcagttacccatattgatttctttactgggattaatagaagccaaagctagagttcaaytttttttaataggtataacttagtatctgttcattgctatttgttagctatggtaaatggaacaatgatggggccagaaatatccatgaggaccatttgatcacagcctggcaacacagagaagacaggctggtttctctatgtgggctttcagtgtttctttggtagtgtcttatgtggctgtggcttcaacattccac

SEQ ID NO: 148 - DNA, 401 bp, Homo sapiens
aagccaaagctagagttcaactttttttaataggtataacttagtatctgttcattgctatttgttagctatggtaaatggaacaatgatggggccagaaatatccatgaggaccatttgatcacagcctggcaacacagagaagacaggctggtttctctatgtgggctttcagtgtttctttggtagtgtcttatgtgkctgtggcttcaacattccacaattatgccttccagggtctgatgattttggcgtttccctgcttcccaattgacctggctgtgctgttggctgttcttgcacactcaaggtggttttgccattggcttcctccctcagcctgcctctgggattatgccactgctattcttttttatctaccatcagcacaatgaaatcat

SEQ ID NO: 149 - DNA, 401 bp, Homo sapiens
caattatgccttccagggtctgatgattttggcgtttccctgcttcccaattgacctggctgtgctgttggctgttcttgcacactcaaggtggttttgccattggcttcctccctcagcctgcctctgggattatgccactgctattcttttttatctaccatcagcacaatgaaatcatcatttttgtcttcaaggtascaaattctggtgatattggtgctttcttgcagctacttatcatgagaagtgaatggtctcatagtgaacacagtcatggttatagtgttcatacgttccagagacatgtttcctataattatgccctgcacatttttctatcatacaatccttagattacagctctttggttttcaacagctttgtccaattccatcttt

SEQ ID NO: 150 - DNA, 401 bp, Homo sapiens
atccttagattacagctctttggttttcaacagctttgtccaattccatctttcccagtttctctaccttgatgaaatatccttcttgcctggttttacatatttaaataacaaattccaaaagtaaagagtatctgaggcagtcacatgacataaggacaaattcaagccatcttggacttgcagagggtggggagaccrtgtcaacacacacaattttaaaaatttcttccctttcaatcttttaaaaacaaaactttttataaaataaaaatgtaatttaaaaaggctacctgtcttggcaagtagctgatcagcctgcattggtgagcaggccattccataacctggtttcttgctccttaattgacagcatggagctaacgtacttaatttcagct

SEQ ID NO: 151 - DNA, 401 bp, Homo sapiens
aataacaaattccaaaagtaaagagtatctgaggcagtcacatgacataaggacaaattcaagccatcttggacttgcagagggtggggagaccgtgtcaacacacacaattttaaaaatttcttccctttcaatcttttaaaaacaaaactttttataaaataaaaatgtaatttaaaaaggctacctgtcttggcaagyagctgatcagcctgcattggtgagcaggccattccataacctggtttcttgctccttaattgacagcatggagctaacgtacttaatttcagctctttctacgtgatttgactcattctgttaacattaactgtttttcagtcttctcaactagactgaactccttaagtgcaagaaatacacgcttagtaaatgtttgt

SEQ ID NO: 152 - DNA, 401 bp, Homo sapiens
acacacaattttaaaaatttcttccctttcaatcttttaaaaacaaaactttttataaaataaaaatgtaatttaaaaaggctacctgtcttggcaagtagctgatcagcctgcattggtgagcaggccattccataacctggtttcttgctccttaattgacagcatggagctaacgtacttaatttcagctctttctaygtgatttgactcattctgttaacattaactgtttttcagtcttctcaactagactgaactccttaagtgcaagaaatacacgcttagtaaatgtttgttggaccagacactgcaccttatgaaattaaagaccagaacattctcatggtagcattacagacactgatggcaaaggtactgtgggatttgggtttggctaa

SEQ ID NO: 153 - DNA, 401 bp, Homo sapiens
atacacgcttagtaaatgtttgttggaccagacactgcaccttatgaaattaaagaccagaacattctcatggtagcattacagacactgatggcaaaggtactgtgggatttgggtttggctaataagctctgtggtggtgtttcagaaggaaaatggtgctctcttagttctatggaacatagtggtccagatcttctrctgtaaccaggcccaaagctggctaatctggagggctctgccttagggatacttataagctctgtccttccctcaaggagccagaggaagagatagccatggaggacagccccactatggttagagtggacagccccactatggttaggggtgaaaaccaggtttcgccatgtcaagggagaaggtgcttccccaaagct

SEQ ID NO: 154 - DNA, 401 bp, Homo sapiens
tgacgaacaccacttgggatatgccccggagaatccagtcaatgaactggagcaccacgggtttgtctggaagggatgaggtaaagcagagctaaggaagctgccaagtgatactcttcaaccacaggcccaccctgagcaaaatcagcaaaacctcctggaagtgaaacatttttgatatttctttaatatttctctctsaacccattgttgcattagtggcacccagatccctgaaagcctctatctactgtatgctcaacccacactgcccctcctagaatctaagctcctgaaagcatctaatgcacttattttcatgcttataggtacaaattctgaagccaaaggaataaatttaagttttaagtactgaaactacaggatgaagccaggtgcct

SEQ ID NO: 155 - DNA, 401 bp, Homo sapiens
ctggcttcatcctgtagtttcagtacttaaaacttaaatttattcctttggcttcagaatttgtacctataagcatgaaaataagtgcattagatgctttcaggagcttagattctaggaggggcagtgtgggttgagcatacagtagatagaggctttcagggatctgggtgccactaatgcaacaatgggttgagagaraaatattaaagaaatatcaaaaatgtttcacttccaggaggttttgctgattttgctcagggtgggcctgtggttgaagagtatcacttggcagcttccttagctctgctttacctcatcccttccagacaaacccgtggtgctccagttcattgactggattctccggggcatatcccaagtggtgttcgtcaacaacc

SEQ ID NO: 156 - DNA, 401 bp, Homo sapiens
cctttcaagccttctcagctcccttctgagacacaggggctgaccagttactgtgggcaacagtgataaaaccacatccttcccaggataaacaacatttagtccacagaactgtttatatttgtttttagtcagaggtcagggaatcagttacagtctcttgctcttgatatctgaataaatggctggtctaaatgatgscagattcttgtggcattacgtgctaaccagaactaagctacaagtatttccctggagaggttctgaagggatcttctttaatgattgataaaattatttgtcgtcagcattctatttgggaaaaagtgcatatgaattcagaaaaagttttagtggcttaataacccccgttatatcttgttgctatgatgagtttagga

SEQ ID NO: 157 - DNA, 401 bp, Homo sapiens
tcccttctgagacacaggggctgaccagttactgtgggcaacagtgataaaaccacatccttcccaggataaacaacatttagtccacagaactgtttatatttgtttttagtcagaggtcagggaatcagttacagtctcttgctcttgatatctgaataaatggctggtctaaatgatgccagattcttgtggcattaygtgctaaccagaactaagctacaagtatttccctggagaggttctgaagggatcttctttaatgattgataaaattatttgtcgtcagcattctatttgggaaaaagtgcatatgaattcagaaaaagttttagtggcttaataacccccgttatatcttgttgctatgatgagtttaggaaactcattcttcatagaca

SEQ ID NO: 158 - DNA, 401 bp, Homo sapiens
tggtctaaatgatgccagattcttgtggcattacgtgctaaccagaactaagctacaagtatttccctggagaggttctgaagggatcttctttaatgattgataaaattatttgtcgtcagcattctatttgggaaaaagtgcatatgaattcagaaaaagttttagtggcttaataacccccgttatatcttgttgctrtgatgagtttaggaaactcattcttcatagacagtgcaaaggtcagctcagctcctggagaaaagaataaccatgaattccaattgagtggattctgacttaagaagccttagtgagtcttctgatatattgattagattaaaaatagcacacactttataaattgatctgtcattgaagaagtgatgagctgactctca

SEQ ID NO: 159 - DNA, 401 bp, Homo sapiens
ttgggaaaaagtgcatatgaattcagaaaaagttttagtggcttaataacccccgttatatcttgttgctatgatgagtttaggaaactcattcttcatagacagtgcaaaggtcagctcagctcctggagaaaagaataaccatgaattccaattgagtggattctgacttaagaagccttagtgagtcttctgatataytgattagattaaaaatagcacacactttataaattgatctgtcattgaagaagtgatgagctgactctcaccagggcagtagatagctccccactagccagttcctttagggagggaaccagtattccaggtgtctgagatcaacgcataatcccaatccccagtgtggtcattacacaactaagctcttgtaacactgg

SEQ ID NO: 160 - DNA, 401 bp, Homo sapiens
gtgttagtttcaaatgttttactttccttggtctgaaaagactgcattaaaatggaaattctctgttttaagtaaatatatgtcttcctgtggctttaactatggcattccacaatttgtagatgttgccattaattttccactgatcaaactcaagcattaacatctccaagtcagttgttgagaggacaagtctgcatrgctctctactgtcatgtgtagtcccagtctctgagttgtacctttgcaaattgtatcacctcccatttgccctcaaggattatttaagggaaacaaagaacttttgaatagggaaccccacatttaatgttcatctggattaatgtacgtgacatcatcttgcctgttgcaatggtgcctcctggcccagttagaaacaa

SEQ ID NO: 161 - DNA, 401 bp, Homo sapiens
gctttaactatggcattccacaatttgtagatgttgccattaattttccactgatcaaactcaagcattaacatctccaagtcagttgttgagaggacaagtctgcatggctctctactgtcatgtgtagtcccagtctctgagttgtacctttgcaaattgtatcacctcccatttgccctcaaggattatttaagggaracaaagaacttttgaatagggaaccccacatttaatgttcatctggattaatgtacgtgacatcatcttgcctgttgcaatggtgcctcctggcccagttagaaacaagccaagaagcagctgtcacactatcccttaccagcccctgcagtgtggctcactggctatagcacctcctgctcgagcccagcattaggcct

SEQ ID NO: 162 - DNA, 401 bp, Homo sapiens
ctcaagcattaacatctccaagtcagttgttgagaggacaagtctgcatggctctctactgtcatgtgtagtcccagtctctgagttgtacctttgcaaattgtatcacctcccatttgccctcaaggattatttaagggaaacaaagaacttttgaatagggaaccccacatttaatgttcatctggattaatgtacgtracatcatcttgcctgttgcaatggtgcctcctggcccagttagaaacaagccaagaagcagctgtcacactatcccttaccagcccctgcagtgtggctcactggctatagcacctcctgctcgagcccagcattaggcctcacctactcacttcaccatctttactcccccatccccctacagacatcatccttgagtg

SEQ ID NO: 163 - DNA, 401 bp, Homo sapiens
tatcccttaccagcccctgcagtgtggctcactggctatagcacctcctgctcgagcccagcattaggcctcacctactcacttcaccatctttactcccccatccccctacagacatcatccttgagtgacaggcccttgggaagtggatcctgtgcctttcacggtgccagacgttgccaactctcagagctgtgggartcctgccttgtcaggtcaatcaatctaggtgcccatcaatggtggattatataaagaatatgtggtgcatatacaacacgaactactacatagccataaaaaggattgaaatcaagtcctttgcagcagcatggatgtatctggagaccaatatcctaagtgaattaatgtagtaacagaaaatcaaataccacacgttt

SEQ ID NO: 164 - DNA, 401 bp, Homo sapiens
tggctatagcacctcctgctcgagcccagcattaggcctcacctactcacttcaccatctttactcccccatccccctacagacatcatccttgagtgacaggcccttgggaagtggatcctgtgcctttcacggtgccagacgttgccaactctcagagctgtgggaatcctgccttgtcaggtcaatcaatctaggtgyccatcaatggtggattatataaagaatatgtggtgcatatacaacacgaactactacatagccataaaaaggattgaaatcaagtcctttgcagcagcatggatgtatctggagaccaatatcctaagtgaattaatgtagtaacagaaaatcaaataccacacgttttcacttacaattaggagctaaacactgggtaa

SEQ ID NO: 165 - DNA, 401 bp, Homo sapiens
aatacatctatgtaacaaacctgcacatgtaccccctcaatctaaagaaggagaagaagacggggaagaaatgagattgaatactaagcaaaaagtaacctcagaaagaactgggtgctcaacatgcacataattaaatgggatacttctccaagtaagagaaaagcaattgttcttctttgcaataactttgaaatgtgygtttggagacaacaaaatagaagcatcaggacacaaaaatgtatactaacctggaagattaatgttgataagatcaaagacactgtgaaagtgaatttacatttcaggaatcttatatctctcaccaagaaatcaaacttaagcaacagtttcatatgctaaaagcgctcttcaagtcagaggctcttgatttaaaagaa

SEQ ID NO: 166 - DNA, 401 bp, Homo sapiens
gccaccctggtgggagtactcatggctgtcttttcggacaagggagactatttctggtggctgttactccctgtatgtgctatgtccatgacttggtaagttacaattggttttcaaaatgcctttttgaaaaaaaaaacatggcagaaggagggaatgggagttgttatatggcagagtttcagttttgcaagatgaaawatgttctctgaatgtatagtggtgatggttgtacaacaatgtgattgtccttaatgtcattgagctgcacacttaaaaatggttagccgggtgcggtggttcttgtttgtagtccaaactattcagaaggctgagggggaaggatcacttgagcccaggagttaggggctgcagtgagctatgattgcgtcaccgcactc

SEQ ID NO: 167 - DNA, 401 bp, Homo sapiens
gtatgtgctatgtccatgacttggtaagttacaattggttttcaaaatgcctttttgaaaaaaaaaacatggcagaaggagggaatgggagttgttatatggcagagtttcagttttgcaagatgaaatatgttctctgaatgtatagtggtgatggttgtacaacaatgtgattgtccttaatgtcattgagctgcacayttaaaaatggttagccgggtgcggtggttcttgtttgtagtccaaactattcagaaggctgagggggaaggatcacttgagcccaggagttaggggctgcagtgagctatgattgcgtcaccgcactccagttctccgaacctccttgcttgggctaagtgaggaggaggaggaggaggagaaggatggaaaggaggagg

SEQ ID NO: 168 - DNA, 401 bp, Homo sapiens
attggattccagtagattctgtctattggaaacagaaacaaccattttaaaagatgtatatttccttacaaccagttatttggccttttgtctgatctggctacacatccactaatacctctcaaccagaggtggctgcacattgacacttccatggggaagggaaacagtgctgcaatgaagatacgagtgcaggtgtcyttttggtagaaacacactgatgcacgtggcccccacatacacttgactcctccctcccaagactctactgtcattggtctgcggtagcgcctgggctttgggagtttctaaagcttcccagatgactctaaagtatagccaaagttgagacccacttcctccatcattgcctctcaaacttgagcaatatgagaatcacc

SEQ ID NO: 169 - DNA, 401 bp, Homo sapiens
ggtctcccttagaaaaaatttttttgctgaattccttttttttcaaacccaaatccttcaaactagtttttatgttgacaatgtcttacatcctttttctggaaacaaagatttccttctttctatattgtagttaaatataaaatactaatatgcacataaataagcacagcctgctgtgggcagtgtctgcagaagggmtgcccacccttactgtacccacgggtgtgtggacgaggacctacctgtagagctaaactcttcaggaagtaatttgggccctgctctgaagaataggttcgtgggaaggaggcctagcctgtaagtgctcaccacgctcccttccacaatccaggaaaatgggagttctggtctttaagtgatggctctttgattgggcc

SEQ ID NO: 170 - DNA, 401 bp, Homo sapiens
tgaaggcagaaaggccaccatagtcctgagcatctaggagactctgacacccgtggacagttgaccaggaggccattgcgtatcttgcagagccagggcgaatgtgagaagccagtgtctcttacctccagggcactgaggtcagaccaggagatatttggagctgtagttataggtatgaccagtttggctggaaagaayggattgtaatgtcctgtggctgaaaggtacattgacaacgccatgttgaaagggagggtgaagacggggaggtcccatttgctgagcatggaattcaatgcacttgagaaaattgggctaaaaaaagaacagagcaaaagaatgtgcatgtgacaaagcaggctgacacacggaatcgctatactctggaggggctttgt

SEQ ID NO: 171 - DNA, 401 bp, Homo sapiens
tccagagcattagggtctaagggattttttaaaattactatttagtcaagctgatttttctgccttttcccctaaacatctacagtgctaaccccagagtacagttccactgggagtcactctatcgtaagcttgggggtgggggtgatgggagccagcccttaaggcatgtggcctccagcctggttttaaatcttccayagtctactccctccaatcaaaaaactggatgcttactcttagagcttctgacagaacctctctattctgcttttccttatggcatagctcatagaacatctacaataatttagggttcccaagctttggtaggcatcagaatcacctggggagctttaaatacccaaacaggcttcatctcagaccctctaaatcacaat

SEQ ID NO: 172 - DNA, 401 bp, Homo sapiens
catagctcatagaacatctacaataatttagggttcccaagctttggtaggcatcagaatcacctggggagctttaaatacccaaacaggcttcatctcagaccctctaaatcacaatctctaagggtggggcctggaacctgttttaacaaactccccaaattgtgatgcgggccagagtttgagaaccactgtatcaargggtgaatcctatgtatctctttaaagatggctataaagagattctgtattttttaaaacctggttaacccaaatcaaattccagctcttcctgttggtgtgtaataaatatgtttaaggtttctggattatcaagaacaagagaacacctgaaattagaagaaaaccaaagaaaccttacctttttaatgtgctctccc

SEQ ID NO: 173 - DNA, 401 bp, Homo sapiens
ttcccaagctttggtaggcatcagaatcacctggggagctttaaatacccaaacaggcttcatctcagaccctctaaatcacaatctctaagggtggggcctggaacctgttttaacaaactccccaaattgtgatgcgggccagagtttgagaaccactgtatcaaggggtgaatcctatgtatctctttaaagatggcyataaagagattctgtattttttaaaacctggttaacccaaatcaaattccagctcttcctgttggtgtgtaataaatatgtttaaggtttctggattatcaagaacaagagaacacctgaaattagaagaaaaccaaagaaaccttacctttttaatgtgctctcccactgtcaggttatgaaacgcccttttgtcttct

SEQ ID NO: 174 - DNA, 401 bp, Homo sapiens
attccagctcttcctgttggtgtgtaataaatatgtttaaggtttctggattatcaagaacaagagaacacctgaaattagaagaaaaccaaagaaaccttacctttttaatgtgctctcccactgtcaggttatgaaacgcccttttgtcttctttgttgagtgatcaaaacacacgaggagctcaagtcaccttctccytagcttcttgccagaaaactaaagggagcacctggaaataattcagaaggaaaaaatcaaagattcattagaactacccatgaaaaataacagtataaaatagcattaatcgatctagaactgcactaacacaggagcctctagccccatgtggctatataaatttagatgtagattagttaaaaattgagttcctcaac

SEQ ID NO: 175 - DNA, 401 bp, Homo sapiens
gtgtgtaataaatatgtttaaggtttctggattatcaagaacaagagaacacctgaaattagaagaaaaccaaagaaaccttacctttttaatgtgctctcccactgtcaggttatgaaacgcccttttgtcttctttgttgagtgatcaaaacacacgaggagctcaagtcaccttctccctagcttcttgccagaaaaytaaagggagcacctggaaataattcagaaggaaaaaatcaaagattcattagaactacccatgaaaaataacagtataaaatagcattaatcgatctagaactgcactaacacaggagcctctagccccatgtggctatataaatttagatgtagattagttaaaaattgagttcctcaacctctctagccacatctcag

SEQ ID NO: 176 - DNA, 401 bp, Homo sapiens
cctctagccccatgtggctatataaatttagatgtagattagttaaaaattgagttcctcaacctctctagccacatctcaggtgcttgatagccacacgtggctaggacccactgtattagacagcacagatacagactattccatcatctcggaaagttatcctgcacagtgctgatctggggcaggggaagccttgtscttctcactctgaatgaacagcccatcctcagcaccaaccccaaccctatggctacctgagagagagttctgcagccaagtccaaaaacaaacaaacaaacaaaaaaagcatatgccatctttgccaagttccctggtctagaaatagcaaaatgtctagacatgaagactcagcatgggctggaagaatttagagtcca

SEQ ID NO: 177 - DNA, 401 bp, Homo sapiens
cactgtattagacagcacagatacagactattccatcatctcggaaagttatcctgcacagtgctgatctggggcaggggaagccttgtccttctcactctgaatgaacagcccatcctcagcaccaaccccaaccctatggctacctgagagagagttctgcagccaagtccaaaaacaaacaaacaaacaaaaaaagcrtatgccatctttgccaagttccctggtctagaaatagcaaaatgtctagacatgaagactcagcatgggctggaagaatttagagtccatcttagggtagagtcaaactcacactatggtctggtgcccttagccaatgttagactcagcctaatataagaggggagaagacacttccccttgtgccaaagctggggctc

SEQ ID NO: 178 - DNA, 401 bp, Homo sapiens
tctttgccaagttccctggtctagaaatagcaaaatgtctagacatgaagactcagcatgggctggaagaatttagagtccatcttagggtagagtcaaactcacactatggtctggtgcccttagccaatgttagactcagcctaatataagaggggagaagacacttccccttgtgccaaagctggggctccctctggyagagtcactgcctccagaaggtctttggtacatacacgacctagcaatggtggagagggcaagatgggaactgaggaaaacatctttcagtaaatggccttgctcaaaagggacatgctatggctaattatgcctatcctagccctaccagaagttcagctgtaaagaatgatcacttgttaggttcagttaaaccttgt

SEQ ID NO: 179 - DNA, 401 bp, Homo sapiens
atttagagtccatcttagggtagagtcaaactcacactatggtctggtgcccttagccaatgttagactcagcctaatataagaggggagaagacacttccccttgtgccaaagctggggctccctctggtagagtcactgcctccagaaggtctttggtacatacacgacctagcaatggtggagagggcaagatgggarctgaggaaaacatctttcagtaaatggccttgctcaaaagggacatgctatggctaattatgcctatcctagccctaccagaagttcagctgtaaagaatgatcacttgttaggttcagttaaaccttgttcactcctgagaactgcaattctgtgaacagaataactaaattcaggcctcagccagaaagtagaattat

SEQ ID NO: 180 - DNA, 401 bp, Homo sapiens
aagtagaattatgacatttccatgtatttttgtgttttgagacctgcttgacagttgttcataactagaataagctaaaaatatctttgtttaaatgaatacatgttccacttaatgacagaaaagtaaattcacaaacttgctaaaaattacttctaaattgtggacaagataacctggctttgggtctctggctttagygtaagcatccaaattgcatagtgataataatctctattgaacatagggatgcatggatagattaaatcaccctcaacactgatggacatttgaaagcaaaagaagtgtcagctgtggtccttgccatccccagtaggaggcaaggcagatcctcatagccaggagcagtgagtggcaccaagctgggagcttaacagtga

SEQ ID NO: 181 - DNA, 401 bp, Homo sapiens
acagttgttcataactagaataagctaaaaatatctttgtttaaatgaatacatgttccacttaatgacagaaaagtaaattcacaaacttgctaaaaattacttctaaattgtggacaagataacctggctttgggtctctggctttagtgtaagcatccaaattgcatagtgataataatctctattgaacatagggawgcatggatagattaaatcaccctcaacactgatggacatttgaaagcaaaagaagtgtcagctgtggtccttgccatccccagtaggaggcaaggcagatcctcatagccaggagcagtgagtggcaccaagctgggagcttaacagtgaccaaggccaagtgtcagtgcaagcaggagagcacagggggagctttgaga

SEQ ID NO: 182 - DNA, 401 bp, Homo sapiens
atgttccacttaatgacagaaaagtaaattcacaaacttgctaaaaattacttctaaattgtggacaagataacctggctttgggtctctggctttagtgtaagcatccaaattgcatagtgataataatctctattgaacatagggatgcatggatagattaaatcaccctcaacactgatggacatttgaaagcaaaaraagtgtcagctgtggtccttgccatccccagtaggaggcaaggcagatcctcatagccaggagcagtgagtggcaccaagctgggagcttaacagtgaccaaggccaagtgtcagtgcaagcaggagagcacagggggagctttgagaaggcatgtgttgcatgcaccagggaagggctggtgtatctctggggataaag

SEQ ID NO: 183 - DNA, 401 bp, Homo sapiens
agtgcaagcaggagagcacagggggagctttgagaaggcatgtgttgcatgcaccagggaagggctggtgtatctctggggataaagctgaaggatgactgggatttttctgtaatcaaagagagagaattttaaatggtattaacactgttcttgaaagaggtaaggtatgtccaatctaaaattacattgtaggagttygtgggtgtcctgtgggtttctgttcagttgttttggtagcctcatttttcttaaatttcttttgcagttgttgaaatctataccagtgggagttggtcagatctatggctgtgataatccatggacagggggcattttcctgggagccatcctactctcctccccactcatgtgcctgcatgctgccataggatcattgc

SEQ ID NO: 184 - DNA, 401 bp, Homo sapiens
ggtgaaaccctgtatctacaaaaaatacaaaaattagccaggcatggtggtgggtgcctgtagttccagctacttgggaggctgaggtgggaaaatgacttgagcccaggaggaggaggctgcagtgagctaagattgcaccactgcactccaacctgggcgacaagagtgaaactgtgtctctcaaaaaaaaaaaaaaamaaacaaaaacaaaaacaaaacaaaacaaaacaaaacaaaacaggtaaggattcccctgttttcctctctttaattttaaagttatcagttccgtaaagtctctgtaaccaaacatactgaagacagcaacagaagtcacgttcagggactggctcacacctgtaatcccagcactttgggagatggaggtaaaaggatct

SEQ ID NO: 185 - DNA, 401 bp, Homo sapiens
agagtgtgtgtgttctagaagcctcagttctctcgttctagtcccagaggtcaccagagtttcctgcaaatccactttaggttctatgagcttccttaccttttctacttaagctagtatagaaagtataactgacactttcaactaagagtctcaactaattaaccaatccttaacatttgagttaaactttcttaaacycatggttctttaaaatactttctcattctgtttatatatcctcccaacagaataagagagagagagagagagagagagagagagagagtgtgtgtgtgtgtgtgtgtgtgtgtggtttaaaacaatagaaatttattctctcacagttctggaggccagaaatccaaaactaaggtattgccagagttgtgtttttccct

SEQ ID NO: 186 - DNA, 401 bp, Homo sapiens
gcttccttaccttttctacttaagctagtatagaaagtataactgacactttcaactaagagtctcaactaattaaccaatccttaacatttgagttaaactttcttaaacccatggttctttaaaatactttctcattctgtttatatatcctcccaacagaataagagagagagagagagagagagagagagagagagwgtgtgtgtgtgtgtgtgtgtgtgtggtttaaaacaatagaaatttattctctcacagttctggaggccagaaatccaaaactaaggtattgccagagttgtgtttttccctgagaaagaagattccatgcctgtcaactggctcctggtggtagtcagcaatccttgatgttccctggcatttcctggcttgtagtggca

SEQ ID NO: 187 - DNA, 401 bp, Homo sapiens
ttccttaccttttctacttaagctagtatagaaagtataactgacactttcaactaagagtctcaactaattaaccaatccttaacatttgagttaaactttcttaaacccatggttctttaaaatactttctcattctgtttatatatcctcccaacagaataagagagagagagagagagagagagagagagagagtgwgtgtgtgtgtgtgtgtgtgtgtggtttaaaacaatagaaatttattctctcacagttctggaggccagaaatccaaaactaaggtattgccagagttgtgtttttccctgagaaagaagattccatgcctgtcaactggctcctggtggtagtcagcaatccttgatgttccctggcatttcctggcttgtagtggcatc

SEQ ID NO: 188 - DNA, 401 bp, Homo sapiens
cagaataagagagagagagagagagagagagagagagagagtgtgtgtgtgtgtgtgtgtgtgtgtggtttaaaacaatagaaatttattctctcacagttctggaggccagaaatccaaaactaaggtattgccagagttgtgtttttccctgagaaagaagattccatgcctgtcaactggctcctggtggtagtcagyaatccttgatgttccctggcatttcctggcttgtagtggcatcactccaacctttgtctatatcttcagatggttttcttctctttgcgtgtctgtggctctgtggctccatgttttcctctcttttcccttacaaagatgccagtcattggatgtagggcccaccctaatccaacgttacctcatcttaactagctaca

SEQ ID NO: 189 - DNA, 401 bp, Homo sapiens
agagagagagagagagagagagagagtgtgtgtgtgtgtgtgtgtgtgtgtggtttaaaacaatagaaatttattctctcacagttctggaggccagaaatccaaaactaaggtattgccagagttgtgtttttccctgagaaagaagattccatgcctgtcaactggctcctggtggtagtcagcaatccttgatgttcyctggcatttcctggcttgtagtggcatcactccaacctttgtctatatcttcagatggttttcttctctttgcgtgtctgtggctctgtggctccatgttttcctctcttttcccttacaaagatgccagtcattggatgtagggcccaccctaatccaacgttacctcatcttaactagctacatctgcaaaggctctg

SEQ ID NO: 190 - DNA, 401 bp, Homo sapiens
gaaagaagattccatgcctgtcaactggctcctggtggtagtcagcaatccttgatgttccctggcatttcctggcttgtagtggcatcactccaacctttgtctatatcttcagatggttttcttctctttgcgtgtctgtggctctgtggctccatgttttcctctcttttcccttacaaagatgccagtcattggatktagggcccaccctaatccaacgttacctcatcttaactagctacatctgcaaaggctctgtttccaaataaagtcacattctgaggttctggatagacattagccttagggggcacactattcaacctattacacatatataatatgtcctaccattgcccacttgagagggcttggttgttttacatgccaaaagcagt

SEQ ID NO: 191 - DNA, 401 bp, Homo sapiens
caactggctcctggtggtagtcagcaatccttgatgttccctggcatttcctggcttgtagtggcatcactccaacctttgtctatatcttcagatggttttcttctctttgcgtgtctgtggctctgtggctccatgttttcctctcttttcccttacaaagatgccagtcattggatgtagggcccaccctaatccaaygttacctcatcttaactagctacatctgcaaaggctctgtttccaaataaagtcacattctgaggttctggatagacattagccttagggggcacactattcaacctattacacatatataatatgtcctaccattgcccacttgagagggcttggttgttttacatgccaaaagcagtgagcatacctaccactaagag

SEQ ID NO: 192 - DNA, 401 bp, Homo sapiens
cctttgtctatatcttcagatggttttcttctctttgcgtgtctgtggctctgtggctccatgttttcctctcttttcccttacaaagatgccagtcattggatgtagggcccaccctaatccaacgttacctcatcttaactagctacatctgcaaaggctctgtttccaaataaagtcacattctgaggttctggatasacattagccttagggggcacactattcaacctattacacatatataatatgtcctaccattgcccacttgagagggcttggttgttttacatgccaaaagcagtgagcatacctaccactaagagcgtggcttctaaatgccattcttaaaacctgcacattctccacatgaatcccagaacttaaagtaacaacaacaa

SEQ ID NO: 193 - DNA, 401 bp, Homo sapiens
ttacacatatataatatgtcctaccattgcccacttgagagggcttggttgttttacatgccaaaagcagtgagcatacctaccactaagagcgtggcttctaaatgccattcttaaaacctgcacattctccacatgaatcccagaacttaaagtaacaacaacaacaaaaaagaaccaaatagtgatcgaagaatgatyggggaaaactgaaaaagacacagaagtcagtttgaaagaacttccactggctaaatttgggacagtttgagcatgaaaataaataataacagtaatatgtcctctcaaataatatagaaaaccatgaatcaatacttatatacctaaatacatacatacaaacaaacatacatacatgtgctaattgaaaatagagatat

SEQ ID NO: 194 - DNA, 401 bp, Homo sapiens
aaaacctgcacattctccacatgaatcccagaacttaaagtaacaacaacaacaaaaaagaaccaaatagtgatcgaagaatgatcggggaaaactgaaaaagacacagaagtcagtttgaaagaacttccactggctaaatttgggacagtttgagcatgaaaataaataataacagtaatatgtcctctcaaataatakagaaaaccatgaatcaatacttatatacctaaatacatacatacaaacaaacatacatacatgtgctaattgaaaatagagatatttgtcattcctttttcagcaaatataatttcatcctatggatatatatggtagttcatttaagtgttctattactgattcaagttaattccaatgctttactattttaatagtaa

SEQ ID NO: 195 - DNA, 401 bp, Homo sapiens
ctgcacattctccacatgaatcccagaacttaaagtaacaacaacaacaaaaaagaaccaaatagtgatcgaagaatgatcggggaaaactgaaaaagacacagaagtcagtttgaaagaacttccactggctaaatttgggacagtttgagcatgaaaataaataataacagtaatatgtcctctcaaataatatagaaraccatgaatcaatacttatatacctaaatacatacatacaaacaaacatacatacatgtgctaattgaaaatagagatatttgtcattcctttttcagcaaatataatttcatcctatggatatatatggtagttcatttaagtgttctattactgattcaagttaattccaatgctttactattttaatagtaatgcaa

SEQ ID NO: 196 - DNA, 401 bp, Homo sapiens
atcccagaacttaaagtaacaacaacaacaaaaaagaaccaaatagtgatcgaagaatgatcggggaaaactgaaaaagacacagaagtcagtttgaaagaacttccactggctaaatttgggacagtttgagcatgaaaataaataataacagtaatatgtcctctcaaataatatagaaaaccatgaatcaatacttayatacctaaatacatacatacaaacaaacatacatacatgtgctaattgaaaatagagatatttgtcattcctttttcagcaaatataatttcatcctatggatatatatggtagttcatttaagtgttctattactgattcaagttaattccaatgctttactattttaatagtaatgcaatccattttttattttataa

SEQ ID NO: 197 - DNA, 401 bp, Homo sapiens
ctcagggggtacatgtgcatgtttgttacatgagtatattttgtatttgtggggattgggcttctactgtaccctttacccaaatagtgaacattgtactcaatgggtaatttttcaaccctcgccctcctcccaacctccccccttcaggagtccccactgtctattatttctatctttgtgttcatgtttacccattgyttatctcccacttatgagtgaaaacacgtggtatttgatttcctgagttagttcactaaggatactgccctccaattccatccatattgctgcaaaggacatgatttcatttgttatggctgccgaataaattctattgagctttattttgtatttcaaaatacagacaatttaaccaattagaattaagtatgactgca

SEQ ID NO: 198 - DNA, 401 bp, Homo sapiens
aattaagtatgactgcaactagcatagactcaaagtaatagtgggtaatttctttctcctatttggaaaatctggaggcaggctgtctgtggctggtgccccaccaagtagttgaggacccaggctttttccctctttcccaagaggacctcccagtctaagatggttctggatcagtggctcttcatcagggataatttyccccctaaaggacagttgacaatgtctggagacatttttattgtcataatggggacgaggtgcaatggcatcaaaaaagtagaggccaggggtgctgctaaatgtcctacaacccctggcaacaaggaattatctaagcccaaaatgggaacggtgtggaggttgagaaatcccgttctcactccatctatcacacctgt

SEQ ID NO: 199 - DNA, 401 bp, Homo sapiens
tggtttagtcttgggagggagtatgtgtcgaggaatttatccatttcttctagattttctagtttatttgcatagaggtgtttatattattctctgatggtagtttgtatttctgtgggattggtggtgatatccccttcgtcattgtttattgcgtctatttgattcttctctcttttcttctttattagtcttgctagyggtctatcaattttgttgatcttttcaaaaaaccagctcctggattcattgattttttgaagggttttttgtgtctctattttcttcagttctgctctgatcttagttatttcttgccttctgctagcttttgaatgtgtttgctcttgcttctctagttcttttaattgtgatgttagggtgtcaattttagatatttc

SEQ ID NO: 200 - DNA, 401 bp, Homo sapiens
ggtttagtcttgggagggagtatgtgtcgaggaatttatccatttcttctagattttctagtttatttgcatagaggtgtttatattattctctgatggtagtttgtatttctgtgggattggtggtgatatccccttcgtcattgtttattgcgtctatttgattcttctctcttttcttctttattagtcttgctagtrgtctatcaattttgttgatcttttcaaaaaaccagctcctggattcattgattttttgaagggttttttgtgtctctattttcttcagttctgctctgatcttagttatttcttgccttctgctagcttttgaatgtgtttgctcttgcttctctagttcttttaattgtgatgttagggtgtcaattttagatatttcc

SEQ ID NO: 201 - DNA, 401 bp, Homo sapiens
ctagtggtctatcaattttgttgatcttttcaaaaaaccagctcctggattcattgattttttgaagggttttttgtgtctctattttcttcagttctgctctgatcttagttatttcttgccttctgctagcttttgaatgtgtttgctcttgcttctctagttcttttaattgtgatgttagggtgtcaattttagatmtttcctgctttctcttgtgggcatttactgctgtaaatttccgtctacacactgctttgaatgtgtcccagagattctggtatgttgtgtctttgttctcgttggtttcaaagaacatctttatttctgccttcatttcgttatgtacccagtagtcattcaggagcaggttgttcagtttccatgtagttgagcggttt

SEQ ID NO: 202 - DNA, 401 bp, Homo sapiens
cagttctgctctgatcttagttatttcttgccttctgctagcttttgaatgtgtttgctcttgcttctctagttcttttaattgtgatgttagggtgtcaattttagatatttcctgctttctcttgtgggcatttactgctgtaaatttccgtctacacactgctttgaatgtgtcccagagattctggtatgttgtgtstttgttctcgttggtttcaaagaacatctttatttctgccttcatttcgttatgtacccagtagtcattcaggagcaggttgttcagtttccatgtagttgagcggttttgagtgagtttcttaatcctgagttctagtttgattgcactgtggtctgagagacagtttgttataatttctgttcttttacattagctga

SEQ ID NO: 203 - DNA, 401 bp, Homo sapiens
tcattcccctccctcccttccattcctttcccagtctttaatccccctgttccttcccttctcctactcccttaatcacttacagtcccactcccaggaggaaggggtacaggaaactgcatttctcctcctgaaggtcactgtgtgcagctgaagccacttagtccacaggaaggaaggccagctggtaggagttcagayggcagttacagccaccaggggacagctttggcatccacccccttcagggctctcctgagagggaggagcagtgcccaccctagggagggctgagctttatcacactctctgagggctctctgggtcctgcctcatcagctctgatggcggaagggttgacagggggcagcaaggatggttttgtcctgtggcacctcacc

SEQ ID NO: 204 - DNA, 401 bp, Homo sapiens
cattcttatacaccaataacagacaaacagagagccaaatcatgagtgaactcccatttgccattgcttcacagagaataaaatacctaggaatccaacttataagggatatgaaggacctcttcaaggagaactacaaaccactgctcaacaaaacaaaagaggacacaaacaaatggaagaacattccatgctcatggrtaggaagaatcaatatcgtgaaaatggccacactgtccaaggtaatttacagattcaatgccatccccatcaagctaccaatgactttcttcacagaattggaaaaaactactttaaagttcatatggaaccaaaaaaagggccctcattgccaagtcaatcctaagccaaaagaacaaagctggaggcatcacactacc

SEQ ID NO: 205 - DNA, 401 bp, Homo sapiens
tatataggcagccaccctgaagccagagacatggagtgcctgtctcaagtcacctgaaatccaagacacagcactgatttgaacttctggcttctggcaccacaggccccaagcgcagctcttcctcctccacaggcctggaggctctgcacacctgcttcctgccttccaggggagactgtacctaagatgctttcagcrtctattctgtcccgagcatgggccagagagagtagcagacactcacctgtcgggagaacccacgccaagcaggccagcagccagatggacttttccatgctgtgagcatctgaacaccccaggccagacccgctctcggccacctccccagtgagggccctcccaggcgtggtgggggaaggaaggcgggcgggggtggg

SEQ ID NO: 206 - DNA, 401 bp, Homo sapiens
ccctcccatctggcgtgtactcagactggagtccagctcggggctggtttgggcagtaacagcaaacctctgcacagctgtcaccacatgctgggcactttagtaactcacataaggctgaaaaccctatgagaaaggaactctgtttgtttgtttgtttctctttggagacagagtcccactctgtcacccaggctggartgcagtggcgcaatctcagctcactgtaaactctgcctcccaagttcaagtgattcttgtgcctcagcctcccaagtagctgggattacaagtgtgtgccaccacgcccagctaattttttaaatttttagtagagacagaattttgccacgttgtccagcctggtctcgaactcctgacctcaagtgatcagcccgcct

SEQ ID NO: 207 - DNA, 401 bp, Homo sapiens
cacatacctacaggtgaggtcgacagccaatgggagtgtctctccttgccctcaaaatgccctctcctggccctccaggtgtcacaatgccggaagtgccacccccggagccacactcctggagcaccctgttagcatgcccaggtgccctcaaggagggtagcccctcccagattgttgggctcctcatactggccactygtcagctggtaatgtcagccatgtggtggtggcaggggcacaaaagatgcctgagggactccagctctgagggccacaatgctgtccctcctggaggcgtggtcttcctatggtgactgcccacttggttccatccttcccttaccccctcaaaatccgtgtcacataaagcccaccccagcagacctaatttttgttaa

SEQ ID NO: 208 - DNA, 401 bp, Homo sapiens
ccagagagcaagccccactagagtgtggacgtaagacagggacagtaggggcagcccgggagaggaaaaggcaagtagaaagactcaggtgaggcccagaaaggaggcaggcatttctggagggtggacgggaggaaggcaggaaaggcgtgggggttattgtgggtgcaatgggaagtcactgaatggttttaagcggaygcatggctccatcagaacaagctggcccaggagacttggttgcaggccgttggcaaggctgaggctggcttggctgtttggacaaaggtgcaggggtggcggtggggaagtgggtagacgggaggcagggcttgcccaagagcccagtgggcagcacacagtgtgagagggagccaggctgccggggttgggagcctgag

SEQ ID NO: 209 - protein, 389 aa, Homo sapiens
MEDSPTMVRVDSPTMVRGENQVSPCQGRRCFPKALGYVTGDMKELANQLKDKPVVLQFIDWILRGISQVVFVNNPVSGILILVGLLVQNPWWALTGWLGTVVSTLMALLLSQDRSLIASGLYGYNATLVGVLMAVFSDKGDYFWWLLLPVCAMSMTCPIFSSALNSMLSKWDLPVFTLPFNMALSMYLSATGHYNPFFPAKLVIPITTAPNISWSDLSALELLKSIPVGVGQIYGCDNPWTGGIFLGAILLSSPLMCLHAAIGSLLGIAAGLSLSAPFEDIYFGLWGFNSSLACIAMGGMFMALTWQTHLLALGCALFTAYLGVGMANFMAEVGLPACTWPFCLATLLFLIMTTKNSNIYKMPLSKVTYPEENRIFYLQAKKRMVESPL

(19) United States
(12) Patent Application Publication
(10) Pub. No.: US 2013/0296175 A1
(43) Pub. Date: Nov. 7, 2013

(54) Genetic Variants as Markers for Use in Urinary Bladder Cancer Risk Assessment, Diagnosis, Prognosis and Treatment

(71) Applicants: Thorunn Rafnar, Reykjavik (IS); Patrick Sulem, Reykjavik (IS)

(72) Inventors: Thorunn Rafnar, Reykjavik (IS); Patrick Sulem, Reykjavik (IS)

(73) Assignees: ILLUMINA INC., San Diego, CA (US); deCODE Genetics ehf., Reykjavik (IS)

(21) Appl. No.: 13/979,119

(22) PCT Filed: Jan. 12, 2012
(86) PCT No.: PCT/IS2012/050001

§ 371 Date: Jul. 10, 2013

(30) Foreign Application Priority Data
Jan. 13, 2011 (IS) 050004

(51) Int. Cl. C12Q 1/68; G06F 19/18; G01N 33/68

(52) U.S. Cl. CPC C12Q 1/6886; G01N 33/6872; G06F 19/18
USPC 506/2; 435/6.11; 435/7.23; 435/4; 506/9; 506/16; 702/20

(57) ABSTRACT

Polymorphic variants that have been found to be associated with risk of urinary bladder cancer are provided herein. Such polymorphic markers are useful for diagnostic purposes, such as in methods of determining a susceptibility, and for prognostic purposes, including methods of predicting prognosis and methods of assessing an individual for probability of a response to therapeutic 5 agents, as further described herein. Further applications utilize the polymorphic markers of the invention include screening and genotyping methods. The invention furthermore provides related kits, and computer-readable media and apparatus.

### BACKGROUND OF THE INVENTION

Urinary bladder cancer (UBC) is the 6th most common type of cancer in the United States with approximately 67,000 new cases and 14,000 deaths from the disease in 2007. UBC tends to occur most commonly in individuals over 60 years of age. Exposure to certain industrially used chemicals (derivatives of compounds called arylamines) is strong risk factor for the development of bladder cancers. Tobacco use (specifically cigarette smoking) is thought to cause 50% of bladder cancers discovered in male patients and 30% of those found in female patients. Thirty percent of bladder tumors probably result from occupational exposure in the workplace to carcinogens such as benzidine. Occupations at risk are metal industry workers, rubber industry workers, workers in the textile industry and people who work in printing. Certain drugs such as cyclophosphamide and phenacetin are known to predispose to bladder cancer. Chronic bladder irritation (infection, bladder stones, catheters, and bilharzia) predisposes to squamous cell carcinoma of the bladder.

Familial clustering of UBC cases suggests that there is a genetic component to the risk of the disease (Aben, K. K. et al. “Familial aggregation of urothelial cell carcinoma”. Int J Cancer 98, 274-8 (2002); Amundadottir, L. T. et al. “Cancer as a Complex Phenotype: Pattern of Cancer Distribution within and beyond the Nuclear Family.” PLoS Med 1, e65 Epub 2004 Dec. 28 (2004); Murta-Nascimento, C. et al. “Risk of bladder cancer associated with family history of cancer: do low-penetrance polymorphisms account for the increase in risk?” Cancer Epidemiol Biomarkers Prev 16, 1595-600 (2007)). Genetic segregation analyses have suggested that this component is multifactorial with many genes conferring small risks (Aben, K. K. et al. “Segregation analysis of urothelial cell carcinoma.” Eur J Cancer 42, 1428-33 (2006)). Many epidemiological studies have evaluated potential associations between sequence variants in candidate genes and bladder cancer, but the most consistent risk association to the disease is found for variations in the NAT2 gene. (Sanderson, S. et al., “Joint effects of the N-acetyltransferase 1 and 2 (NAT1 and NAT2) genes and smoking on bladder carcinogenesis: a literature-based systematic HuGE review and evidence synthesis.” Am J Epidemiol 166, 741-51 (2007)).

Majority (>90%) of bladder cancers are transitional cell carcinomas (TCC) and arise from the urothelium. Other bladder cancer types include squamous cell carcinoma, adenocarcinoma, sarcoma, small cell carcinoma and secondary deposits from cancers elsewhere in the body. TCCs are often multifocal, with 30-40% of patients having a more than one tumor at diagnosis. The pattern of growth of TCCs can be papillary, sessile (flat) or carcinoma-in-situ (CIS). Superficial tumors are defined as tumors that either do not invade, or those that invade but stay superficial to the deep muscle wall of the bladder. At initial diagnosis, 70% of patients with bladder cancers have superficial disease. Tumors that are clinically superficial are composed of three distinctive pathologic types. The majority of superficial urothelial carcinomas present as noninvasive, papillary tumors (pathologic stage pTa (or Ta)). 70% of these superficial papillary tumors will recur over a prolonged clinical course, causing significant morbidity. In addition, 5-10% of these papillary lesions will eventually progress to invasive carcinomas. These tumors are pathologically graded as either low malignant potential, low grade or high grade. High grade tumors have a higher risk of progression. Flat urothelial carcinoma in situ (CIS) are highly aggressive lesions and progress more rapidly than the papillary tumors. A minority of tumors invade only superficially into the lamina propria. These tumors recur 80% of the time, and eventually invade the detrusor muscle in 30% of cases. Approximately 30% of urothelial carcinomas invade the detrusor muscle at presentation. These cancers are highly aggressive. Those invasive tumors may spread by way of the lymph and blood systems to invade bone, liver, and lungs and have high morbidity (Kaufman, D. S. Ann Oncol 17, v106-112 (2006)).

The treatment of transitional cell or urothelial carcinoma is different for superficial tumors and muscle invasive tumors. Superficial bladder cancers can be managed without cystectomy (removing the bladder). The standard initial treatment of superficial tumors includes cystoscopy with trans-urethral resection of the tumor (TUR). The cystoscope allows visualization and entire removal of a bladder tumor. Adjuvant intravesical drug therapy after TUR is commonly prescribed for patients with tumors that are large, multiple, high grade or superficially invasive. Intravesical therapy consists of drugs placed directly into the bladder through a urethral catheter, in an attempt to minimize the risk of tumor recurrence and progression. About 50-70% of patients with superficial bladder cancer have a very good response to intravesical therapy. The current standard of care consists of urethro-cystoscopy and urine cytology every 3-4 months for the first two years and at a longer interval in subsequent years.

Cystectomy is indicated when bladder cancer is invasive into the muscle wall of the bladder or when patients with superficial tumors have frequent recurrences that are not responsive to intravesical therapy. The benefits of surgically removing the bladder are disease control, eradication of symptoms associated with bladder cancer, and long-term survival. For advanced bladder cancer that has extended beyond the bladder wall, radiation and chemotherapy are treatment options. Local lymph nodes are frequently radiated as part of the therapy to treat the microscopic cancer cells which may have spread to the nodes. Current treatment of advanced bladder cancer can involve a combination of radiation and chemotherapy.

Early detection can improve prognosis, treatment options as well as quality of life of the patient. If screening methods could detect bladder cancers destined to become muscle invading while they are still superficial it is likely that a significant reduction in morbidity and mortality would result.

Cystoscopic examination is costly and causes substantial discomfort for the patient. Urine cytology has poor sensitivity in detecting low-grade disease and its accuracy can vary between pathology labs. Many urine-based tumor markers have been developed for detection and surveillance of the disease and some of these are used in routine patient care (Lokeshwar, V. B. et al. Urology 66, 35-63 (2005); Friedrich, M. G. et al. 133U Int 92, 389-92 (2003); Ramakumar, S. et al. J Urol 161, 388-94 (1999); Sozen, S. et al. Eur Urol 36, 225-9 (1999); Heicappell, R. et al. Urol Int 65, 181-4 (2000)).

However, no biomarker reported to date has shown sufficient sensitivity and specificity for detecting all types of bladder cancers in the clinic. It should be remembered that efficiency of screening increases with the disease's prevalence in the screened population. Therefore, the efficiency of the test could be increased by limiting the screening program to people at high risk. For bladder cancer, this may mean restricting participation to people with occupational exposure to known bladder carcinogens or individuals with known cancer predisposing variants.

There is clearly a need for improved diagnostic procedures that would facilitate early-stage bladder cancer detection and prognosis, as well as aid in preventive and curative treatments of the disease. In addition, there is a need to develop tools to better identify those patients who are more likely to have aggressive forms of bladder cancer from those patients that are diagnosed with the superficial disease. This would help to avoid invasive and costly procedures for patients not at significant risk.

Genetic risk is conferred by subtle differences in the genome among individuals in a population. Variations in the human genome are most frequently due to single nucleotide polymorphisms (SNPs), although other variations are also important. SNPs are located on average every 1000 base pairs in the human genome. Accordingly, a typical human gene containing 250,000 base pairs may contain 250 different SNPs. Only a minor number of SNPs are located in exons and alter the amino acid sequence of the protein encoded by the gene. Most SNPs may have little or no effect on gene function, while others may alter transcription, splicing, translation, or stability of the mRNA encoded by the gene. Additional genetic polymorphisms in the human genome are caused by insertions, deletions, translocations or inversion of either short or long stretches of DNA. Genetic polymorphisms conferring disease risk may directly alter the amino acid sequence of proteins, may increase the amount of protein produced from the gene, or may decrease the amount of protein produced by the gene.

As genetic polymorphisms conferring risk of common diseases are uncovered, genetic testing for such risk factors is becoming increasingly important for clinical medicine. Examples are apolipoprotein E testing to identify genetic carriers of the apoE4 polymorphism in dementia patients for the differential diagnosis of Alzheimer's disease, and of Factor V Leiden testing for predisposition to deep venous thrombosis. More importantly, in the treatment of cancer, diagnosis of genetic variants in tumor cells is used for the selection of the most appropriate treatment regime for the individual patient. In breast cancer, genetic variation in estrogen receptor expression or heregulin type 2 (Her2) receptor tyrosine kinase expression determine if anti-estrogenic drugs (tamoxifen) or anti-Her2 antibody (Herceptin) will be incorporated into the treatment plan. In chronic myeloid leukemia (CML) diagnosis of the Philadelphia chromosome genetic translocation fusing the genes encoding the Bcr and Abl receptor tyrosine kinases indicates that Gleevec (STI571), a specific inhibitor of the Bcr-Abl kinase should be used for treatment of the cancer. For CML patients with such a genetic alteration, inhibition of the Bcr-Abl kinase leads to rapid elimination of the tumor cells and remission from leukemia. Furthermore, genetic testing services are now available, providing individuals with information about their disease risk based on the discovery that certain SNPs have been associated with risk of many of the common diseases.

Loci Associated with Bladder Cancer

The genetic polymorphisms in a number of metabolic enzymes and other genes have been found as the modulators of bladder cancer risk. The most studied polymorphisms in connection with bladder cancer risk are polymorphisms in genes for some important enzymes, especially N-acetyl-transferases (NATs), glutathione S-transferases (GSTs), DNA repair enzymes, and many others. An improved understanding of the molecular biology of urothelial malignancies is helping to define more clearly the role of new prognostic indices and multidisciplinary treatment for this disease.

It has been suggested that some of the NAT variants modify individual susceptibility to cancer. Slow NAT2 acetylation capacity has been suggested as conferring an increased risk of bladder, breast, liver and lung cancers, and a decreased risk of colon cancer, whereas a prominent change in the NAT1 gene, putatively associated with increased NAT1 activity, has been suggested as increasing the risk of bladder and colon cancer, and decreasing that of lung cancer (A. Hirvonen, IARC Sci Publ 148 (1999), pp. 251-270). NAT1 polymorphisms may affect the individual bladder cancer risk by interacting with environmental factors and interacting with the NAT2 gene (Cascorbi I, et al. Cancer Res 61:5051-6).

Glutathione S-transferases (GST) comprise a major group of enzymes that play a key role in detoxification of carcinogenic compounds. At least five GST families have been identified, and the effects of polymorphisms in these genes have been studied in bladder cancer. The results from these studies are contradictory but association between GSTM1 null genotype and bladder cancer is fairly constant (Wu, X. et al. Front Biosci 12, 192-213 (2007)).

Polymorphisms in genes coding for other metabolic enzymes such as NQO1, MPO or the CYP enzyme superfamily have also in some studies been found to be associated with bladder cancer but the results are controversial (Wu, X. et al. supra). Since bladder cancer has strong environmental risk factors, polymorphisms in DNA repair genes have been studied in bladder cancer patients. These include genes for Xeroderma pigmentosum (XP) and X-ray repair cross-complementing (XRCC) genes. Many different polymorphisms have been tested but larger sample size and better matching between cases and controls is needed to conclude the effects of these variants on bladder cancer risks.

Recently performed genome-wide association studies of UBC have resulted in the identification of genetic variants associated with UBC in several distinct locations (Kiemeney, L A, et al. Nat Genet. 40:1307-12 (2008); Wu, X. et al. Nat Genet. 41:991-5 (2009); Rafnar, T. et al. Nat Genet. 41:221-7 (2009) and Kiemeney, L A, et al. Nat Genet. 42:415-419 (2010)). These loci, however, only explain a portion of the genetic risk of UBC in the human population. Thus, it is clear that additional genetic risk factors for UBC remain to be found. It is likely that these genetic risk factors will include a relatively high number of low-to-medium risk genetic variants. These low-to-medium risk genetic variants may, however, be responsible for a substantial fraction of bladder cancer, and their identification, therefore, a great benefit for public health. The present invention provides such additional genetic risk factors of UBC.

### SUMMARY OF THE INVENTION

The present invention is based on the finding that certain genetic variants are associated with risk of urinary bladder cancer (UBC). The invention provides diagnostic applications based on this surprising finding, including methods, kits, media and apparati useful for determining UBC risk.

In a first aspect, the invention provides a method of determining a susceptibility to urinary bladder cancer in a human individual, the method comprising analyzing nucleic acid sequence data from a human individual for at least one polymorphic marker in the human SLC14A1 gene, wherein different alleles of the at least one polymorphic marker are associated with different susceptibilities to Bladder Cancer in humans, and determining a susceptibility to Bladder Cancer from the nucleic acid sequence data. In one embodiment, the nucleic acid sequence data is obtained from a biological sample containing nucleic acid from the human individual.

In another aspect, the invention provides a method of determining a susceptibility to Bladder Cancer, the method comprising obtaining amino acid sequence data about at least one encoded SLC14A1 protein in a human individual, and analyzing the amino acid sequence data to determine whether at least one amino acid substitution predictive of increased susceptibility of Bladder Cancer is present, wherein a determination of the presence of the at least one amino acid substitution is indicative of increased susceptibility of Bladder Cancer for the individual, and wherein a determination of the absence of the at least one amino acid substitution is indicative of the individual not having the increased susceptibility.

As a consequence of the foregoing, the invention in another aspect provides a method of determining a susceptibility to Bladder Cancer, the method comprising analyzing amino acid sequence data about at least one encoded SLC14A1 protein in a human individual, and/or nucleic acid sequence data about at least one polymorphic marker in the human SLC14A1 gene, wherein different alleles of the at least one polymorphic marker and/or at least one amino acid substitution are associated with different susceptibilities to Bladder Cancer in humans, and determining a susceptibility to Bladder Cancer from the nucleic acid sequence data and/or the amino acid sequence data.

The invention further provides a method of identification of a marker for use in assessing susceptibility to urinary bladder cancer in human individuals, the method comprising (a) identifying at least one polymorphic marker in the human SLC14A1 gene; (b) obtaining sequence information about the at least one polymorphic marker in a group of individuals diagnosed with urinary bladder cancer; and (c) obtaining sequence information about the at least one polymorphic marker in a group of control individuals; wherein determination of a significant difference in frequency of at least one allele in the at least one polymorphism in individuals diagnosed with urinary bladder cancer as compared with the frequency of the at least one allele in the control group is indicative of the at least one polymorphism being useful for assessing susceptibility to urinary bladder cancer. In one embodiment, an increase in frequency of the at least one allele in the at least one polymorphism in individuals diagnosed with urinary bladder cancer, as compared with the frequency of the at least one allele in the control group, is indicative of the at least one polymorphism being useful for assessing increased susceptibility to urinary bladder cancer, and wherein a decrease in frequency of the at least one allele in the at least one polymorphism in individuals diagnosed with urinary bladder cancer, as compared with the frequency of the at least one allele in the control group, is indicative of the at least one polymorphism being useful for assessing decreased susceptibility to, or protection against, urinary bladder cancer.

The invention further provides a method of assessing a subject's risk for urinary bladder cancer, the method comprising (a) obtaining sequence information about the individual identifying at least one allele of at least one polymorphic marker in the genome of the individual; (b) representing the sequence information as digital genetic profile data; (c) transforming the digital genetic profile data on a computer processor to generate risk assessment report of urinary bladder cancer for the subject; and (d) displaying the risk assessment report on an output device; wherein the at least one polymorphic marker is a marker within the human SLC14A1 gene that is predictive of risk of urinary bladder cancer in humans.

Further provided is a method of determining whether an individual is at increased risk of developing bladder cancer, the method comprising steps of (i) obtaining a biological sample containing nucleic acid from the individual; (ii) determining, in the biological sample, nucleic acid sequence about the SLC14A1 gene; and (iii) comparing the sequence information to the wild-type nucleic acid sequence of SLC14A1 (SEQ ID NO:134); wherein an identification of a mutation in SLC14A1 in the individual is indicative that the individual is at increased risk of developing bladder cancer.

The invention further provides a method of determining the recurrence risk of an individual diagnosed with urinary bladder cancer, the method comprising steps of (a) obtaining sequence data about a human individual who has been diagnosed with urinary bladder cancer, identifying at least one allele of at least one polymorphic marker, wherein different alleles of the at least one polymorphic marker are associated with different recurrence risk of urinary bladder cancer in humans, and (b) determining the recurrence risk of urinary bladder cancer for the human individual from the sequence data; wherein the at least one polymorphic marker is a marker in the human SLC14A1 gene, wherein different alleles of the at least one polymorphic marker are associated with different recurrence risk of urinary bladder cancer in humans.

Further provided is a method of predicting prognosis of an individual diagnosed with urinary bladder cancer, the method comprising obtaining sequence data about a human individual identifying at least one allele of at least one polymorphic marker in the human SLC14A1 gene, wherein different alleles of the at least one polymorphic marker are predictive of different prognosis of urinary bladder cancer in humans, and predicting prognosis of urinary bladder cancer from the sequence data.

It may be useful to be able to determine which individuals are suitably for further diagnostic evaluation of urinary bladder cancer. The present invention thus also provides a method for identifying a subject who is a candidate for further diagnostic evaluation for urinary bladder cancer, comprising the steps of (a) determining, in the genome of a human subject, the allelic identity of at least one polymorphic marker in the human SLC14A1 gene, and/or the identity of at least one amino acid at a variant amino acid position in an encoded SLC14A1 protein, wherein different alleles of the at least one marker and/or the identity of the at least one amino acid are associated with different susceptibilities to urinary bladder cancer in humans; and (b) identifying the subject as a subject who is a candidate for further diagnostic evaluation for urinary bladder cancer based on the allelic identity at the at least one polymorphic marker and/or the identity of the at least one amino acid. In a preferred embodiment, the further diagnostic evaluation comprises urine cytology, cystoscopy and/or a Hematuria test.

Assessment of genetic risk can be reported in a risk assessment report. The invention therefore also provides in one aspect a risk assessment report comprising (a) at least one personal identifier, and (b) representation of at least one risk assessment measure of urinary bladder cancer for the human subject for at least one polymorphic marker or at least one amino acid variation.

Kits are also provided. In one embodiment, a kit for assessing susceptibility to urinary bladder cancer in humans is provided, the kit comprising reagents for selectively detecting at least one at-risk variant for Bladder Cancer in the individual, wherein the at least one risk variant is a marker in the human SLC14A1 gene or an amino acid variant in an encoded SLC14A1 protein, and a collection of data comprising correlation data between the at least one marker and susceptibility to urinary bladder cancer. The at least one marker is in one embodiment selected from the group consisting of rs1058396, and markers in linkage disequilibrium therewith.

The present invention also provides diagnostic reagents. In one such aspect, the invention relates to the use of an oligonucleotide probe in the manufacture of a diagnostic reagent for diagnosing and/or assessing a susceptibility to urinary bladder cancer in humans, wherein the probe is capable of hybridizing to a segment of the human SLC14A1 gene with sequence as given by SEQ ID NO:134, and wherein the segment is 15-400 nucleotides in length. In a suitable embodiment, the segment of the nucleic acid to which the probe is capable of hybridizing comprises a polymorphic site. The polymorphic site is suitably selected from the group consisting of the markers rs1058396, rs11877062, rs2298720, rs2298719, and markers in linkage disequilibrium therewith.

The invention also provides computer-implemented aspects. As is known in the art, sequence data can conveniently be stored and analyzed in digital format, and either such sequence data (e.g., genotype data) or results derived therefrom (e.g., disease-risk estimates) can be provided in digital format to an end-user.

One such aspect relates to a computer-readable medium having computer executable instructions for determining susceptibility to urinary bladder cancer in humans, the computer readable medium comprising (i) sequence data identifying at least one allele of at least one polymorphic marker in the individual; and (ii) a routine stored on the computer readable medium and adapted to be executed by a processor to determine risk of developing Bladder Cancer for the at least one polymorphic marker; wherein the at least one polymorphic marker is a marker in the human SLC14A1 gene, or an amino acid variant in an encoded SLC14A1 protein, that is predictive of susceptibility of Bladder Cancer in humans.

Another computer-implemented aspect relates to an apparatus for determining a genetic indicator for urinary bladder cancer in a human individual, comprising (i) a processor; and (ii) a computer readable memory having computer executable instructions adapted to be executed on the processor to analyze marker information for at least one marker in the human SLC14A1 gene that is predictive of susceptibility to Bladder Cancer in humans, or at least one amino acid variation in an encoded SLC14A1 protein, and generate an output based on the marker or amino acid information, wherein the output comprises at least one measure of susceptibility to Bladder Cancer for the human individual.

In one embodiment, the computer readable memory further comprises data indicative of the risk of developing urinary bladder cancer associated with at least one allele of at least one polymorphic marker, and wherein a risk measure for the human individual is based on a comparison of the marker information for the human individual to the risk of urinary bladder cancer associated with the at least one allele of the at least one polymorphic marker.

In certain embodiments, the polymorphic marker is suitably selected from the group consisting of the markers rs1058396, rs11877062, rs2298720, rs2298719, and markers in linkage disequilibrium therewith. In certain embodiments, the amino acid variation is selected from the group consisting of an asparagine to aspartic acid substitution at position 336, an arginine to tryptophan substitution at position 4, a lysine to glutamic acid substitution at position 100 and a methionine to valine substitution at position 223, all in a protein with sequence as set forth in SEQ ID NO:133. The invention also provides risk assessment reports. One such aspect relates to a risk assessment report of urinary bladder cancer for a human individual, comprising (i) at least one personal identifier, and (ii) representation of at least one risk assessment measure of urinary bladder cancer for the human subject for at least one polymorphic marker in the human SLC14A1 gene, wherein different alleles of the at least one polymorphic marker are associated with different susceptibilities to Bladder Cancer in humans. Such reports may be provided in any suitable format, including electronic format (e.g., on a computer-readable medium) or a paper format (e.g., a reported printed or written on paper).

A further aspect of the invention is to provide use of variants for selecting individuals for administration of therapeutic agents for treating urinary bladder cancer. One such aspect provides use of an agent for treating urinary bladder cancer in a human individual that has been tested for the presence of at least one allele of at least one risk marker of urinary bladder cancer, as described herein.

It should be understood that all combinations of features described herein are contemplated, even if the combination of feature is not specifically found in the same sentence or paragraph herein. This includes in particular the use of all markers disclosed herein, alone or in combination, for analysis individually or in haplotypes, in all aspects of the invention as described herein.

### BRIEF DESCRIPTION OF THE DRAWINGS

The foregoing and other objects, features and advantages of the invention will be apparent from the following more particular description of preferred embodiments of the invention.

FIG. 1 shows a schematic view of an exemplary computer system for implementing the invention.

FIG. 2 shows a diagram illustrating a system comprising computer implemented methods utilizing risk variants as described herein.

FIG. 3 shows an exemplary system for determining risk of cancer as described further herein.

FIG. 4 shows a system for selecting a treatment protocol for a subject diagnosed with a cancer.

### DETAILED DESCRIPTION

### Definitions

Unless otherwise indicated, nucleic acid sequences are written left to right in a 5′ to 3′ orientation. Numeric ranges recited within the specification are inclusive of the numbers defining the range and include each integer or any non-integer fraction within the defined range. Unless defined otherwise, all technical and scientific terms used herein have the same meaning as commonly understood by the ordinary person skilled in the art to which the invention pertains.

The following terms shall, in the present context, have the meaning as indicated:

A “polymorphic marker”, sometime referred to as a “marker”, as described herein, refers to a genomic polymorphic site. Each polymorphic marker has at least two sequence variations characteristic of particular alleles at the polymorphic site. Thus, genetic association to a polymorphic marker implies that there is association to at least one specific allele of that particular polymorphic marker. The marker can comprise any allele of any variant type found in the genome, including SNPs, mini- or microsatellites, translocations and copy number variations (insertions, deletions, duplications). Polymorphic markers can be of any measurable frequency in the population. For mapping of disease genes, polymorphic markers with population frequency higher than 5-10% are in general most useful. However, polymorphic markers may also have lower population frequencies, such as 1-5% frequency, or even lower frequency, in particular copy number variations (CNVs). The term shall, in the present context, be taken to include polymorphic markers with any population frequency.

An “allele” refers to the nucleotide sequence of a given locus (position) on a chromosome. A polymorphic marker allele thus refers to the composition (i.e., sequence) of the marker on a chromosome. Genomic DNA from an individual contains two alleles (e.g., allele-specific sequences) for any given polymorphic marker, representative of each copy of the marker on each chromosome. Sequence codes for nucleotides used herein are: A=1, C=2, G=3, T=4. For microsatellite alleles, the CEPH sample (Centre d'Etudes du Polymorphisme Humain, genomics repository, CEPH sample 1347-02) is used as a reference, the shorter allele of each microsatellite in this sample is set as 0 and all other alleles in other samples are numbered in relation to this reference. Thus, e.g., allele 1 is 1 bp longer than the shorter allele in the CEPH sample, allele 2 is 2 bp longer than the shorter allele in the CEPH sample, allele 3 is 3 bp longer than the lower allele in the CEPH sample, etc., and allele −1 is 1 bp shorter than the shorter allele in the CEPH sample, allele −2 is 2 bp shorter than the shorter allele in the CEPH sample, etc.

Sequence conucleotide ambiguity as described herein is as proposed by IUPAC-IUB. These codes are compatible with the codes used by the EMBL, GenBank, and PIR databases.

| IUB code | Meaning |
|---|---|
| A | Adenosine |
| C | Cytidine |
| G | Guanine |
| T | Thymidine |
| R | G or A |
| Y | T or C |
| K | G or T |
| M | A or C |
| S | G or C |
| W | A or T |
| B | C, G or T |
| D | A, G or T |
| H | A, C or T |
| V | A, C or G |
| N | A, C, G or T (Any base) |

A nucleotide position at which more than one sequence is possible in a population (either a natural population or a synthetic population, e.g., a library of synthetic molecules) is referred to herein as a “polymorphic site”.

A “Single Nucleotide Polymorphism” or “SNP” is a DNA sequence variation occurring when a single nucleotide at a specific location in the genome differs between members of a species or between paired chromosomes in an individual. Most SNP polymorphisms have two alleles. Each individual is in this instance either homozygous for one allele of the polymorphism (i.e. both chromosomal copies of the individual have the same nucleotide at the SNP location), or the individual is heterozygous (i.e. the two sister chromosomes of the individual contain different nucleotides). The SNP nomenclature as reported herein refers to the official Reference SNP (rs) ID identification tag as assigned to each unique SNP by the National Center for Biotechnological Information (NCBI).

A “variant”, as described herein, refers to a segment of DNA that differs from the reference DNA. A “marker” or a “polymorphic marker”, as defined herein, is a variant. Alleles that differ from the reference are referred to as “variant” alleles.

A “microsatellite” is a polymorphic marker that has multiple small repeats of bases that are 2-8 nucleotides in length (such as CA repeats) at a particular site, in which the number of repeat lengths varies in the general population. An “indel” is a common form of polymorphism comprising a small insertion or deletion that is typically only a few nucleotides long.

A “haplotype,” as described herein, refers to a segment of genomic DNA that is characterized by a specific combination of alleles arranged along the segment. For diploid organisms such as humans, a haplotype comprises one member of the pair of alleles for each polymorphic marker or locus along the segment. In a certain embodiment, the haplotype can comprise two or more alleles, three or more alleles, four or more alleles, or five or more alleles.

Allelic identities are described herein in the context of the marker name and the particular allele of the marker, e.g., “1 rs1058396” refers to the 1 allele of marker rs1058396, and is equivalent to “rs1058396 allele 1”. Furthermore, allelic codes are as for individual markers, i.e. 1=A, 2=C, 3=G and 4=T.

The term “susceptibility”, as described herein, refers to the proneness of an individual towards the development of a certain state (e.g., a certain trait, phenotype or disease), or towards being less able to resist a particular state than the average individual. The term encompasses both increased susceptibility and decreased susceptibility. Thus, particular alleles at polymorphic markers and/or haplotypes comprising such markers, including those described herein, may be characteristic of increased susceptibility (i.e., increased risk) of urinary bladder cancer, as characterized by a relative risk (RR) or odds ratio (OR) of greater than one for the particular allele or haplotype. Alternatively, the markers and/or haplotypes of the invention are characteristic of decreased susceptibility (i.e., decreased risk) of urinary bladder cancer, as characterized by a relative risk of less than one.

The term “and/or” shall in the present context be understood to indicate that either or both of the items connected by it are involved. In other words, the term herein shall be taken to mean “one or the other or both”.

The term “look-up table”, as described herein, is a table that correlates one form of data to another form, or one or more forms of data to a predicted outcome to which the data is relevant, such as phenotype or trait. For example, a look-up table can comprise a correlation between allelic data for at least one polymorphic marker and a particular trait or phenotype, such as a particular disease diagnosis, that an individual who comprises the particular allelic data is likely to display, or is more likely to display than individuals who do not comprise the particular allelic data. Look-up tables can be multidimensional, i.e. they can contain information about multiple alleles for single markers simultaneously, or they can contain information about multiple markers, and they may also comprise other factors, such as particulars about diseases diagnoses, racial information, biomarkers, biochemical measurements, therapeutic methods or drugs, etc.

A “computer-readable medium”, is an information storage medium that can be accessed by a computer using a commercially available or custom-made interface. Exemplary computer-readable media include memory (e.g., RAM, ROM, flash memory, etc.), optical storage media (e.g., CD-ROM), magnetic storage media (e.g., computer hard drives, floppy disks, etc.), punch cards, or other commercially available media. Information may be transferred between a system of interest and a medium, between computers, or between computers and the computer-readable medium for storage or access of stored information. Such transmission can be electrical, or by other available methods, such as IR links, wireless connections, etc.

A “nucleic acid sample” as described herein, refers to a sample obtained from an individual that contains nucleic acid (DNA or RNA). In certain embodiments, i.e. the detection of specific polymorphic markers and/or haplotypes, the nucleic acid sample comprises genomic DNA. Such a nucleic acid sample can be obtained from any source that contains genomic DNA, including a blood sample, sample of amniotic fluid, sample of cerebrospinal fluid, or tissue sample from skin, muscle, buccal or conjunctival mucosa, placenta, gastrointestinal tract or other organs.

The term “UBC therapeutic agent” refers to an agent that can be used to ameliorate or prevent symptoms associated with urinary bladder cancer (UBC).

The term “UBC-associated nucleic acid”, as described herein, refers to a nucleic acid that has been found to be associated to urinary bladder cancer. This includes, but is not limited to, the markers and haplotypes described herein and markers and haplotypes in strong linkage disequilibrium (LD) therewith.

The term “antisense agent” or “antisense oligonucleotide” refers, as described herein, to molecules, or compositions comprising molecules, which include a sequence of purine and pyrimidine heterocyclic bases, supported by a backbone, which are effective to hydrogen bond to a corresponding contiguous bases in a target nucleic acid sequence. The backbone is composed of subunit backbone moieties supporting the purine and pyrimidine heterocyclic bases at positions which allow such hydrogen bonding. These backbone moieties are cyclic moieties of 5 to 7 atoms in size, linked together by phosphorous-containing linkage units of one to three atoms in length. In certain preferred embodiments, the antisense agent comprises an oligonucleotide molecule.

The term “SLC14A1”, as described herein, refers to the Solute Carrier Family 14 (urea transporter), member 1 gene on chromosome 18q12 (pos 41,558,113-41,586,483 in NCBI Build 36 of the human genome assembly). The sequence of the coding region of the gene (Accession No. NM_{—}001128588) is set forth in SEQ ID NO:134.

### Variants Conferring Risk of Urinary Bladder Cancer

The present inventors have found that a non-synonymous polymorphic marker rs1058396 (D280N) in the SLC14A gene on chromosome 18q12.3 is predictive of risk of Urinary Bladder Cancer. The A allele of rs1058396 is associated with risk of Bladder cancer with an OR value of 0.87 (95% Cl; 0.81-0.93) and a P-value of 1.3×10⁻⁴. Thus, the alternate G allele of rs1058396, which encodes an Aspartic acid (D) at position 280 in the encoded protein (splice variant 1 a shown in SEQ ID NO:209; position 336 in splice variant 2 as shown in SEQ ID NO:133), is an at-risk allele for Bladder Cancer. The association was replicated in sample sets from the UK, Italy, Belgium, Sweden, Germany and Eastern Europe. The results from the combined analysis of the discovery set and the replication sample sets showed an OR for the A allele of rs1058396 of 0.90 (95% Cl; 0.85-0.94), corresponding to an OR of 1.11 for the G allele, and a P-value of 3.7×10⁻⁰⁵.

Association was also noted between UBC and missense mutations within the same gene, SLC14A, namely rs11877062 (R4W) and rs2298720 (K44E in SEQ ID NO:209; E100K in SEQ ID NO:133). A fourth risk variant was identified in Icelandic samples, rs2298719 (M223V in SEQ ID NO:133; M167V in SEQ ID NO:209). For these three additional missense variants, the latter amino acid recited (W, E and V for R4W, K44E and M223V, respectively) denotes the amino acid that correlates with increased risk of bladder cancer.

The SLC14A1 human gene consists of 28393 bases and contains 9 coding Exons. The protein encoded by the SLC14A1 gene is a membrane transporter that mediates urea transport in erythrocytes but also forms the basis for the Kidd blood group system that is responsible for the inherited blood types. Thus, the Kidd blood group antigens (called JK) are the product of the SLC14A1 gene. All four markers, i.e. rs1058396, rs11877062, rs2298719 and rs2298720, are missense variants located within the human SLC14A1 gene. It is possible that these variants affect the physiological function of the SLC14A1 gene product. Thus, these variants, or other missense, nonsense, splice site or truncating variants of SLC14A1 may affect SLC14A1 function. For example, these variants may alter the sequence and thus function of the Kidd blood group antigen, thus affecting the blood group status. Variants in the SLC14A1 gene may also result in JK-null variants that lack expression of the protein on blood cells. Variants in SLC14A may also affect the urea transporting properties of the expressed protein, resulting in impaired capacity of carriers of such variants to concentrate urea in the urine.

### Methods of Determining Susceptibility to Urinary Bladder Cancer

Accordingly, the present invention in one aspect provides a method of determining a susceptibility to urinary bladder cancer in a human individual, the method comprising steps of (a) analyzing nucleic acid sequence data from a human individual for at least one polymorphic marker in the human SLC14A1 gene, wherein different alleles of the at least one polymorphic marker are associated with different susceptibilities to Bladder Cancer in humans, and (b) determining a susceptibility to Bladder Cancer from the nucleic acid sequence data. In certain embodiments, the at least one polymorphic marker is selected from the group consisting of rs1058396, rs11877062, rs2298720 and rs2298719, and markers in linkage disequilibrium therewith. In one preferred embodiments, the at least one polymorphic marker is selected from the group consisting of rs1058396, and markers in linkage disequilibrium therewith. In another preferred embodiments, the at least one polymorphic marker is selected from the group consisting of rs1058396, and markers in linkage disequilibrium therewith, characterized by values of the linkage disequilibrium correlation measure r² of greater than 0.2 to rs1058396.

The G allele of rs1058396, the C allele of rs11877062, the G allele of rs2298720, and the A allele of rs2298720 are indicative of an increased risk of Bladder Cancer in humans. Thus, in certain embodiment, determination of the presence of at least one allele selected from the group consisting of the G allele of rs1058396, the C allele of rs11877062, the G allele of rs2298720, and the A allele of rs2298720 is indicative of increased risk of Bladder Cancer for the individual. Determination of the absence of any one of these alleles is indicative that the individual does not have the increased risk conferred by the allele.

In certain embodiments of the invention, the allele that is detected can be the allele of the complementary strand of DNA, such that the nucleic acid sequence data includes the identification of at least one allele which is complementary to any of the alleles of the polymorphic markers referenced above. For example, the allele that is detected may be the complementary C allele of the at-risk G allele of rs1058396, the complementary G allele of the at-risk C allele of rs11877062, the complementary C allele of the at-risk G allele of rs2298720, or the complementary T allele to the at-risk A allele of rs2298720.

In certain embodiments, the nucleic acid sequence data is obtained from a biological sample containing nucleic acid from the human individual. The nucleic acids sequence may suitably be obtained using a method that comprises at least one procedure selected from (i) amplification of nucleic acid from the biological sample; (ii) hybridization assay using a nucleic acid probe and nucleic acid from the biological sample; (iii) hybridization assay using a nucleic acid probe and nucleic acid obtained by amplification of the biological sample, and (iv) sequencing, in particular high-throughput sequencing. The nucleic acid sequence data may also be obtained from a preexisting record. For example, the preexisting record may comprise a genotype dataset for at least one polymorphic marker. In certain embodiments, the determining comprises comparing the sequence data to a database containing correlation data between the at least one polymorphic marker and susceptibility to Bladder Cancer.

It is contemplated that in certain embodiments of the invention, it may be convenient to prepare a report of results of risk assessment. Thus, certain embodiments of the methods of the invention comprise a further step of preparing a report containing results from the determination, wherein said report is written in a computer readable medium, printed on paper, or displayed on a visual display. In certain embodiments, it may be convenient to report results of susceptibility to at least one entity selected from the group consisting of the individual, a guardian of the individual, a genetic service provider, a physician, a medical organization, and a medical insurer.

Surrogate markers in linkage disequilibrium with particular key markers can in general be selected based on any particular numerical values of the linkage disequilibrium measures D′ and r², as described further herein. For example, markers that are in linkage disequilibrium with rs1058396 are exemplified by the markers listed in Table 1 herein, but the skilled person will appreciate that other markers in linkage disequilibrium with rs1058396 marker may also be used in the diagnostic applications described herein. As appreciated by the skilled person, other markers in linkage disequilibrium with rs1058396, rs11877062, rs2298720 and/or rs2298719, for example from public databases comprising information about SNP markers in the human genome, may also be selected to realize the present invention. Further, as also described in more detail herein, the skilled person will appreciate that since linkage disequilibrium is a continuous measure, certain values of the LD measures D′ and r² may be suitably chosen to define markers that are useful as surrogate markers in LD with the markers described herein. Numeric values of D′ and r² may thus in certain embodiments be used to define marker subsets that fulfill certain numerical cutoff values of D′ and/or r². In one embodiment, markers in linkage disequilibrium with a particular anchor marker (e.g., rs1058396) are in LD with the anchor marker characterized by numerical values of D′ of greater than 0.8 and/or numerical values of r² of greater than 0.2. In one embodiment, markers in linkage disequilibrium with a particular anchor marker are in LD with the anchor marker characterized by numerical values of r² of greater than 0.2. For example, the markers provided in Table 1 provide exemplary markers that fulfill this criterion. In other embodiments, markers in linkage disequilibrium with a particular anchor marker are in LD with the anchor marker characterized by numerical values of r² of greater than 0.3, greater than 0.4, greater than 0.5, greater than 0.6, greater than 0.7, greater than 0.8, greater than 0.9, greater than 0.95. Other numerical values of r² and/or D′ may also be suitably selected to select markers that are in LD with the anchor marker. The stronger the LD, the more similar the association signal and/or the predictive risk by the surrogate marker will be to that of the anchor marker. Markers with values of r²=1 to the anchor marker are perfect surrogates of the anchor marker and will provide identical association and risk prediction data. In one preferred embodiment, surrogate markers of rs1058396 are those markers that have values of r² to rs1058396 of greater than 0.2. In another preferred embodiment, surrogate markers of rs1058396 are those markers that have values of r² to rs1058396 of greater than 0.5. In another preferred embodiment, surrogate markers of rs1058396 are those markers that have values of r² to rs1058396 of greater than 0.8 Further, as described in more detail in the following, LD may be determined in samples from any particular population. In one embodiment, LD is determined in Caucasian samples. In another embodiment, LD is determined in European samples. In another embodiment, LD is determined in Icelandic samples. In other embodiments, LD is determined in African American samples, in Asian samples, or the LD may be suitably determined in samples of any other population.

TABLE 1
Surrogate markers of marker rs1058396 on Chromosome 18q12.3.
A

| Surrogate | Pos in NCBI | | Risk | Other | Seq |
|---|---|---|---|---|---|
| marker | Build 36 | R² | allele | allele | ID NO |
| rs7233769 | 41560679 | 0.923 | A | G | 1 |
| rs3819177 | 41570108 | 0.953 | T | C | 2 |
| rs692899 | 41570268 | 0.537 | C | T | 3 |
| rs1058396 | 41573517 | 1 | G | A | 4 |
| rs11660575 | 41574436 | 0.829 | A | G | 5 |
| rs7234986 | 41574602 | 0.874 | A | G | 6 |
| rs564409 | 41574712 | 0.461 | C | G | 7 |
| rs565153 | 41574769 | 0.497 | T | G | 8 |
| rs11082468 | 41575026 | 0.873 | A | G | 9 |
| rs11082469 | 41575267 | 0.879 | A | G | 10 |
| rs8086499 | 41575410 | 0.859 | A | G | 11 |
| rs12454680 | 41575415 | 0.81 | T | C | 12 |
| rs8086631 | 41575440 | 0.894 | C | T | 13 |
| rs56044725 | 41575561 | 0.895 | A | G | 14 |
| rs8087320 | 41575759 | 0.932 | G | C | 15 |
| rs12962485 | 41576408 | 0.44 | T | C | 16 |
| rs28897968 | 41576485 | 0.606 | G | C | 17 |
| rs493262 | 41577622 | 0.475 | G | A | 18 |
| rs6507640 | 41578062 | 0.663 | A | G | 19 |
| rs1682392 | 41579157 | 0.472 | G | A | 20 |
| rs4890588 | 41581292 | 0.469 | C | T | 21 |
| rs474270 | 41582293 | 0.464 | A | G | 22 |
| rs2282616 | 41582632 | 0.486 | G | A | 23 |
| rs2282615 | 41582748 | 0.463 | G | C | 24 |
| rs6507641 | 41583196 | 0.515 | A | G | 25 |
| rs17142 | 41583308 | 0.478 | A | G | 26 |
| rs3745006 | 41583588 | 0.687 | T | G | 27 |
| rs9954521 | 41584894 | 0.604 | T | A | 28 |
| rs1135980 | 41585281 | 0.433 | C | T | 29 |
| rs28903070* | 41585749 | 0.436 | T | A | 30 |
| rs3087560 | 41586253 | 0.63 | C | T | 31 |
| rs3178156 | 41586478 | 0.635 | A | C | 32 |
| rs11662680 | 41587183 | 0.585 | A | G | 33 |
| rs7359740 | 41587911 | 0.63 | G | A | 34 |
| rs1944336 | 41588324 | 0.477 | G | T | 35 |
| rs8090136 | 41588762 | 0.497 | C | T | 36 |
| rs8090390 | 41588774 | 0.494 | G | T | 37 |
| rs8090267 | 41588803 | 0.477 | C | T | 38 |
| rs11874337 | 41589256 | 0.395 | T | A | 39 |
| rs9953451 | 41589691 | 0.445 | T | C | 40 |
| rs9966818 | 41589897 | 0.458 | C | T | 41 |
| rs534637 | 41590141 | 0.489 | A | G | 42 |
| rs900970 | 41590556 | 0.442 | T | C | 43 |
| rs9947769 | 41591565 | 0.519 | G | T | 44 |
| rs4890300 | 41591993 | 0.459 | G | A | 45 |
| rs9951207 | 41592624 | 0.511 | G | A | 46 |
| rs9963415 | 41592825 | 0.502 | T | C | 47 |
| rs8096228 | 41593025 | 0.523 | C | T | 48 |
| rs59932916 | 41593069 | 0.429 | C | A | 49 |
| rs8096392 | 41593219 | 0.447 | A | G | 50 |
| rs572858 | 41594516 | 0.523 | G | A | 51 |
| rs2005378 | 41595420 | 0.569 | G | C | 52 |
| rs550201 | 41595770 | 0.526 | A | C | 53 |
| rs576687 | 41595792 | 0.569 | C | G | 54 |
| rs10502870 | 41596131 | 0.569 | A | G | 55 |
| rs3018180 | 41596964 | 0.268 | T | C | 56 |
| rs1625960 | 41597012 | 0.526 | G | A | 57 |
| rs1625985 | 41597016 | 0.532 | T | C | 58 |
| rs1626743 | 41597080 | 0.491 | A | G | 59 |
| rs1789558 | 41597184 | 0.538 | A | G | 60 |
| rs475584 | 41597415 | 0.559 | C | G | 61 |
| rs517221 | 41597628 | 0.557 | A | G | 62 |
| rs502339 | 41597987 | 0.523 | A | G | 63 |
| rs505060 | 41598280 | 0.545 | T | C | 64 |
| rs9959600 | 41600337 | 0.206 | G | T | 65 |
| rs9960093 | 41600802 | 0.212 | G | T | 66 |
| rs9959923 | 41600815 | 0.234 | C | A | 67 |
| rs2187408 | 41600839 | 0.208 | C | T | 68 |
| rs7237369 | 41601336 | 0.203 | C | T | 69 |
| rs7237823 | 41601433 | 0.217 | G | A | 70 |
| rs1115074 | 41612293 | 0.257 | A | G | 71 |
| rs1944333 | 41613122 | 0.232 | A | G | 72 |
| rs7506509 | 41613925 | 0.238 | T | C | 73 |
| rs1789553 | 41614138 | 0.237 | C | T | 74 |
| rs2187405 | 41617419 | 0.236 | C | G | 75 |
| rs539249 | 41618120 | 0.247 | A | C | 76 |
| rs4890301 | 41622267 | 0.25 | C | T | 77 |
| rs7241939 | 41623325 | 0.294 | G | A | 78 |
| rs7241734 | 41623487 | 0.275 | A | G | 79 |
| rs8083889 | 41623910 | 0.284 | G | T | 80 |
| rs4890592 | 41624961 | 0.298 | G | A | 81 |
| rs495078 | 41663873 | 0.273 | T | C | 82 |
| rs7230514 | 41626329 | 0.228 | T | A | 83 |
| rs2156610 | 41626469 | 0.237 | A | G | 84 |
| rs1944340 | 41626853 | 0.266 | G | C | 85 |
| rs4362470 | 41627762 | 0.282 | G | A | 86 |
| rs9953356 | 41628618 | 0.209 | G | T | 87 |
| rs9955590 | 41628958 | 0.264 | A | T | 88 |
| rs9956040 | 41629414 | 0.215 | A | G | 89 |
| rs11082474 | 41630283 | 0.277 | T | A | 90 |
| rs11082475 | 41630288 | 0.274 | G | A | 91 |
| rs7233627 | 41630717 | 0.252 | T | C | 92 |
| rs12457954 | 41631576 | 0.222 | A | G | 93 |
| rs12457989 | 41631771 | 0.282 | A | C | 94 |
| rs8084937 | 41633341 | 0.299 | A | G | 95 |
| rs8085076 | 41633410 | 0.271 | A | G | 96 |
| rs4890593 | 41634005 | 0.281 | C | T | 97 |
| rs559774 | 41634834 | 0.261 | A | G | 98 |
| rs4890594 | 41635463 | 0.257 | C | G | 99 |
| rs4890302 | 41635510 | 0.285 | G | A | 100 |
| rs7237600 | 41636812 | 0.267 | T | G | 101 |
| rs8095840 | 41637106 | 0.261 | G | A | 102 |
| rs6507646 | 41637703 | 0.254 | G | A | 103 |
| rs4890596 | 41638223 | 0.237 | C | T | 104 |
| rs1944339 | 41639040 | 0.292 | C | A | 105 |
| rs1944338 | 41639170 | 0.264 | G | A | 106 |
| rs484914 | 41639267 | 0.235 | T | C | 107 |
| rs563386 | 41640930 | 0.246 | C | G | 108 |
| rs9966999 | 41641160 | 0.247 | T | C | 109 |
| rs536784 | 41641545 | 0.276 | A | C | 110 |
| rs576309 | 41642213 | 0.244 | G | A | 111 |
| rs546739 | 41643152 | 0.207 | T | C | 112 |
| rs545070 | 41643303 | 0.232 | T | A | 113 |
| rs553007 | 41643483 | 0.254 | C | T | 114 |
| rs521133 | 41643641 | 0.231 | A | G | 115 |
| rs515762 | 41644205 | 0.241 | A | T | 116 |
| rs693488 | 41644637 | 0.225 | A | G | 117 |
| rs1789557 | 41644754 | 0.209 | A | C | 118 |
| rs1789556 | 41645163 | 0.282 | A | C | 119 |
| rs9966400 | 41646511 | 0.257 | T | A | 120 |
| rs9748917 | 41646575 | 0.225 | T | C | 121 |
| rs532613 | 41647517 | 0.27 | A | G | 122 |
| rs504030 | 41648373 | 0.226 | A | G | 123 |
| rs573463 | 41648521 | 0.212 | T | C | 124 |
| rs498545 | 41649711 | 0.245 | A | T | 125 |
| rs538405 | 41653433 | 0.238 | A | C | 126 |
| rs503331 | 41654092 | 0.241 | A | G | 127 |
| rs489653 | 41658448 | 0.218 | C | T | 128 |
| rs495078 | 41663873 | 0.279 | T | C | 129 |

B

| | Build36 | | Risk | Other | Seq |
|---|---|---|---|---|---|
| Marker | Postion | R² | allele | allele | ID NO |
| rs10460033 | 41557200 | 0.229969 | A | T | 135 |
| rs10460072 | 41557208 | 0.225427 | G | A | 136 |
| rs12963143 | 41557685 | 0.32201 | A | C | 137 |
| rs16978469 | 41559783 | 0.923926 | G | A | 138 |
| rs7234310 | 41560791 | 0.920508 | G | A | 139 |
| rs10432193 | 41561070 | 0.923207 | T | C | 140 |
| rs11877062 | 41561244 | 0.921977 | C | T | 130 |
| rs11877086 | 41561336 | 0.918935 | C | A | 141 |
| rs9304321 | 41562186 | 0.921852 | C | T | 142 |
| rs9304322 | 41562196 | 0.921757 | G | T | 143 |
| rs9304323 | 41562313 | 0.923237 | A | G | 144 |
| rs2170974 | 41562808 | 0.923788 | A | T | 145 |
| rs9967412 | 41562887 | 0.924013 | C | G | 146 |
| rs4479340 | 41562953 | 0.923288 | C | T | 147 |
| rs4316845 | 41563133 | 0.923826 | G | T | 148 |
| rs4310958 | 41563353 | 0.922973 | C | G | 149 |
| rs8087241 | 41563701 | 0.921248 | G | A | 150 |
| rs8088163 | 41563807 | 0.921677 | T | C | 151 |
| rs17674580 | 41563909 | 0.489916 | T | C | 152 |
| rs8090908 | 41564185 | 0.923912 | A | G | 153 |
| rs3819178 | 41564843 | 0.922224 | G | C | 154 |
| rs12963324 | 41564849 | 0.488927 | A | G | 155 |
| rs17674709 | 41565378 | 0.92356 | C | G | 156 |
| rs10460034 | 41565397 | 0.923926 | C | T | 157 |
| rs10460036 | 41565564 | 0.923423 | A | G | 158 |
| rs8099449 | 41565694 | 0.921686 | T | C | 159 |
| rs7229967 | 41566366 | 0.923509 | G | A | 160 |
| rs7229753 | 41566458 | 0.923926 | A | G | 161 |
| rs7230298 | 41566517 | 0.924184 | G | A | 162 |
| rs9946832 | 41566788 | 0.922155 | A | G | 163 |
| rs9946998 | 41566820 | 0.923729 | C | T | 164 |
| rs12455090 | 41567365 | 0.924213 | C | T | 165 |
| rs8096571 | 41568471 | 0.923711 | T | A | 166 |
| rs8095657 | 41568543 | 0.922352 | C | T | 167 |
| rs8083653 | 41569025 | 0.922692 | T | C | 168 |
| rs28898869 | 41569589 | 0.961109 | A | C | 169 |
| rs2298718 | 41570536 | 0.813038 | A | G | 170 |
| rs7238033 | 41570964 | 0.817394 | T | C | 171 |
| rs493363 | 41571247 | 0.531684 | A | G | 172 |
| rs10775480 | 41571280 | 0.819409 | T | C | 173 |
| rs11082466 | 41571526 | 0.817711 | C | T | 174 |
| rs10853535 | 41571545 | 0.817664 | C | T | 175 |
| rs11877028 | 41571864 | 0.999609 | C | G | 176 |
| rs11877630 | 41571975 | 0.999789 | A | G | 177 |
| rs17675121 | 41572183 | 0.997932 | T | C | 178 |
| rs11877720 | 41572253 | 0.999008 | A | G | 179 |
| rs11082467 | 41572642 | 0.99972 | T | C | 180 |
| rs9955503 | 41572692 | 0.660708 | A | T | 181 |
| rs11665385 | 41572744 | 0.999865 | G | A | 182 |
| rs473429 | 41573058 | 0.658081 | C | T | 183 |
| rs55723051 | 41585321 | 0.229219 | C | A | 184 |
| rs11082471 | 41599614 | 0.23525 | C | T | 185 |
| rs11082472 | 41599703 | 0.307919 | T | A | 186 |
| rs35702919 | 41599705 | 0.331755 | T | A | 187 |
| rs2156611 | 41599862 | 0.235398 | C | T | 188 |
| rs2156612 | 41599877 | 0.23274 | C | T | 189 |
| rs2187406 | 41600017 | 0.233978 | G | T | 190 |
| rs2187407 | 41600038 | 0.234041 | C | T | 191 |
| rs12457207 | 41600113 | 0.234179 | G | C | 192 |
| rs9959453 | 41600347 | 0.230845 | C | T | 193 |
| rs9948723 | 41600462 | 0.2353 | T | G | 194 |
| rs9959480 | 41600467 | 0.235028 | A | G | 195 |
| rs9948733 | 41600486 | 0.235677 | T | C | 196 |
| rs2187409 | 41600896 | 0.231358 | T | C | 197 |
| rs7237218 | 41601280 | 0.225405 | C | T | 198 |
| rs7237722 | 41630942 | 0.278823 | T | C | 199 |
| rs7236814 | 41630943 | 0.268958 | G | A | 200 |
| rs7236744 | 41631138 | 0.30923 | A | C | 201 |
| rs7237029 | 41631229 | 0.311759 | C | G | 202 |
| rs6507645 | 41635799 | 0.363613 | T | C | 203 |
| rs9954025 | 41646268 | 0.320274 | G | A | 204 |
| rs1440822 | 41659790 | 0.216897 | T | C | 205 |
| rs515373 | 41660929 | 0.214746 | G | A | 206 |
| rs12326162 | 41663680 | 0.234552 | C | T | 207 |
| rs4890303 | 41675437 | 0.205304 | C | T | 208 |

Markers were identified based on sequence data from an Icelandic dataset comprising whole-genome sequences of approximately 300 individuals (A) and from an additional 800 individuals (B).
Shown are the marker names, their location in NCBI build 36, numerical values of the linkage disequilibrium measure r² to rs1058396, the risk alleles for the surrogate markers, i.e. alleles that are correlated with the at-risk G allele of the anchor marker rs1058396, and the other allele, and lastly a sequence listing number, identifying the flanking sequence of each particular surrogate marker.
*Also known as rs544373

Measures of susceptibility or risk include measures such as relative risk (RR), odds ratio (OR), and absolute risk (AR), as described in more detail herein.

In certain embodiments, increased susceptibility refers to a risk with values of RR or OR of at least 1.10, at least 1.11, at least 1.12, at least 1.13, at least 1.14, at least 1.15, at least 1.16, at least 1.17, at least 1.18, at least 1.19, at least 1.20, at least 1.21, at least 1.22, at least 1.23, at least 1.24, at least 1.25, at least 1.30, at least 1.35, at least 1.40, at least 1.45, at least 1.50, at least 1.55, at least 1.60, at least 1.65, at least 1.70, at least 1.75, and/or at least 1.80. Other numerical non-integer values greater than unity are also possible to characterize the risk, and such numerical values are also within scope of the invention. Certain embodiments relate to homozygous individuals for a particular marker, i.e. individuals who carry two copies of the same allele in their genome. One preferred embodiment relates to individuals who are homozygous carriers of the A allele of rs1058396, or a marker allele in linkage disequilibrium therewith.

In certain other embodiments, determination of the presence of particular marker alleles or particular haplotypes is predictive of a decreased susceptibility of urinary bladder cancer in humans. For SNP markers with two alleles, the alternate allele to an at-risk allele will be in decreased frequency in patients compared with controls. Thus, determination of the presence of the alternate allele is indicative of a decreased susceptibility of urinary bladder cancer. Individuals who are homozygous for the alternate (protective) allele are at particularly decreased susceptibility or risk.

To identify markers that are useful for assessing susceptibility to urinary bladder cancer, it may be useful to compare the frequency of markers alleles in individuals with urinary bladder cancer to control individuals. The control individuals may be a random sample from the general population, i.e. a population cohort. The control individuals may also be a sample from individuals that are disease-free, e.g. individuals who have been confirmed not to have urinary bladder cancer. In one embodiment, an increase in frequency of at least one allele in at least one polymorphism in individuals diagnosed with urinary bladder cancer, as compared with the frequency of the at least one allele in the control group is indicative of the at least one allele being useful for assessing increased susceptibility to urinary bladder cancer. In another embodiment, a decrease in frequency of at least one allele in at least one polymorphism in individuals diagnosed with urinary bladder cancer, as compared with the frequency of the at least one allele in the control sample is indicative of the at least one allele being useful for assessing decreased susceptibility to, or protection against, urinary bladder cancer.

In general, sequence data can be obtained by analyzing a sample from an individual, or by analyzing information about specific markers in a database or other data collection, for example a genotype database or a sequence database. The sample is in certain embodiments a nucleic acid sample, or a sample that contains nucleic acid material. Analyzing a sample from an individual may in certain embodiments include steps of isolating genomic nucleic acid from the sample, amplifying a segment of the genomic nucleic acid that contains at least one polymorphic marker, and determine sequence information about the at least one polymorphic marker. Amplification is preferably performed by Polymerase Chain Reaction (PCR) techniques. In certain embodiments, sequence data can be obtained through nucleic acid sequence information or amino acid sequence information from a preexisting record. Such a preexisting record can be any documentation, database or other form of data storage containing such information.

Determination of a susceptibility or risk of a particular individual in general comprises comparison of the genotype information (sequence information) to a record or database providing a correlation about particular polymorphic marker(s) and susceptibility to disease, such as urinary bladder cancer. Thus, in specific embodiments, determining a susceptibility comprises comparing the sequence data to a database containing correlation data between the at least one polymorphic marker and susceptibility to urinary bladder cancer. In certain embodiments, the database comprises at least one measure of susceptibility to urinary bladder cancer for the at least one polymorphic marker. In certain embodiments, the database comprises a look-up table comprising at least one measure of susceptibility to urinary bladder cancer for the at least one polymorphic marker. The measure of susceptibility may in the form of relative risk (RR), absolute risk (AR), percentage (%) or other convenient measure for describing genetic susceptibility of individuals.

In certain embodiments of the invention, more than one polymorphic marker is analyzed. In certain embodiments, at least two polymorphic markers are analyzed. Thus, in certain embodiments, sequence data about at least two polymorphic markers is obtained.

In certain embodiments, a further step of analyzing at least one haplotype comprising two or more polymorphic markers is included. Any convenient method for haplotype analysis known to the skilled person may be employed in such embodiments.

One aspect of the invention relates to a method for determining a susceptibility to urinary bladder cancer in a human individual, comprising determining the presence or absence of at least one allele of at least one polymorphic marker in a nucleic acid sample obtained from the individual, or in a genotype dataset from the individual, wherein the at least one polymorphic marker is selected from the group consisting of rs1058396, and markers in linkage disequilibrium therewith, and wherein determination of the presence of the at least one allele is indicative of a susceptibility to urinary bladder cancer. Determination of the presence of an allele that correlates with urinary bladder cancer is indicative of an increased susceptibility to urinary bladder cancer. Individuals who are homozygous for such alleles are particularly susceptible (i.e., at particularly high risk) to urinary bladder cancer. On the other hand, individuals who do not carry such at-risk alleles are at a decreased susceptibility of developing urinary bladder cancer, as compared with a random sample from the population. For SNPs, such individuals will be homozygous for the alternate (protective) allele of the polymorphism.

Determination of susceptibility is in some embodiments reported by a comparison with non-carriers of the at-risk allele(s) of polymorphic markers. In certain embodiments, susceptibility is reported based on a comparison with the general population, e.g. compared with a random selection of individuals from the population.

Another aspect of the methods of the invention relates to a method of determining susceptibility to bladder cancer, the method comprising analyzing nucleic acid sequence data representative of at least one allele of the SLC14A1 gene in a human subject, wherein different alleles of the SLC14A1 gene are associated with different susceptibilities to bladder cancer in humans, and determining a susceptibility to bladder cancer for the human subject from the data. In certain embodiments, the analyzing nucleic acid sequence data comprises analyzing a biological sample from the human subject to obtain information selected from the group consisting of (a) nucleic acid sequence information, wherein the nucleic acid sequence information comprises sequence sufficient to identify the presence or absence of at least one allele of the SLC14A1 gene in the subject; (b) nucleic acid sequence information, wherein the nucleic acid sequence information identifies at least one allele of a polymorphic marker in linkage disequilibrium (LD) with an SLC14A1 allele associated with bladder cancer in humans, wherein the LD is characterized by a value for r² of at least 0.2; (c) measurement of the quantity or length of SLC14A1 mRNA, wherein the measurement is indicative of the presence or absence of the allele; (d) measurement of the quantity of SLC14A1 protein, wherein the measurement is indicative of the presence or absence of the allele; and (e) measurement of SLC14A1 activity, wherein the measurement is indicative of the presence or absence of the allele. The SLC14A1 activity may for example be urea transport or urea binding activity. The SLC14A1 activity may also be identity of the Kidd blood group status of an individual expressing SLC14A1 protein containing the allele. In certain embodiments, the method further comprises obtaining a biological sample comprising nucleic acid from the human subject. In certain embodiments, the analyzing data may comprise analyzing data from a preexisting record about the human subject.

### Obtaining Nucleic Acid Sequence Data

Sequence data can be nucleic acid sequence data or protein sequence data, which may be obtained by means known in the art. Nucleic acid sequence data is suitably obtained from a biological sample of genomic DNA, RNA, or cDNA (a “test sample”) from an individual (“test subject). For example, nucleic acid sequence data may be obtained through direct analysis of the sequence of the polymorphic position (allele) of a polymorphic marker. Suitable methods, some of which are described herein, include, for instance, whole genome sequencing methods, whole genome analysis using SNP chips (e.g., Infinium HD BeadChip), cloning for polymorphisms, non-radioactive PCR-single strand conformation polymorphism analysis, denaturing high pressure liquid chromatography (DHPLC), DNA hybridization, computational analysis, single-stranded conformational polymorphism (SSCP), restriction fragment length polymorphism (RFLP), automated fluorescent sequencing; clamped denaturing gel electrophoresis (CDGE); denaturing gradient gel electrophoresis (DGGE), mobility shift analysis, restriction enzyme analysis; heteroduplex analysis, chemical mismatch cleavage (CMC), RNase protection assays, use of polypeptides that recognize nucleotide mismatches, such as E. coli mutS protein, allele-specific PCR, and direct manual and automated sequencing. These and other methods are described in the art (see, for instance, Li et al. , Nucleic Acids Research, 28(2): e1 (i-v) (2000); Liu et al. , Biochem Cell Bio 80:17-22 (2000); and Burczak et al. , Polymorphism Detection and Analysis, Eaton Publishing, 2000; Sheffield et al. , Proc. Natl. Acad. Sci. USA, 86:232-236 (1989); Orita et al. , Proc. Natl. Acad. Sci. USA, 86:2766-2770 (1989); Flavell et al. , Cell, 15:25-41 (1978); Geever et al. , Proc. Natl. Acad. Sci. USA, 78:5081-5085 (1981); Cotton et al. , Proc. Natl. Acad. Sci. USA, 85:4397-4401 (1985); Myers et al. , Science 230:1242-1246 (1985); Church and Gilbert, Proc. Natl. Acad. Sci. USA, 81:1991-1995 (1988); Sanger et al. , Proc. Natl. Acad. Sci. USA, 74:5463-5467 (1977); and Beavis et al., U.S. Pat. No. 5,288,644).

Recent technological advances have resulted in technologies that allow massive parallel sequencing to be performed in relatively condensed format. These technologies share sequencing-by-synthesis principle for generating sequence information, with different technological solutions implemented for extending, tagging and detecting sequences. Exemplary technologies include 454 pyrosequencing technology (Nyren, P. et al. Anal Biochem 208:171-75 (1993); http://www.454.com), Illumina Solexa sequencing technology (Bentley, D. R. Curr Opin Genet Dev 16:545-52 (2006); http://www.illumina.com), and the SOLID technology developed by Applied Biosystems (ABI) (http://www.appliedbiosystems.com; see also Strausberg, R. L., et al. Drug Disc Today 13:569-77 (2008)). Other sequencing technologies include those developed by Pacific Biosciences (http://www.pacificbiosciences.com), Complete Genomics (http://www.completegenomics.com), Intelligen Bio-Systems (http://www.intelligentbiosystems.com), Genome Corp (http://www.genomecorp.com), ION Torrent Systems (http://www.iontorrent.com) and Helicos Biosciences (http://www.helicosbio.som). It is contemplated that sequence data useful for performing the present invention may be obtained by any such sequencing method, or other sequencing methods that are developed or made available. Thus, any sequence method that provides the allelic identity at particular polymorphic sites (e.g., the absence or presence of particular alleles at particular polymorphic sites) is useful in the methods described and claimed herein.

Alternatively, hybridization methods may be used (see Current Protocols in Molecular Biology, Ausubel et al., eds., John Wiley & Sons, including all supplements). For example, a biological sample of genomic DNA, RNA, or cDNA (a “test sample”) may be obtained from a test subject. The subject can be an adult, child, or fetus. The DNA, RNA, or cDNA sample is then examined. The presence of a specific marker allele can be indicated by sequence-specific hybridization of a nucleic acid probe specific for the particular allele. The presence of more than one specific marker allele or a specific haplotype can be indicated by using several sequence-specific nucleic acid probes, each being specific for a particular allele. A sequence-specific probe can be directed to hybridize to genomic DNA, RNA, or cDNA. A “nucleic acid probe”, as used herein, can be a DNA probe or an RNA probe that hybridizes to a complementary sequence. One of skill in the art would know how to design such a probe so that sequence specific hybridization will occur only if a particular allele is present in a genomic sequence from a test sample.

To diagnose a susceptibility to Bladder Cancer, a hybridization sample can be formed by contacting the test sample, such as a genomic DNA sample, with at least one nucleic acid probe. A non-limiting example of a probe for detecting mRNA or genomic DNA is a labeled nucleic acid probe that is capable of hybridizing to mRNA or genomic DNA sequences described herein. The nucleic acid probe can be, for example, a full-length nucleic acid molecule, or a portion thereof, such as an oligonucleotide of at least 10, 15, 30, 50, 100, 250 or 500 nucleotides in length that is sufficient to specifically hybridize under stringent conditions to appropriate mRNA or genomic DNA. For example, the nucleic acid probe can comprise all or a portion of the nucleotide sequence of the SLC14A1 gene, or the probe can be the complementary sequence of such a sequence. Hybridization can be performed by methods well known to the person skilled in the art (see, e.g., Current Protocols in Molecular Biology, Ausubel et al., eds., John Wiley & Sons, including all supplements). In one embodiment, hybridization refers to specific hybridization, i.e., hybridization with no mismatches (exact hybridization). In one embodiment, the hybridization conditions for specific hybridization are high stringency.

Specific hybridization, if present, is detected using standard methods. If specific hybridization occurs between the nucleic acid probe and the nucleic acid in the test sample, then the sample contains the allele that is complementary to the nucleotide that is present in the nucleic acid probe.

Additionally, or alternatively, a peptide nucleic acid (PNA) probe can be used in addition to, or instead of, a nucleic acid probe in the hybridization methods described herein. A PNA is a DNA mimic having a peptide-like, inorganic backbone, such as N-(2-aminoethyl)glycine units, with an organic base (A, G, C, T or U) attached to the glycine nitrogen via a methylene carbonyl linker (see, for example, Nielsen et al. , Bioconjug. Chem. 5:3-7 (1994)). The PNA probe can be designed to specifically hybridize to a molecule in a sample suspected of containing one or more of the marker alleles or haplotypes that are associated with eosinophilia, asthma, myocardial infarction, and/or hypertension.

In one embodiment of the invention, a test sample containing genomic DNA obtained from the subject is collected and the polymerase chain reaction (PCR) is used to amplify a fragment comprising one or more polymorphic marker. As described herein, identification of particular marker alleles can be accomplished using a variety of methods. In another embodiment, determination of a susceptibility is accomplished by expression analysis, for example using quantitative PCR (kinetic thermal cycling). This technique can, for example, utilize commercially available technologies, such as TaqMan® (Applied Biosystems, Foster City, Calif.). The technique can for example assess the presence of an alteration in the expression or composition of a polypeptide or splicing variant(s) that is encoded by a nucleic acid associated described herein. Alternatively, this technique may assess expression levels of genes or particular splice variants of genes, that are affected by one or more of the variants described herein. Further, the expression of the variant(s) can be quantified as physically or functionally different.

Allele-specific oligonucleotides can also be used to detect the presence of a particular allele in a nucleic acid. An “allele-specific oligonucleotide” (also referred to herein as an “allele-specific oligonucleotide probe”) is an oligonucleotide of any suitable size, for example an oligonucleotide of approximately 10-50 base pairs or approximately 15-30 base pairs, that specifically hybridizes to a nucleic acid which contains a specific allele at a polymorphic site (e.g., a polymorphic marker). An allele-specific oligonucleotide probe that is specific for one or more particular alleles at polymorphic markers can be prepared using standard methods (see, e.g., Current Protocols in Molecular Biology, supra). PCR can be used to amplify the desired region. Specific hybridization of an allele-specific oligonucleotide probe to DNA from a subject is indicative of the presence of a specific allele at a polymorphic site (see, e.g., Gibbs et al. , Nucleic Acids Res. 17:2437-2448 (1989) and WO 93/22456).

With the addition of analogs such as locked nucleic acids (LNAs), the size of primers and probes can be reduced to as few as 8 bases. LNAs are a novel class of bicyclic DNA analogs in which the 2′ and 4′ positions in the furanose ring are joined via an O-methylene (oxy-LNA), S-methylene (thio-LNA), or amino methylene (amino-LNA) moiety. Common to all of these LNA variants is an affinity toward complementary nucleic acids, which is by far the highest reported for a DNA analog. For example, particular all oxy-LNA nonamers have been shown to have melting temperatures (Tm) of 64° C. and 74° C. when in complex with complementary DNA or RNA, respectively, as opposed to 28° C. for both DNA and RNA for the corresponding DNA nonamer. Substantial increases in Tm are also obtained when LNA monomers are used in combination with standard DNA or RNA monomers. For primers and probes, depending on where the LNA monomers are included (e.g., the 3′ end, the 5′ end, or in the middle), the Tm could be increased considerably. It is therefore contemplated that in certain embodiments, LNAs are used to detect particular alleles at polymorphic sites associated with particular vascular conditions, as described herein.

In certain embodiments, arrays of oligonucleotide probes that are complementary to target nucleic acid sequence segments from a subject, can be used to identify polymorphisms in a nucleic acid. For example, an oligonucleotide array can be used. Oligonucleotide arrays typically comprise a plurality of different oligonucleotide probes that are coupled to a surface of a substrate in different known locations. These arrays can generally be produced using mechanical synthesis methods or light directed synthesis methods that incorporate a combination of photolithographic methods and solid phase oligonucleotide synthesis methods, or by other methods known to the person skilled in the art (see, e.g., Bier et al. , Adv Biochem Eng Biotechnol 109:433-53 (2008); Hoheisel, Nat Rev Genet. 7:200-10 (2006); Fan et al. , Methods Enzymol 410:57-73 (2006); Raqoussis & Elvidge, Expert Rev Mol Diagn 6:145-52 (2006); Mockler et al. , Genomics 85:1-15 (2005), and references cited therein, the entire teachings of each of which are incorporated by reference herein). Many additional descriptions of the preparation and use of oligonucleotide arrays for detection of polymorphisms can be found, for example, in U.S. Pat. No. 6,858,394, U.S. Pat. No. 6,429,027, U.S. Pat. No. 5,445,934, U.S. Pat. No. 5,700,637, U.S. Pat. No. 5,744,305, U.S. Pat. No. 5,945,334, U.S. Pat. No. 6,054,270, U.S. Pat. No. 6,300,063, U.S. Pat. No. 6,733,977, U.S. Pat. No. 7,364,858, EP 619 321, and EP 373 203, the entire teachings of which are incorporated by reference herein.

Also, standard techniques for genotyping can be used to detect particular marker alleles, such as fluorescence-based techniques (e.g., Chen et al. , Genome Res. 9(5): 492-98 (1999); Kutyavin et al., Nucleic Acid Res. 34:e128 (2006)), utilizing PCR, LCR, Nested PCR and other techniques for nucleic acid amplification. Specific commercial methodologies available for SNP genotyping include, but are not limited to, TaqMan genotyping assays and SNPlex platforms (Applied Biosystems), gel electrophoresis (Applied Biosystems), mass spectrometry (e.g., MassARRAY system from Sequenom), minisequencing methods, real-time PCR, Bio-Plex system (BioRad), CEQ and SNPstream systems (Beckman), array hybridization technology (e.g., Affymetrix GeneChip; Perlegen), BeadArray Technologies (e.g., Illumina GoldenGate and Infinium assays), array tag technology (e.g., Parallele), and endonuclease-based fluorescence hybridization technology (Invader; Third Wave).

Suitable biological sample in the methods described herein can be any sample containing nucleic acid (e.g., genomic DNA) and/or protein from the human individual. For example, the biological sample can be a blood sample, a serum sample, a leukapheresis sample, an amniotic fluid sample, a cerbrospinal fluid sample, a hair sample, a tissue sample from skin, muscle, buccal, or conjuctival mucosa, placenta, gastrointestinal tract, or other organs, a semen sample, a urine sample, a saliva sample, a nail sample, a tooth sample, and the like. Preferably, the sample is a blood sample, a salive sample or a buccal swab.

### Protein Analysis

Missense nucleic acid variations may lead to an altered amino acid sequence, as compared to the non-variant (e.g., wild-type) protein, due to one or more amino acid substitutions, deletions, or insertions, or truncation (due to, e.g., splice variation). In such instances, detection of the amino acid substitution of the variant protein may be useful. This way, nucleic acid sequence data may be obtained through indirect analysis of the nucleic acid sequence of the allele of the polymorphic marker, i.e. by detecting a protein variation. Methods of detecting variant proteins are known in the art. For example, direct amino acid sequencing of the variant protein followed by comparison to a reference amino acid sequence can be used. Alternatively, SDS-PAGE followed by gel staining can be used to detect variant proteins of different molecular weights. Also, Immunoassays, e.g., immunofluorescent immunoassays, immunoprecipitations, radioimmunoasays, ELISA, and Western blotting, in which an antibody specific for an epitope comprising the variant sequence among the variant protein and non-variant or wild-type protein can be used. In certain embodiments of the present invention, the identity of amino acids at particular position in an encoded SLC14A1 protein is determined. In certain embodiments, identity of amino acids at one or more of positions 4, 44, 167 and 280 in a SLC14A1 protein with sequence as set forth in SEQ ID NO:133 is determined. In certain embodiments, determination of the presence or absence an amino acid selected from the group consisting of Tryptophan at position 4, Glutamic Acid at position 100, Valine at position 167 and Aspartic Acid at position 280 is indicative of risk of Bladder Cancer. In certain embodiments, determination of the presence of an amino acid selected from the group consisting of Tryptophan at position 4, Glutamic Acid at position 100, Valine at position 167 and Aspartic Acid at position 280 is indicative of increased risk of Bladder Cancer. The detection may be suitably performed using any of the methods described in the above.

Thus, one aspect of the invention relates to a method of determining a susceptibility to bladder cancer, the method comprising obtaining amino acid sequence data about at least one encoded SLC14A1 protein in a human individual; and analyzing the amino acid sequence data to determine whether at least one amino acid substitution predictive of increased susceptibility of bladder cancer is present, wherein a determination of the presence of the at least one amino acid substitution is indicative of increased susceptibility of bladder cancer for the individual, and wherein a determination of the absence of the at least one amino acid substitution is indicative of the individual not having the increased susceptibility.

In certain embodiments, the method further comprises obtaining a biological sample containing protein from the individual, and obtain amino acid sequence data about SLC14A1 protein from the sample. Sequence information about the SLC14A1 protein may suitably obtained using a method selected from protein sequencing and antibody assay methods.

In some cases, a variant protein has altered (e.g., upregulated or downregulated) biological activity, in comparison to the non-variant or wild-type protein. The biological activity can be, for example, a binding activity or enzymatic activity. In this instance, altered biological activity may be used to detect a variation in protein encoded by a nucleic acid sequence variation. Methods of detecting binding activity and enzymatic activity are known in the art and include, for instance, ELISA, competitive binding assays, quantitative binding assays using instruments such as, for example, a Biacore® 3000 instrument, chromatographic assays, e.g., HPLC and TLC.

Alternatively or additionally, a protein variation encoded by a genetic variation could lead to an altered expression level, e.g., an increased expression level of an mRNA or protein, a decreased expression level of an mRNA or protein. In such instances, nucleic acid sequence data about the allele of the polymorphic marker, or protein sequence data about the protein variation, can be obtained through detection of the altered expression level. Methods of detecting expression levels are known in the art. For example, ELISA, radioimmunoassays, immunofluorescence, and Western blotting can be used to compare the expression of protein levels. Alternatively, Northern blotting can be used to compare the levels of mRNA. These processes are described in Sambrook et al. , Molecular Cloning: A Laboratory Manual, 3^{rd} ed. Cold Spring Harbor Laboratory Press, Cold Spring Harbor, N.Y. (2001).

Any of these methods may be performed using a nucleic acid (e.g., DNA, mRNA) or protein of a biological sample obtained from the human individual for which a susceptibility is being determined. The biological sample can be any nucleic acid or protein containing sample obtained from the human individual. For example, the biological sample can be any of the biological samples described herein.

### Number of Polymorphic Markers/Genes Analyzed

With regard to the methods of determining a susceptibility described herein, the methods can comprise obtaining sequence data about any number of polymorphic markers and/or about any number of genes. For example, the method can comprise obtaining sequence data for about at least 1, 2, 3, 4, 5, 6, 7, 8, 9, 10, 15, 20, 25, 30, 40, 50, 100, 500, 1000, 10,000 or more polymorphic markers. In certain embodiments, the sequence data is obtained from a microarray comprising probes for detecting a plurality of markers. The markers can be independent of rs1058396, rs11877062, rs2298720 and/or rs2298719 and/or the markers may be in linkage disequilibrium with any one of rs1058396, rs11877062, rs2298720 and/or rs2298719. The polymorphic markers can be the ones of the group specified herein or they can be different polymorphic markers that are not listed herein. In a specific embodiment, the method comprises obtaining sequence data about at least two polymorphic markers. In certain embodiments, each of the markers may be associated with a different gene. For example, in some instances, if the method comprises obtaining nucleic acid data about a human individual identifying at least one allele of a polymorphic marker, then the method comprises identifying at least one allele of at least one polymorphic marker. Also, for example, the method can comprise obtaining sequence data about a human individual identifying alleles of multiple, independent markers, which are not in linkage disequilibrium.

### Linkage Disequilibrium

Linkage Disequilibrium (LD) refers to a non-random assortment of two genetic elements. For example, if a particular genetic element (e.g., an allele of a polymorphic marker, or a haplotype) occurs in a population at a frequency of 0.50 (50%) and another element occurs at a frequency of 0.50 (50%), then the predicted occurrence of a person's having both elements is 0.25 (25%), assuming a random distribution of the elements. However, if it is discovered that the two elements occur together at a frequency higher than 0.25, then the elements are said to be in linkage disequilibrium, since they tend to be inherited together at a higher rate than what their independent frequencies of occurrence (e.g., allele or haplotype frequencies) would predict. Roughly speaking, LD is generally correlated with the frequency of recombination events between the two elements. Allele or haplotype frequencies can be determined in a population by genotyping individuals in a population and determining the frequency of the occurrence of each allele or haplotype in the population. For populations of diploids, e.g., human populations, individuals will typically have two alleles for each genetic element (e.g., a marker, haplotype or gene).

Many different measures have been proposed for assessing the strength of linkage disequilibrium (LD; reviewed in Devlin, B. & Risch, N., Genomics 29:311-22 (1995)). Most capture the strength of association between pairs of biallelic sites. Two important pairwise measures of LD are r² (sometimes denoted Δ²) and |D′| (Lewontin, R., Genetics 49:49-67 (1964); Hill, W. G. & Robertson, A. Theor. Appl. Genet. 22:226-231 (1968)). Both measures range from 0 (no disequilibrium) to 1 (‘complete’ disequilibrium), but their interpretation is slightly different. |D′| is defined in such a way that it is equal to 1 if just two or three of the possible haplotypes are present, and it is <1 if all four possible haplotypes are present. Therefore, a value of |D′| that is <1 indicates that historical recombination may have occurred between two sites (recurrent mutation can also cause |D′| to be <1, but for single nucleotide polymorphisms (SNPs) this is usually regarded as being less likely than recombination). The measure r² represents the statistical correlation between two sites, and takes the value of 11f only two haplotypes are present.

The r² measure is arguably the most relevant measure for association mapping, because there is a simple inverse relationship between r² and the sample size required to detect association between susceptibility loci and SNPs. These measures are defined for pairs of sites, but for some applications a determination of how strong LD is across an entire region that contains many polymorphic sites might be desirable (e.g., testing whether the strength of LD differs significantly among loci or across populations, or whether there is more or less LD in a region than predicted under a particular model). Measuring LD across a region is not straightforward, but one approach is to use the measure r, which was developed in population genetics. Roughly speaking, r measures how much recombination would be required under a particular population model to generate the LD that is seen in the data. This type of method can potentially also provide a statistically rigorous approach to the problem of determining whether LD data provide evidence for the presence of recombination hotspots.

For the methods described herein, a significant r² value between sites can be at least 0.1 such as at least 0.1, 0.15, 0.2, 0.25, 0.3, 0.35, 0.4, 0.45, 0.5, 0.55, 0.6, 0.65, 0.7, 0.75, 0.8, 0.85, 0.9, 0.91, 0.92, 0.93, 0.94, 0.95, 0.96, 0.97, 0.98, 0.99 or 1.0. In one specific embodiment of invention, the significant r² value can be at least 0.2. In another specific embodiment of invention, the significant r² value can be at least 0.5. In one specific embodiment of invention, the significant r² value can be at least 0.8. Alternatively, linkage disequilibrium as described herein, refers to linkage disequilibrium characterized by values of r² of at least 0.2, such as 0.3, 0.4, 0.5, 0.6, 0.7, 0.8, 0.85, 0.9, 0.95, 0.96, 0.97, 0.98, 0.99. Thus, linkage disequilibrium represents a correlation between alleles of distinct markers. It is measured by correlation coefficient or |D′| (r² up to 1.0 and |D′| up to 1.0). Linkage disequilibrium can be determined in a single human population, as defined herein, or it can be determined in a collection of samples comprising individuals from more than one human population. In one embodiment of the invention, LD is determined in a sample from one or more of the HapMap populations. These include samples from the Yoruba people of Ibadan, Nigeria (YR1), samples from individuals from the Tokyo area in Japan (JPT), samples from individuals Beijing, China (CHB), and samples from U.S. residents with northern and western European ancestry (CEU), as described (The International HapMap Consortium, Nature 426:789-796 (2003)). In one such embodiment, LD is determined in the Caucasian CEU population of the HapMap samples. In another embodiment, LD is determined in the African YR1 population. In yet another embodiment, LD is determined in samples from the Icelandic population.

If all polymorphisms in the genome were independent at the population level (i.e., no LD between polymorphisms), then every single one of them would need to be investigated in association studies, to assess all different polymorphic states. However, due to linkage disequilibrium between polymorphisms, tightly linked polymorphisms are strongly correlated, which reduces the number of polymorphisms that need to be investigated in an association study to observe a significant association. Another consequence of LD is that many polymorphisms may give an association signal due to the fact that these polymorphisms are strongly correlated.

Genomic LD maps have been generated across the genome, and such LD maps have been proposed to serve as framework for mapping disease-genes (Risch, N. & Merkiangas, K, Science 273: 1516-1517 (1996); Maniatis, N., et al. , Proc Natl Acad Sci USA 99:2228-2233 (2002); Reich, D E et al, Nature 411:199-204 (2001)).

It is now established that many portions of the human genome can be broken into series of discrete haplotype blocks containing a few common haplotypes; for these blocks, linkage disequilibrium data provides little evidence indicating recombination (see, e.g., Wall., J. D. and Pritchard, J. K., Nature Reviews Genetics 4:587-597 (2003); Daly, M. et al. , Nature Genet. 29:229-232 (2001); Gabriel, S. B. et al. , Science 296:2225-2229 (2002); Patil, N. et al. , Science 294:1719-1723 (2001); Dawson, E. et al. , Nature 418:544-548 (2002); Phillips, M. S. et al. , Nature Genet. 33:382-387 (2003)).

Haplotype blocks (LD blocks) can be used to map associations between phenotype and haplotype status, using single markers or haplotypes comprising a plurality of markers. The main haplotypes can be identified in each haplotype block, and then a set of “tagging” SNPs or markers (the smallest set of SNPs or markers needed to distinguish among the haplotypes) can then be identified. These tagging SNPs or markers can then be used in assessment of samples from groups of individuals, in order to identify association between phenotype and haplotype. If desired, neighboring haplotype blocks can be assessed concurrently, as there may also exist linkage disequilibrium among the haplotype blocks.

It has thus become apparent that for any given observed association to a polymorphic marker in the genome, it is likely that additional markers in the genome also show association. This is a natural consequence of the uneven distribution of LD across the genome, as observed by the large variation in recombination rates. The markers used to detect association thus in a sense represent “tags” for a genomic region (i.e., a haplotype block or LD block) that is associating with a given disease or trait, and as such are useful for use in the methods and kits of the invention.

By way of example, the markers rs1058396, rs11877062, rs2298720 and rs2298719 may be detected directly to determine risk of Bladder Cancer. Alternatively, any marker in linkage disequilibrium with these markers may be detected to determine risk.

The present invention thus refers to the rs1058396, rs11877062, rs2298720 and rs2298719 markers used for detecting association to Bladder Cancer, as well as markers in linkage disequilibrium with these markers. Thus, in certain embodiments of the invention, markers that are in LD with these markers and/or haplotypes of the invention, as described herein, may be used as surrogate markers.

Suitable surrogate markers may be selected using public information, such as from the International HapMap Consortium (http://www.hapmap.org) and the International 1000genomes Consortium (http://www.1000genomes.org). The stronger the linkage disequilibrium to the anchor marker, the better the surrogate, and thus the mores similar the association detected by the surrogate is expected to be to the association detected by the anchor marker. Markers with values of r² equal to 1 are perfect surrogates for the at-risk variants, i.e. genotypes for one marker perfectly predicts genotypes for the other. In other words, the surrogate will, by necessity, give exactly the same association data to any particular disease as the anchor marker. Markers with smaller values of r² than 1 can also be surrogates for the at-risk anchor variant.

The present invention encompasses the assessment of such surrogate markers for the markers as disclosed herein. Such markers are annotated, mapped and listed in public databases, as well known to the skilled person, or can alternatively be readily identified by sequencing the region or a part of the region identified by the markers of the present invention in a group of individuals, and identify polymorphisms in the resulting group of sequences. As a consequence, the person skilled in the art can readily and without undue experimentation identify and select appropriate surrogate markers.

### Association Analysis

For single marker association to a disease, the Fisher exact test can be used to calculate two-sided p-values for each individual allele. Correcting for relatedness among patients can be done by extending a variance adjustment procedure previously described (Risch, N. & Teng, J. Genome Res., 8:1273-1288 (1998)) for sibships so that it can be applied to general familial relationships. The method of genomic controls (Devlin, B. & Roeder, K. Biometrics 55:997 (1999)) can also be used to adjust for the relatedness of the individuals and possible stratification.

For both single-marker and haplotype analyses, relative risk (RR) and the population attributable risk (PAR) can be calculated assuming a multiplicative model (haplotype relative risk model) (Terwilliger, J. D. & Ott, J., Hum. Hered. 42:337-46 (1992) and Falk, C. T. & Rubinstein, P, Ann. Hum. Genet. 51 (Pt 3):227-33 (1987)), i.e., that the risks of the two alleles/haplotypes a person carries multiply. For example, if RR is the risk of A relative to a, then the risk of a person homozygote AA will be RR times that of a heterozygote Aa and RR² times that of a homozygote aa. The multiplicative model has a nice property that simplifies analysis and computations—haplotypes are independent, i.e., in Hardy-Weinberg equilibrium, within the affected population as well as within the control population. As a consequence, haplotype counts of the affecteds and controls each have multinomial distributions, but with different haplotype frequencies under the alternative hypothesis. Specifically, for two haplotypes, hᵢ and hⱼ, risk(hᵢ)/risk(hⱼ)=(f/pᵢ)/(fⱼ/pⱼ), where f and p denote, respectively, frequencies in the affected population and in the control population. While there is some power loss if the true model is not multiplicative, the loss tends to be mild except for extreme cases. Most importantly, p-values are always valid since they are computed with respect to null hypothesis.

An association signal detected in one association study may be replicated in a second cohort, for example a cohort from a different population (e.g., different region of same country, or a different country) of the same or different ethnicity. The advantage of replication studies is that the number of tests performed in the replication study is usually quite small, and hence the less stringent the statistical measure that needs to be applied. For example, for a genome-wide search for susceptibility variants for a particular disease or trait using 300,000 SNPs, a correction for the 300,000 tests performed (one for each SNP) can be performed. Since many SNPs on the arrays typically used are correlated (i.e., in LD), they are not independent. Thus, the correction is conservative. Nevertheless, applying this correction factor requires an observed P-value of less than 0.05/300,000=1.7×10⁻⁷ for the signal to be considered significant applying this conservative test on results from a single study cohort. Obviously, signals found in a genome-wide association study with P-values less than this conservative threshold (i.e., more significant) are a measure of a true genetic effect, and replication in additional cohorts is not necessary from a statistical point of view. Importantly, however, signals with P-values that are greater than this threshold may also be due to a true genetic effect. The sample size in the first study may not have been sufficiently large to provide an observed P-value that meets the conservative threshold for genome-wide significance, or the first study may not have reached genome-wide significance due to inherent fluctuations due to sampling. Since the correction factor depends on the number of statistical tests performed, if one signal (one SNP) from an initial study is replicated in a second case-control cohort, the appropriate statistical test for significance is that for a single statistical test, i.e., P-value less than 0.05. Replication studies in one or even several additional case-control cohorts have the added advantage of providing assessment of the association signal in additional populations, thus simultaneously confirming the initial finding and providing an assessment of the overall significance of the genetic variant(s) being tested in human populations in general.

The results from several case-control cohorts can also be combined to provide an overall assessment of the underlying effect. The methodology commonly used to combine results from multiple genetic association studies is the Mantel-Haenszel model (Mantel and Haenszel, J Natl Cancer Inst 22:719-48 (1959)). The model is designed to deal with the situation where association results from different populations, with each possibly having a different population frequency of the genetic variant, are combined. The model combines the results assuming that the effect of the variant on the risk of the disease, a measured by the OR or RR, is the same in all populations, while the frequency of the variant may differ between the populations. Combining the results from several populations has the added advantage that the overall power to detect a real underlying association signal is increased, due to the increased statistical power provided by the combined cohorts. Furthermore, any deficiencies in individual studies, for example due to unequal matching of cases and controls or population stratification will tend to balance out when results from multiple cohorts are combined, again providing a better estimate of the true underlying genetic effect.

### Risk Assessment and Diagnostics

Within any given population, there is an absolute risk of developing a disease or trait, defined as the chance of a person developing the specific disease or trait over a specified time-period. For example, a woman's lifetime absolute risk of breast cancer is one in nine. That is to say, one woman in every nine will develop breast cancer at some point in their lives. Risk is typically measured by looking at very large numbers of people, rather than at a particular individual. Risk is often presented in terms of Absolute Risk (AR) and Relative Risk (RR). Relative Risk is used to compare risks associating with two variants or the risks of two different groups of people. For example, it can be used to compare a group of people with a certain genotype with another group having a different genotype. For a disease, a relative risk of 2 means that one group has twice the chance of developing a disease as the other group. The risk presented is usually the relative risk for a person, or a specific genotype of a person, compared to the population with matched gender and ethnicity. Risks of two individuals of the same gender and ethnicity could be compared in a simple manner. For example, if, compared to the population, the first individual has relative risk 1.5 and the second has relative risk 0.5, then the risk of the first individual compared to the second individual is 1.5/0.5=3.

### Risk Calculations

The creation of a model to calculate the overall genetic risk involves two steps: i) conversion of odds-ratios for a single genetic variant into relative risk and ii) combination of risk from multiple variants in different genetic loci into a single relative risk value.

Deriving Risk from Odds-Ratios

Most gene discovery studies for complex diseases that have been published to date in authoritative journals have employed a case-control design because of their retrospective setup. These studies sample and genotype a selected set of cases (people who have the specified disease condition) and control individuals. The interest is in genetic variants (alleles) which frequency in cases and controls differ significantly.

The results are typically reported in odds ratios, that is the ratio between the fraction (probability) with the risk variant (carriers) versus the non-risk variant (non-carriers) in the groups of affected versus the controls, i.e. expressed in terms of probabilities conditional on the affection status:

OR=(Pr(c|A)/Pr(nc|A))/(Pr(c|C)/Pr(nc|C))

Sometimes it is however the absolute risk for the disease that we are interested in, i.e. the fraction of those individuals carrying the risk variant who get the disease or in other words the probability of getting the disease. This number cannot be directly measured in case-control studies, in part, because the ratio of cases versus controls is typically not the same as that in the general population. However, under certain assumption, we can estimate the risk from the odds ratio.

It is well known that under the rare disease assumption, the relative risk of a disease can be approximated by the odds ratio. This assumption may however not hold for many common diseases. Still, it turns out that the risk of one genotype variant relative to another can be estimated from the odds ratio expressed above. The calculation is particularly simple under the assumption of random population controls where the controls are random samples from the same population as the cases, including affected people rather than being strictly unaffected individuals. To increase sample size and power, many of the large genome-wide association and replication studies use controls that were neither age-matched with the cases, nor were they carefully scrutinized to ensure that they did not have the disease at the time of the study. Hence, while not exactly, they often approximate a random sample from the general population. It is noted that this assumption is rarely expected to be satisfied exactly, but the risk estimates are usually robust to moderate deviations from this assumption.

Calculations show that for the dominant and the recessive models, where we have a risk variant carrier, “c”, and a non-carrier, “nc”, the odds ratio of individuals is the same as the risk ratio between these variants:

OR=Pr(A|c)/Pr(A|nc)=r

And likewise for the multiplicative model, where the risk is the product of the risk associated with the two allele copies, the allelic odds ratio equals the risk factor:

OR=Pr(A|aa)/Pr(A|ab)=Pr(A|ab)/Pr(A|bb)=r

Here “a” denotes the risk allele and “b” the non-risk allele. The factor “r” is therefore the relative risk between the allele types.

For many of the studies published in the last few years, reporting common variants associated with complex diseases, the multiplicative model has been found to summarize the effect adequately and most often provide a fit to the data superior to alternative models such as the dominant and recessive models.

The person skilled in the art will appreciate that for markers with two alleles present in the population being studied (such as SNPs), and wherein one allele is found in increased frequency in a group of individuals with a trait or disease in the population, compared with controls, the other allele of the marker will be found in decreased frequency in the group of individuals with the trait or disease, compared with controls. In such a case, one allele of the marker (the one found in increased frequency in individuals with the trait or disease) will be the at-risk allele, while the other allele will be a protective allele.

### Database

Determining susceptibility can alternatively or additionally comprise comparing nucleic acid sequence data and/or genotype data to a database containing correlation data between polymorphic markers and susceptibility to Bladder Cancer. The database can be part of a computer-readable medium as described herein.

In a specific aspect of the invention, the database comprises at least one measure of susceptibility to the condition for the polymorphic markers. For example, the database may comprise risk values associated with particular genotypes at such markers. The database may also comprise risk values associated with particular genotype combinations for multiple such markers.

In another specific aspect of the invention, the database comprises a look-up table containing at least one measure of susceptibility to the condition for the polymorphic markers.

### Further Steps

The methods disclosed herein can comprise additional steps which may occur before, after, or simultaneously with one of the aforementioned steps of the method of the invention. In a specific embodiment of the invention, the method of determining a susceptibility to Bladder Cancer further comprises reporting the susceptibility to at least one entity selected from the group consisting of the individual, a guardian of the individual, a genetic service provider, a physician, a medical organization, and a medical insurer. The reporting may be accomplished by any of several means. For example, the reporting can comprise sending a written report on physical media or electronically or providing an oral report to at least one entity of the group, which written or oral report comprises the susceptibility. Alternatively, the reporting can comprise providing the at least one entity of the group with a login and password, which provides access to a report comprising the susceptibility posted on a password-protected computer system.

### Study Population

In a general sense, the methods and kits described herein can be utilized from samples containing nucleic acid material (DNA or RNA) from any source and from any individual, or from genotype or sequence data derived from such samples. In preferred embodiments, the individual is a human individual. The individual can be an adult, child, or fetus. The nucleic acid source may be any sample comprising nucleic acid material, including biological samples, or a sample comprising nucleic acid material derived therefrom. The present invention also provides for assessing markers in individuals who are members of a target population. Such a target population is in one embodiment a population or group of individuals at risk of developing Bladder Cancer, based on other genetic factors, biophysical parameters, family history, etc.).

The Icelandic population is a Caucasian population of Northern European ancestry. A large number of studies reporting results of genetic linkage and association in the Icelandic population have been published in the last few years. Many of those studies show replication of variants, originally identified in the Icelandic population as being associating with a particular disease, in other populations (Sulem, P., et al. Nat Genet May 17, 2009 (Epub ahead of print); Rafnar, T., et al. Nat Genet. 41:221-7 (2009); Gretarsdottir, S., et al. Ann Neurol 64:402-9 (2008); Stacey, S, N., et al. Nat Genet. 40:1313-18 (2008); Gudbjartsson, D. F., et al. Nat Genet. 40:886-91 (2008); Styrkarsdottir, U., et al. N Engl J Med 358:2355-65 (2008); Thorgeirsson, T., et al. Nature 452:638-42 (2008); Gudmundsson, J., et al. Nat. Genet. 40:281-3 (2008); Stacey, S. N., et al. , Nat. Genet. 39:865-69 (2007); Helgadottir, A., et al. , Science 316:1491-93 (2007); Steinthorsdottir, V., et al. , Nat. Genet. 39:770-75 (2007); Gudmundsson, J., et al. , Nat. Genet. 39:631-37 (2007); Frayling, T M, Nature Reviews Genet. 8:657-662 (2007); Amundadottir, L. T., et al. , Nat. Genet. 38:652-58 (2006); Grant, S. F., et al. , Nat. Genet. 38:320-23 (2006)). Thus, genetic findings in the Icelandic population have in general been replicated in other populations, including populations from Africa and Asia.

It is thus believed that the markers described herein to be associated with risk of Bladder Cancer will show similar association in other human populations. Particular embodiments comprising individual human populations are thus also contemplated and within the scope of the invention.

Such embodiments relate to human subjects that are from one or more human population including, but not limited to, Caucasian populations, European populations, American populations, Eurasian populations, Asian populations, Central/South Asian populations, East Asian populations, Middle

The racial contribution in individual subjects may also be determined by genetic analysis. Genetic analysis of ancestry may be carried out using unlinked microsatellite markers such as those set out in Smith et al. (Am J Hum Genet. 74, 1001-13 (2004)).

In certain embodiments, the invention relates to markers identified in specific populations, as described in the above. The person skilled in the art will appreciate that measures of linkage disequilibrium (LD) may give different results when applied to different populations. This is due to different population history of different human populations as well as differential selective pressures that may have led to differences in LD in specific genomic regions. It is also well known to the person skilled in the art that certain markers, e.g. SNP markers, have different population frequency in different populations, or are polymorphic in one population but not in another. The person skilled in the art will however apply the methods available and as taught herein to practice the present invention in any given human population. This may include assessment of polymorphic markers in the LD region of the present invention, so as to identify those markers that give strongest association within the specific population. Thus, the at-risk variants of the present invention may reside on different haplotype background and in different frequencies in various human populations. However, utilizing methods known in the art and the markers of the present invention, the invention can be practiced in any given human population.

### Screening Methods

The invention also provides a method of screening candidate markers for assessing susceptibility to Bladder Cancer. The invention also provides a method of identification of a marker for use in assessing susceptibility to Bladder Cancer. The method may comprise analyzing the frequency of at least one allele of a polymorphic marker in a population of human individuals diagnosed with Bladder Cancer, wherein a significant difference in frequency of the at least one allele in the population of human individuals diagnosed with Bladder Cancer as compared to the frequency of the at least one allele in a control population of human individuals is indicative of the allele as a marker of the Bladder Cancer. In certain embodiments, the candidate marker is a marker in linkage disequilibrium with rs1058396.

In one embodiment, the method comprises (i) identifying at least one polymorphic marker within the human SLC14A1 gene; (ii) obtaining sequence information about the at least one polymorphic marker in a group of individuals diagnosed with Bladder Cancer; and (iii) obtaining sequence information about the at least one polymorphic marker in a group of control individuals; wherein determination of a significant difference in frequency of at least one allele in the at least one polymorphism in individuals diagnosed with Bladder Cancer as compared with the frequency of the at least one allele in the control group is indicative of the at least one polymorphism being useful for assessing susceptibility to Bladder Cancer. In certain embodiments, the marker is in linkage disequilibrium with rs1058396.

In one embodiment, an increase in frequency of the at least one allele in the at least one polymorphism in individuals diagnosed with Bladder Cancer, as compared with the frequency of the at least one allele in the control group, is indicative of the at least one polymorphism being useful for assessing increased susceptibility to Bladder Cancer. In another embodiment, a decrease in frequency of the at least one allele in the at least one polymorphism in individuals diagnosed with Bladder Cancer, as compared with the frequency of the at least one allele in the control group, is indicative of the at least one polymorphism being useful for assessing decreased susceptibility to, or protection against, Bladder Cancer.

### Utility of Genetic Testing

The person skilled in the art will appreciate and understand that the variants described herein in general do not, by themselves, provide an absolute identification of individuals who will develop urinary bladder cancer. The variants described herein do however indicate increased and/or decreased likelihood that individuals carrying the at-risk or protective variants of the invention will develop UBC, or symptoms associated with UBC. This information is however extremely valuable in itself, as outlined in more detail in the below, as it can be used to, for example, initiate preventive measures at an early stage, perform regular physical exams to monitor the progress and/or appearance of symptoms, or to schedule exams at a regular interval to identify early symptoms, so as to be able to apply treatment at an early stage.

Bladder cancer is a disease with a high prevalence and potential for improved survival with early detection. Understanding of the genetic factors contributing to the residual genetic risk for bladder cancer is very limited. No universally successful method for the prevention or treatment of bladder cancer is currently available. Management of the disease currently relies on a combination of early diagnosis, appropriate treatments and secondary prevention. There are clear clinical imperatives for integrating genetic testing into all aspects of these management areas. Identification of cancer susceptibility genes may also reveal key molecular pathways that may be manipulated (e.g., using small or large molecular weight drugs) and may lead to more effective treatments.

A screening program that would result in detection of bladder cancer at an earlier stage, prior to muscle invasion or metastasis, could render a significant improvement in patient morbidity and overall survival. In order for bladder cancer screening to become a reality, first a high incidence population has to be identified and secondly a cost-effective marker with good performance characteristics has to be available. Individuals with many environmental risk factors, such as older male smokers and who have the high-risk genetic profile may benefit from periodic screening. Clinical screening for bladder cancer is mainly performed by urine cytology, cystoscopy or Hematuria tests.

Home urine dipstick to assess for hematuria is convenient, inexpensive, and noninvasive. However, utility of widespread screening with hematuria testing is limited due to the low positive predictive value (PPV) of the test. The PPV for hematuria dipstick for screening ranges between 5 and 8.3% resulting in many unnecessary workups with their attendant patient anxiety and cost. Due to the relatively low sensitivity and low PPV of the reagent strip for hemoglobin as well as cytology, multiple urine-based bladder markers have been developed to try to assist in detecting bladder cancer non-invasively (Lotan Y, Roehrborn C G (2003) Urology 61(1):109-118). These include the NMP22 BladderChek Test (Matritech Inc., Newton, Mass., USA) and UroVysion (Vysis Downer's Grove, Ill., USA) (Grossman, H B et al. JAMA 293:810-816, 2005).

Genetic variants described herein can be used alone or in combination, as well as in combination with other factors, including other genetic risk factors or biomarkers, for risk assessment of an individual for UBC. In certain embodiments, the variants described herein may be included in genetic screening programs for UBC that also include other risk factors for UBC, including variants on chromosome 3q (e.g, rs710521), chromosome 4p (e.g., rs798766), chromosome 5q (e.g., rs4446484) and chromosome 8q (e.g., rs9642880). Other factors known to affect the predisposition of an individual towards developing risk of developing UBC are also known to the person skilled in the art and can be utilized in such assessment. These include, but are not limited to, age, gender, smoking status and/or smoking history, family history of cancer, and of UBC in particular. Methods known in the art can be used for such assessment, including multivariate analyses or logistic regression.

### Diagnostic Methods

Polymorphic markers associated with increased susceptibility of Bladder Cancer and related conditions are useful in diagnostic methods. While methods of diagnosing such conditions are known in the art, the detection of one or more alleles of the specific polymorphic markers advantageously may be useful for detection of these conditions at their early stages and may also reduce the occurrence of mis-diagnosis. In this regard, the invention further provides methods of diagnosing these conditions comprising obtaining sequence data identifying at least one allele of at least one polymorphic marker of a specified group, in conjunction with carrying out one or more steps, e.g., clinical diagnostic steps, such as any of those described herein.

The present invention pertains in some embodiments to methods of clinical applications of diagnosis, e.g., diagnosis performed by a medical professional. In other embodiments, the invention pertains to methods of diagnosis or methods of determination of a susceptibility performed by a layman. The layman can be the customer of a sequencing or genotyping service. The layman may also be a genotype or sequencing service provider, who performs analysis on a DNA sample from an individual, in order to provide service related to genetic risk factors for particular traits or diseases, based on the genotype status of the individual (i.e., the customer). Sequencing methods include for example those discussed in the above, but in general any suitable sequencing method may be used in the methods described and claimed herein. Recent technological advances in genotyping technologies, including high-throughput genotyping of SNP markers, such as Molecular Inversion Probe array technology (e.g., Affymetrix GeneChip), and BeadArray Technologies (e.g., Illumina GoldenGate and Infinium assays) have made it possible for individuals to have their own genome assessed for up to one million SNPs simultaneously, at relatively little cost. The resulting genotype information, which can be made available to the individual, can be compared to information about disease or trait risk associated with various SNPs, including information from public literature and scientific publications.

The application of disease-associated alleles as described herein, can thus for example be performed by the individual, through analysis of his/her genotype data, by a health professional based on results of a clinical test, or by a third party, including the genotype or sequencing service provider. The third party may also be service provider who interprets genotype or sequence information from the customer to provide service related to specific genetic risk factors, including the genetic markers described herein. In other words, the diagnosis or determination of a susceptibility of genetic risk can be made by health professionals, genetic counselors, third parties providing genotyping and/or sequencing service, third parties providing risk assessment service or by the layman (e.g., the individual), based on information about the genotype status of an individual and knowledge about the risk conferred by particular genetic risk factors (e.g., particular SNPs). In the present context, the term “diagnosing”, “diagnose a susceptibility” and “determine a susceptibility” is meant to refer to any available method for determining a susceptibility or risk of disease, including those mentioned above.

In certain embodiments, a sample containing genomic DNA from an individual is collected. Such sample can for example be a buccal swab, a saliva sample, a blood sample, or other suitable samples containing genomic DNA, as described further herein. In certain embodiments, the sample is obtained by non-invasive means (e.g., for obtaining a buccal sample, saliva sample, hair sample or skin sample). In certain embodiments, the sample is obtained by non-surgical means, i.e. in the absence of a surgical intervention on the individual that puts the individual at substantial health risk. Such embodiments may, in addition to non-invasive means also include obtaining sample by extracting a blood sample (e.g., a venous blood sample). The genomic DNA obtained from the individual is then analyzed using any common technique available to the skilled person, such as high-throughput technologies for genotyping and/or sequencing. Results from such methods are stored in a convenient data storage unit, such as a data carrier, including computer databases, data storage disks, or by other convenient data storage means. In certain embodiments, the computer database is an object database, a relational database or a post-relational database. The genotype data is subsequently analyzed for the presence of certain variants known to be susceptibility variants for a particular human condition, such as the genetic variants described herein associated with risk of Bladder Cancer. Genotype and/or sequencing data can be retrieved from the data storage unit using any convenient data query method. Calculating risk conferred by a particular genotype for the individual can be based on comparing the genotype of the individual to previously determined risk (expressed as a relative risk (RR) or and odds ratio (OR), for example) for the genotype, for example for an heterozygous carrier of an at-risk variant. The calculated risk for the individual can be the relative risk for a person, or for a specific genotype of a person, compared to the average population with matched gender and ethnicity. The average population risk can be expressed as a weighted average of the risks of different genotypes, using results from a reference population, and the appropriate calculations to calculate the risk of a genotype group relative to the population can then be performed. Alternatively, the risk for an individual is based on a comparison of particular genotypes, for example heterozygous carriers of an at-risk allele of a marker compared with non-carriers of the at-risk allele. The calculated risk estimated can be made available to the customer via a website, preferably a secure website.

In certain embodiments, a service provider will include in the provided service all of the steps of isolating genomic DNA from a sample provided by the customer, performing genotyping or sequencing of the isolated DNA, calculating genetic risk based on the genotype or sequence data, and report the risk to the customer. In some other embodiments, the service provider will include in the service the interpretation of genotype data for the individual, i.e., risk estimates for particular genetic variants based on the genotype data for the individual. In some other embodiments, the service provider may include service that includes genotyping and/or sequencing service and interpretation of the resulting sequence data, starting from a sample of isolated DNA from the individual.

Decreased susceptibility is in general determined based on the absence of particular at-risk alleles and/or the presence of protective alleles. As discussed in more detail herein, for biallelic markers such as SNPs, the alternate allele of an at-risk allele is, by definition, a protective allele. Determinations of its presence, in particular for homozygous individuals, is thus indicative of a decreased susceptibility.

### Kits

Kits useful in the methods of the invention comprise components useful in any of the methods described herein, including for example, primers for nucleic acid amplification, hybridization probes, restriction enzymes (e.g., for RFLP analysis), allele-specific oligonucleotides, antibodies that bind to specific polypeptides encoded by a nucleic acid of the invention (e.g., N280D, R4W, M 167 and/or K44E SLC14A1 polypeptides), means for amplification of a nucleic acids, means for analyzing the nucleic acid sequence of a nucleic acids, means for analyzing the amino acid sequence of a polypeptide, etc. The kits can for example include necessary buffers, nucleic acid primers for amplifying nucleic acids of the invention (e.g., a nucleic acid segment comprising one or more of the polymorphic markers as described herein), and reagents for allele-specific detection of the fragments amplified using such primers and necessary enzymes (e.g., DNA polymerase). Additionally, kits can provide reagents for assays to be used in combination with other diagnostic assays for UBC.

In one embodiment, the invention pertains to a kit for assaying a sample from a subject to detect a susceptibility to UBC in a subject, wherein the kit comprises reagents necessary for selectively detecting at least one allele of at least one polymorphism of the present invention in the genome of the individual. In a particular embodiment, the reagents comprise at least one contiguous oligonucleotide that hybridizes to a fragment of the genome of the individual comprising at least one polymorphism of the present invention. In another embodiment, the reagents comprise at least one pair of oligonucleotides that hybridize to opposite strands of a genomic segment obtained from a subject, wherein each oligonucleotide primer pair is designed to selectively amplify a fragment of the genome of the individual that includes at least one polymorphism associated with UBC risk. In one such embodiment, the polymorphism is selected from the polymorphic markers rs1058396, rs11877062, rs2298719 and rs2298720, and markers in linkage disequilibrium therewith. In yet another embodiment, the fragment is at least 20 base pairs in size. In another embodiment, the fragment is no more than 200 base pairs in size. Such oligonucleotides or nucleic acids (e.g., oligonucleotide primers) can be designed using portions of the nucleic acid sequence flanking the polymorphic site. In another embodiment, the kit comprises one or more labeled nucleic acids capable of allele-specific detection of one or more specific polymorphic markers or haplotypes, and reagents for detection of the label. Suitable labels include, e.g., a radioisotope, a fluorescent label, an enzyme label, an enzyme co-factor label, a magnetic label, a spin label, an epitope label.

In a preferred embodiment, the DNA template containing the SNP polymorphism is amplified by Polymerase Chain Reaction (PCR) prior to detection, and primers for such amplification are included in the reagent kit. In such an embodiment, the amplified DNA serves as the template for the detection probe and the enhancer probe.

In one embodiment, the DNA template is amplified by means of Whole Genome Amplification (WGA) methods, prior to assessment for the presence of specific polymorphic markers as described herein. Standard methods well known to the skilled person for performing WGA may be utilized, and are within scope of the invention. In one such embodiment, reagents for performing WGA are included in the reagent kit.

In certain embodiments, the kit further comprises a collection of data comprising correlation data between the polymorphic markers assessed by the kit and susceptibility to urinary bladder cancer.

In a further aspect of the present invention, a pharmaceutical pack (kit) is provided, the pack comprising a therapeutic agent and a set of instructions for administration of the therapeutic agent to humans diagnostically tested for one or more variants of the present invention, as disclosed herein. The therapeutic agent can be a small molecule drug, an antibody, a peptide, an antisense or RNAi molecule, or other therapeutic molecules. In one embodiment, an individual identified as a carrier of at least one variant of the present invention is instructed to take a prescribed dose of the therapeutic agent. In one such embodiment, an individual identified as a homozygous carrier of at least one variant of the present invention is instructed to take a prescribed dose of the therapeutic agent. In another embodiment, an individual identified as a non-carrier of at least one variant of the present invention is instructed to take a prescribed dose of the therapeutic agent.

In certain embodiments, the kit further comprises a set of instructions for using the reagents comprising the kit.

### Antisense Agents

The nucleic acids and/or variants described herein, or nucleic acids comprising their complementary sequence, may be used as antisense constructs to control gene expression in cells, tissues or organs. The methodology associated with antisense techniques is well known to the skilled artisan, and is for example described and reviewed in AntisenseDrug Technology: Principles, Strategies, and Applications, Crooke, ed., Marcel Dekker Inc., New York (2001).

In general, antisense agents (antisense oligonucleotides) are comprised of single stranded oligonucleotides (RNA or DNA) that are capable of binding to a complimentary nucleotide segment.

By binding the appropriate target sequence, an RNA-RNA, DNA-DNA or RNA-DNA duplex is formed. The antisense oligonucleotides are complementary to the sense or coding strand of a gene. It is also possible to form a triple helix, where the antisense oligonucleotide binds to duplex DNA.

Several classes of antisense oligonucleotide are known to those skilled in the art, including cleavers and blockers. The former bind to target RNA sites, activate intracellular nucleases (e.g., RnaseH or Rnase L), that cleave the target RNA. Blockers bind to target RNA, inhibit protein translation by steric hindrance of the ribosomes. Examples of blockers include nucleic acids, morpholino compounds, locked nucleic acids and methylphosphonates (Thompson, Drug Discovery Today, 7:912-917 (2002)). Antisense oligonucleotides are useful directly as therapeutic agents, and are also useful for determining and validating gene function, for example by gene knock-out or gene knock-down experiments. Antisense technology is further described in Lavery et al. , Curr. Opin. Drug Discov. Devel. 6:561-569 (2003), Stephens et al. , Curr. Opin. Mol. Ther. 5:118-122 (2003), Kurreck, Eur. J. Biochem. 270:1628-44 (2003), Dias et al. , Mol. Cancer. Ter. 1:347-55 (2002), Chen, Methods Mol. Med. 75:621-636 (2003), Wang et al. , Curr. Cancer Drug Targets 1:177-96 (2001), and Bennett, Antisense Nucleic Acid Drug. Dev. 12:215-24 (2002).

In certain embodiments, the antisense agent is an oligonucleotide that is capable of binding to a particular nucleotide segment. In certain embodiments, the nucleotide segment comprises all or a portion of the human SLC14A1 gene. In certain other embodiments, the antisense nucleotide is capable of binding to a nucleotide segment of the human SLC14A1 as set forth in SEQ ID NO:134. Antisense nucleotides can be from 5-400 nucleotides in length, including 5-200 nucleotides, 5-100 nucleotides, 10-50 nucleotides, and 10-30 nucleotides. In certain preferred embodiments, the antisense nucleotides are from 14-50 nucleotides in length, including 14-40 nucleotides and 14-30 nucleotides.

The variants described herein can also be used for the selection and design of antisense reagents that are specific for particular variants. Using information about the variants described herein, antisense oligonucleotides or other antisense molecules that specifically target mRNA molecules that contain one or more variants of the invention can be designed. In this manner, expression of mRNA molecules that contain one or more variant of the present invention (e.g. at risk marker alleles, such as rs1058396 allele G, rs11877062 allele C, rs2298720 allele G and rs2298719 allele A) can be inhibited or blocked. In one embodiment, the antisense molecules are designed to specifically bind a particular allelic form (e.g., an at-risk variant) of the target nucleic acid, thereby inhibiting translation of a product originating from this specific allele or haplotype, but which do not bind other or alternate variants at the specific polymorphic sites of the target nucleic acid molecule. As antisense molecules can be used to inactivate mRNA so as to inhibit gene expression, and thus protein expression, the molecules can be used for disease treatment. The methodology can involve cleavage by means of ribozymes containing nucleotide sequences complementary to one or more regions in the mRNA that attenuate the ability of the mRNA to be translated. Such mRNA regions include, for example, protein-coding regions, in particular protein-coding regions corresponding to catalytic activity, substrate and/or ligand binding sites, or other functional domains of a protein.

The phenomenon of RNA interference (RNAi) has been actively studied for the last decade, since its original discovery in C. elegans (Fire et al. , Nature 391:806-11 (1998)), and in recent years its potential use in treatment of human disease has been actively pursued (reviewed in Kim & Rossi, Nature Rev. Genet. 8:173-204 (2007)). RNA interference (RNAi), also called gene silencing, is based on using double-stranded RNA molecules (dsRNA) to turn off specific genes. In the cell, cytoplasmic double-stranded RNA molecules (dsRNA) are processed by cellular complexes into small interfering RNA (siRNA). The siRNA guide the targeting of a protein-RNA complex to specific sites on a target mRNA, leading to cleavage of the mRNA (Thompson, Drug Discovery Today, 7:912-917 (2002)). The siRNA molecules are typically about 20, 21, 22 or 23 nucleotides in length. Thus, one aspect of the invention relates to isolated nucleic acid molecules, and the use of those molecules for RNA interference, i.e. as small interfering RNA molecules (siRNA). In one embodiment, the isolated nucleic acid molecules are 18-26 nucleotides in length, preferably 19-25 nucleotides in length, more preferably 20-24 nucleotides in length, and more preferably 21, 22 or 23 nucleotides in length.

Another pathway for RNAi-mediated gene silencing originates in endogenously encoded primary microRNA (pri-miRNA) transcripts, which are processed in the cell to generate precursor miRNA (pre-miRNA). These miRNA molecules are exported from the nucleus to the cytoplasm, where they undergo processing to generate mature miRNA molecules (miRNA), which direct translational inhibition by recognizing target sites in the 3′ untranslated regions of mRNAs, and subsequent mRNA degradation by processing P-bodies (reviewed in Kim & Rossi, Nature Rev. Genet. 8:173-204 (2007)).

Clinical applications of RNAi include the incorporation of synthetic siRNA duplexes, which preferably are approximately 20-23 nucleotides in size, and preferably have 3′ overlaps of 2 nucleotides. Knockdown of gene expression is established by sequence-specific design for the target mRNA. Several commercial sites for optimal design and synthesis of such molecules are known to those skilled in the art.

Other applications provide longer siRNA molecules (typically 25-30 nucleotides in length, preferably about 27 nucleotides), as well as small hairpin RNAs (shRNAs; typically about 29 nucleotides in length). The latter are naturally expressed, as described in Amarzguioui et al. (FEBS Lett. 579:5974-81 (2005)). Chemically synthetic siRNAs and shRNAs are substrates for in vivo processing, and in some cases provide more potent gene-silencing than shorter designs (Kim et al. , Nature Biotechnol. 23:222-226 (2005); Siolas et al. , Nature Biotechnol. 23:227-231 (2005)). In general siRNAs provide for transient silencing of gene expression, because their intracellular concentration is diluted by subsequent cell divisions. By contrast, expressed shRNAs mediate long-term, stable knockdown of target transcripts, for as long as transcription of the shRNA takes place (Marques et al. , Nature Biotechnol. 23:559-565 (2006); Brummelkamp et al. , Science 296: 550-553 (2002)).

Since RNAi molecules, including siRNA, miRNA and shRNA, act in a sequence-dependent manner, the variants presented herein can be used to design RNAi reagents that recognize specific nucleic acid molecules comprising specific alleles and/or haplotypes (e.g., the alleles and/or haplotypes of the present invention), while not recognizing nucleic acid molecules comprising other alleles or haplotypes. These RNAi reagents can thus recognize and destroy the target nucleic acid molecules. As with antisense reagents, RNAi reagents can be useful as therapeutic agents (i.e., for turning off disease-associated genes or disease-associated gene variants), but may also be useful for characterizing and validating gene function (e.g., by gene knock-out or gene knock-down experiments).

Delivery of RNAi may be performed by a range of methodologies known to those skilled in the art. Methods utilizing non-viral delivery include cholesterol, stable nucleic acid-lipid particle (SNALP), heavy-chain antibody fragment (Fab), aptamers and nanoparticles. Viral delivery methods include use of lentivirus, adenovirus and adeno-associated virus. The siRNA molecules are in some embodiments chemically modified to increase their stability. This can include modifications at the 2′ position of the ribose, including 2′-O-methylpurines and 2′-fluoropyrimidines, which provide resistance to Rnase activity. Other chemical modifications are possible and known to those skilled in the art.

The following references provide a further summary of RNAi, and possibilities for targeting specific genes using RNAi: Kim & Rossi, Nat. Rev. Genet. 8:173-184 (2007), Chen & Rajewsky, Nat. Rev. Genet. 8: 93-103 (2007), Reynolds, et al., Nat. Biotechnol. 22:326-330 (2004), Chi et al., Proc. Natl. Acad. Sci. USA 100:6343-6346 (2003), Vickers et al., J. Biol. Chem. 278:7108-7118 (2003), Agami, Curr. Opin. Chem. Biol. 6:829-834 (2002), Lavery, et al., Curr. Opin. Drug Discov. Devel. 6:561-569 (2003), Shi, Trends Genet. 19:9-12 (2003), Shuey et al., Drug Discov. Today 7:1040-46 (2002), McManus et al., Nat. Rev. Genet. 3:737-747 (2002), Xia et al., Nat. Biotechnol. 20:1006-10 (2002), Plasterk et al., curr. Opin. Genet. Dev. 10:562-7 (2000), Bosher et al., Nat. Cell Biol. 2:E31-6 (2000), and Hunter, Curr. Biol. 9:R440—442 (1999)

### Methods of Assessing Probability of Response to Therapeutic Agents, Methods of Monitoring Progress Of Treatment and Methods of Treatment

As is known in the art, individuals can have differential responses to a particular therapy (e.g., a therapeutic agent or therapeutic method). Pharmacogenomics addresses the issue of how genetic variations (e.g., the variants (markers and/or haplotypes) of the present invention) affect drug response, due to altered drug disposition and/or abnormal or altered action of the drug. Thus, the basis of the differential response may be genetically determined in part. Clinical outcomes due to genetic variations affecting drug response may result in toxicity of the drug in certain individuals (e.g., carriers or non-carriers of the genetic variants of the present invention), or therapeutic failure of the drug. Therefore, the variants of the present invention may determine the manner in which a therapeutic agent and/or method acts on the body, or the way in which the body metabolizes the therapeutic agent.

Accordingly, in one embodiment, the presence of a particular allele at a polymorphic site is indicative of a different response, e.g. a different response rate, to a particular treatment modality. This means that a patient diagnosed with UBC, and carrying a certain allele at a polymorphic site described herein (e.g., the at-risk and protective alleles of the invention) would respond better to, or worse to, a specific therapeutic, drug and/or other therapy used to treat the disease. Therefore, the identity of a marker allele could aid in deciding what treatment should be used for a the patient. For example, for a newly diagnosed patient, the presence of an at-risk marker allele of the present invention may be assessed (e.g., through testing DNA derived from a blood sample, as described herein). If the patient is positive for the marker allele, then the physician recommends one particular therapy, while if the patient is negative for the at least one allele of a marker, or a haplotype, then a different course of therapy may be recommended. Thus, the patient's carrier status could be used to help determine whether a particular treatment modality should be administered.

As described above, current clinical treatment options for UBC include different surgical procedures, depending on the severity of the cases, e.g. whether the cancer is invasive into the muscle wall of the bladder. Treatment options also include radiation therapy, for which a proportion of patients experience adverse symptoms. The markers of the invention, as described herein, may be used to assess response to these therapeutic options, or to predict the progress of therapy using any one of these treatment options. Thus, genetic profiling can be used to select the appropriate treatment strategy based on the genetic status of the individual, or it may be used to predict the outcome of the particular treatment option, and thus be useful in the strategic selection of treatment options or a combination of available treatment options. Again, such profiling and classification of individuals is supported further by first analysing known groups of patients for marker and/or haplotype status, as described further herein.

The present invention also relates to methods of monitoring progress or effectiveness of a treatment for urinary bladder cancer. This can be done based on the genotype status of the markers described herein, i.e., by assessing the absence or presence of at least one allele of at least one polymorphic marker as disclosed herein, or by monitoring expression of genes that are associated with the variants (markers and haplotypes) described herein (e.g., the SLC14A1 gene). The risk gene mRNA or the encoded polypeptide can be measured in a tissue sample (e.g., a peripheral blood sample, or a biopsy sample). Expression levels and/or mRNA levels can thus be determined before and during treatment to monitor its effectiveness. Alternatively, or concomitantly, the genotype status of at least one risk variant for UBC as presented herein is determined before and during treatment to monitor its effectiveness.

In a further aspect, the markers of the present invention can be used to increase power and effectiveness of clinical trials. Thus, individuals who are carriers of at-risk variants described herein may be more likely to respond favorably to a particular treatment modality for Bladder Cancer. In one embodiment, individuals who carry an at-risk variant are more likely to be responders to the treatment. In another embodiment, individuals who carry at-risk variants of a gene, which expression and/or function is altered by the at-risk variant (e.g., the at-risk missense variants in the SLC14A1 described herein), are more likely to be responders to a treatment modality targeting that gene, its expression or its gene product. This application can improve the safety of clinical trials, but can also enhance the chance that a clinical trial will demonstrate statistically significant efficacy, which may be limited to a certain sub-group of the population. Thus, one possible outcome of such a trial is that carriers of certain genetic variants, e.g., at-risk markers described herein, are statistically significantly likely to show positive response to the therapeutic agent, i.e. experience alleviation of symptoms associated with Bladder Cancer when taking the therapeutic agent or drug as prescribed.

### Computer-Implemented Aspects

As understood by those of ordinary skill in the art, the methods and information described herein may be implemented, in all or in part, as computer executable instructions on known computer readable media. For example, the methods described herein may be implemented in hardware. Alternatively, the method may be implemented in software stored in, for example, one or more memories or other computer readable medium and implemented on one or more processors. As is known, the processors may be associated with one or more controllers, calculation units and/or other units of a computer system, or implanted in firmware as desired. If implemented in software, the routines may be stored in any computer readable memory such as in RAM, ROM, flash memory, a magnetic disk, a laser disk, or other storage medium, as is also known. Likewise, this software may be delivered to a computing device via any known delivery method including, for example, over a communication channel such as a telephone line, the Internet, a wireless connection, etc., or via a transportable medium, such as a computer readable disk, flash drive, etc.

More generally, and as understood by those of ordinary skill in the art, the various steps described above may be implemented as various blocks, operations, tools, modules and techniques which, in turn, may be implemented in hardware, firmware, software, or any combination of hardware, firmware, and/or software. When implemented in hardware, some or all of the blocks, operations, techniques, etc. may be implemented in, for example, a custom integrated circuit (IC), an application specific integrated circuit (ASIC), a field programmable logic array (FPGA), a programmable logic array (PLA), etc.

When implemented in software, the software may be stored in any known computer readable medium such as on a magnetic disk, an optical disk, or other storage medium, in a RAM or ROM or flash memory of a computer, processor, hard disk drive, optical disk drive, tape drive, etc. Likewise, the software may be delivered to a user or a computing system via any known delivery method including, for example, on a computer readable disk or other transportable computer storage mechanism.

Thus, another aspect of the invention is a system that is capable of carrying out a part or all of a method of the invention, or carrying out a variation of a method of the invention as described herein in greater detail. Exemplary systems include, as one or more components, computing systems, environments, and/or configurations that may be suitable for use with the methods and include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like. In some variations, a system of the invention includes one or more machines used for analysis of biological material (e.g., genetic material), as described herein. In some variations, this analysis of the biological material involves a chemical analysis and/or a nucleic acid amplification.

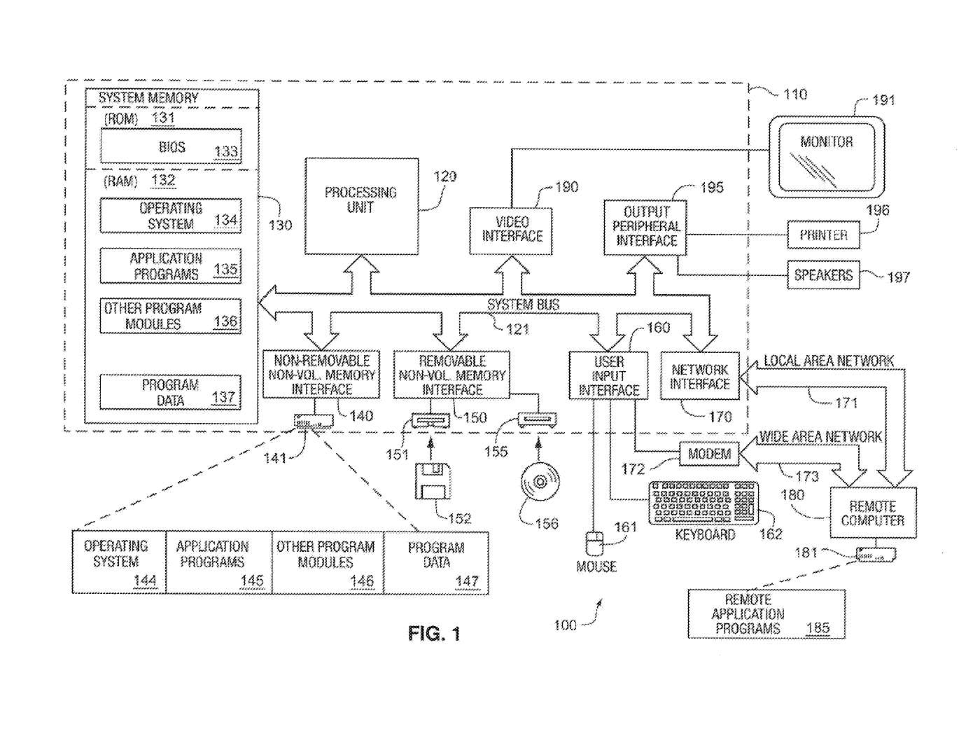

FIG. 1 illustrates an example of a suitable computing system environment 100 on which a system for the steps of the claimed method and apparatus may be implemented. The computing system environment 100 is only one example of a suitable computing environment and is not intended to suggest any limitation as to the scope of use or functionality of the method or apparatus of the claims. Neither should the computing environment 100 be interpreted as having any dependency or requirement relating to any one or combination of components illustrated in the exemplary operating environment 100.

The steps of the claimed method and system are operational with numerous other general purpose or special purpose computing system environments or configurations. Examples of well known computing systems, environments, and/or configurations that may be suitable for use with the methods or system of the claims include, but are not limited to, personal computers, server computers, hand-held or laptop devices, multiprocessor systems, microprocessor-based systems, set top boxes, programmable consumer electronics, network PCs, minicomputers, mainframe computers, distributed computing environments that include any of the above systems or devices, and the like.

The steps of the claimed method and system may be described in the general context of computer-executable instructions, such as program modules, being executed by a computer. Generally, program modules include routines, programs, objects, components, data structures, etc. that perform particular tasks or implement particular abstract data types. The methods and apparatus may also be practiced in distributed computing environments where tasks are performed by remote processing devices that are linked through a communications network. In both integrated and distributed computing environments, program modules may be located in both local and remote computer storage media including memory storage devices.

With reference to FIG. 1, an exemplary system for implementing the steps of the claimed method and system includes a general purpose computing device in the form of a computer 110. Components of computer 110 may include, but are not limited to, a processing unit 120, a system memory 130, and a system bus 121 that couples various system components including the system memory to the processing unit 120. The system bus 121 may be any of several types of bus structures including a memory bus or memory controller, a peripheral bus, and a local bus using any of a variety of bus architectures. By way of example, and not limitation, such architectures include Industry Standard Architecture (USA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

Computer 110 typically includes a variety of computer readable media. Computer readable media can be any available media that can be accessed by computer 110 and includes both volatile and nonvolatile media, removable and non-removable media. By way of example, and not limitation, computer readable media may comprise computer storage media and communication media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which can be used to store the desired information and which can accessed by computer 110. Communication media typically embodies computer readable instructions, data structures, program modules or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of the any of the above should also be included within the scope of computer readable media.

The system memory 130 includes computer storage media in the form of volatile and/or nonvolatile memory such as read only memory (ROM) 131 and random access memory (RAM) 132. A basic input/output system 133 (BIOS), containing the basic routines that help to transfer information between elements within computer 110, such as during start-up, is typically stored in ROM 131. RAM 132 typically contains data and/or program modules that are immediately accessible to and/or presently being operated on by processing unit 120. By way of example, and not limitation, FIG. 1 illustrates operating system 134, application programs 135, other program modules 136, and program data 137.

The computer 110 may also include other removable/non-removable, volatile/nonvolatile computer storage media. By way of example only, FIG. 1 illustrates a hard disk drive 140 that reads from or writes to non-removable, nonvolatile magnetic media, a magnetic disk drive 151 that reads from or writes to a removable, nonvolatile magnetic disk 152, and an optical disk drive 155 that reads from or writes to a removable, nonvolatile optical disk 156 such as a CD ROM or other optical media. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like. The hard disk drive 141 is typically connected to the system bus 121 through a non-removable memory interface such as interface 140, and magnetic disk drive 151 and optical disk drive 155 are typically connected to the system bus 121 by a removable memory interface, such as interface 150.

The drives and their associated computer storage media discussed above and illustrated in FIG. 1, provide storage of computer readable instructions, data structures, program modules and other data for the computer 110. In FIG. 1, for example, hard disk drive 141 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a keyboard 162 and pointing device 161, commonly referred to as a mouse, trackball or touch pad. Other input devices (not shown) may include a microphone, joystick, game pad, satellite dish, scanner, or the like. These and other input devices are often connected to the processing unit 120 through a user input interface 160 that is coupled to the system bus, but may be connected by other interface and bus structures, such as a parallel port, game port or a universal serial bus (USB). A monitor 191 or other type of display device is also connected to the system bus 121 via an interface, such as a video interface 190. In addition to the monitor, computers may also include other peripheral output devices such as speakers 197 and printer 196, which may be connected through an output peripheral interface 190.

The computer 110 may operate in a networked environment using logical connections to one or more remote computers, such as a remote computer 180. The remote computer 180 may be a personal computer, a server, a router, a network PC, a peer device or other common network node, and typically includes many or all of the elements described above relative to the computer 110, although only a memory storage device 181 has been illustrated in FIG. 1. The logical connections depicted in FIG. 1 include a local area network (LAN) 171 and a wide area network (WAN) 173, but may also include other networks. Such networking environments are commonplace in offices, enterprise-wide computer networks, intranets and the Internet.

When used in a LAN networking environment, the computer 110 is connected to the LAN 171 through a network interface or adapter 170. When used in a WAN networking environment, the computer 110 typically includes a modem 172 or other means for establishing communications over the WAN 173, such as the Internet. The modem 172, which may be internal or external, may be connected to the system bus 121 via the user input interface 160, or other appropriate mechanism. In a networked environment, program modules depicted relative to the computer 110, or portions thereof, may be stored in the remote memory storage device. By way of example, and not limitation, FIG. 1 illustrates remote application programs 185 as residing on memory device 181. It will be appreciated that the network connections shown are exemplary and other means of establishing a communications link between the computers may be used.

While the risk evaluation system and method, and other elements, have been described as preferably being implemented in software, they may be implemented in hardware, firmware, etc., and may be implemented by any other processor. Thus, the elements described herein may be implemented in a standard multi-purpose CPU or on specifically designed hardware or firmware such as an application-specific integrated circuit (ASIC) or other hard-wired device as desired, including, but not limited to, the computer 110 of FIG. 1. When implemented in software, the software routine may be stored in any computer readable memory such as on a magnetic disk, a laser disk, or other storage medium, in a RAM or ROM of a computer or processor, in any database, etc. Likewise, this software may be delivered to a user or a diagnostic system via any known or desired delivery method including, for example, on a computer readable disk or other transportable computer storage mechanism or over a communication channel such as a telephone line, the internet, wireless communication, etc. (which are viewed as being the same as or interchangeable with providing such software via a transportable storage medium).

Thus, many modifications and variations may be made in the techniques and structures described and illustrated herein without departing from the spirit and scope of the present invention. Thus, it should be understood that the methods and apparatus described herein are illustrative only and are not limiting upon the scope of the invention.

Accordingly, certain aspects of the invention relate to computer-implemented applications using the polymorphic markers and haplotypes described herein, and genotype and/or disease-association data derived therefrom. Such applications can be useful for storing, manipulating or otherwise analyzing genotype data that is useful in the methods of the invention. One example pertains to storing genotype and/or sequence data derived from an individual on readable media, so as to be able to provide the data to a third party (e.g., the individual, a guardian of the individual, a health care provider or genetic analysis service provider), or for deriving information from the data, e.g., by comparing the data to information about genetic risk factors contributing to increased susceptibility to Bladder Cancer, and reporting results based on such comparison.

In certain embodiments, computer-readable media suitably comprise capabilities of storing (i) identifier information for at least one polymorphic marker (e.g, marker names), as described herein; (ii) an indicator of the identity (e.g., presence or absence) of at least one allele of said at least one marker in individuals with the disease (e.g., rs1058396, rs11877062, rs2298720 or rs2298719, or the encoded protein variants); and (iii) an indicator of the risk associated with a particular marker allele (e.g., the G allele of rs1058396). The media may also be suitably comprise capabilities of storing protein sequence data.

In one embodiment, the invention provides a computer-readable medium having computer executable instructions for determining susceptibility to Bladder Cancer in a human individual, the computer readable medium comprising (i) sequence data identifying at least one allele of at least one polymorphic marker in the individual; and (ii) a routine stored on the computer readable medium and adapted to be executed by a processor to determine risk of developing Bladder Cancer for the at least one polymorphic marker; wherein the at least one polymorphic marker is a marker in the human SLC14A1 gene, or an amino acid substitution in an encoded SLC14A1 protein, that is predictive of susceptibility of Bladder Cancer in humans. In one embodiment, the at least one polymorphic marker is selected from the group consisting of rs1058396, rs11877062, rs2298720 and rs2298719. In another embodiment, the amino acid substitution is a substitution selected from the group consisting of N280D, R4W, K44E and M167V.

In certain embodiments, a report is prepared, which contains results of a determination of susceptibility of bladder cancer. The report may suitably be written in any computer readable medium, printed on paper, or displayed on a visual display.

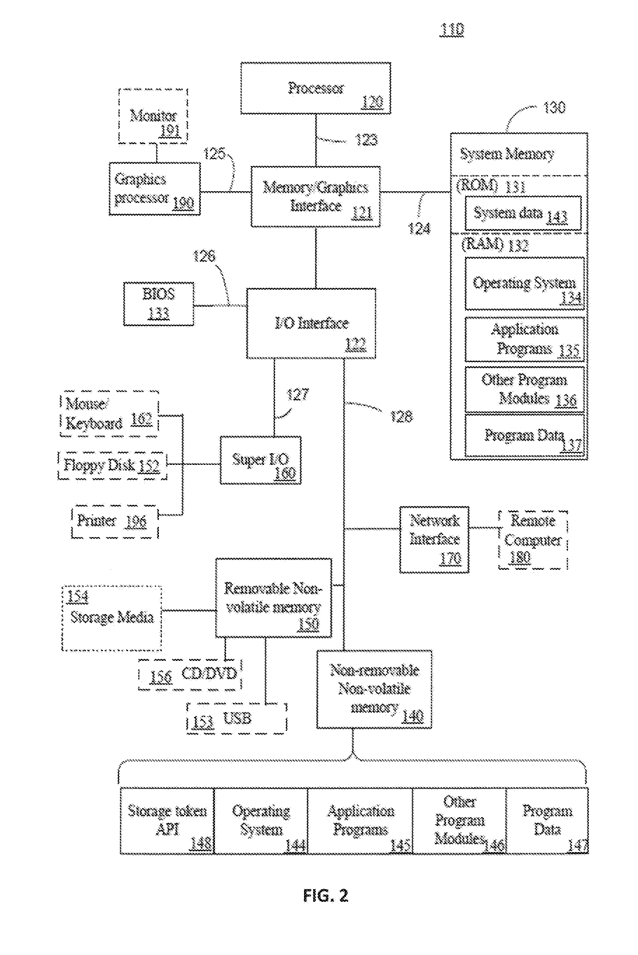

With reference to FIG. 2, a second exemplary system of the invention, which may be used to implement one or more steps of methods of the invention, includes a computing device in the form of a computer 110. Components shown in dashed outline are not technically part of the computer 110, but are used to illustrate the exemplary embodiment of FIG. 2. Components of computer 110 may include, but are not limited to, a processor 120, a system memory 130, a memory/graphics interface 121, also known as a Northbridge chip, and an I/O interface 122, also known as a Southbridge chip. The system memory 130 and a graphics processor 190 may be coupled to the memory/graphics interface 121. A monitor 191 or other graphic output device may be coupled to the graphics processor 190.

A series of system busses may couple various system components including a high speed system bus 123 between the processor 120, the memory/graphics interface 121 and the I/O interface 122, a front-side bus 124 between the memory/graphics interface 121 and the system memory 130, and an advanced graphics processing (AGP) bus 125 between the memory/graphics interface 121 and the graphics processor 190. The system bus 123 may be any of several types of bus structures including, by way of example, and not limitation, such architectures include Industry Standard Architecture (USA) bus, Micro Channel Architecture (MCA) bus and Enhanced ISA (EISA) bus. As system architectures evolve, other bus architectures and chip sets may be used but often generally follow this pattern. For example, companies such as Intel and AMD support the Intel Hub Architecture (IHA) and the Hypertransport™ architecture, respectively.

The computer 110 typically includes a variety of computer-readable media. Computer-readable media can be any available media that can be accessed by computer 110 and includes both volatile and nonvolatile media, removable and non-removable media. By way of example, and not limitation, computer readable media may comprise computer storage media. Computer storage media includes both volatile and nonvolatile, removable and non-removable media implemented in any method or technology for storage of information such as computer readable instructions, data structures, program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other physical medium which can be used to store the desired information and which can accessed by computer 110.

The system memory 130 includes computer storage media in the form of volatile and/or nonvolatile memory such as read only memory (ROM) 131 and random access memory (RAM) 132. The system ROM 131 may contain permanent system data 143, such as identifying and manufacturing information. In some embodiments, a basic input/output system (BIOS) may also be stored in system ROM 131. RAM 132 typically contains data and/or program modules that are immediately accessible to and/or presently being operated on by processor 120. By way of example, and not limitation, FIG. 5 illustrates operating system 134, application programs 135, other program modules 136, and program data 137.

The I/O interface 122 may couple the system bus 123 with a number of other busses 126, 127 and 128 that couple a variety of internal and external devices to the computer 110. A serial peripheral interface (SPI) bus 126 may connect to a basic input/output system (BIOS) memory 133 containing the basic routines that help to transfer information between elements within computer 110, such as during start-up.

A super input/output chip 160 may be used to connect to a number of ‘legacy’ peripherals, such as floppy disk 152, keyboard/mouse 162, and printer 196, as examples. The super I/O chip 160 may be connected to the I/O interface 122 with a bus 127, such as a low pin count (LPC) bus, in some embodiments. Various embodiments of the super I/O chip 160 are widely available in the commercial marketplace.

In one embodiment, bus 128 may be a Peripheral Component Interconnect (PCI) bus, or a variation thereof, may be used to connect higher speed peripherals to the I/O interface 122. A PCI bus may also be known as a Mezzanine bus. Variations of the PCI bus include the Peripheral Component Interconnect-Express (PCI-E) and the Peripheral Component Interconnect—Extended (PCI-X) busses, the former having a serial interface and the latter being a backward compatible parallel interface. In other embodiments, bus 128 may be an advanced technology attachment (ATA) bus, in the form of a serial ATA bus (SATA) or parallel ATA (PATA).

The computer 110 may also include other removable/non-removable, volatile/nonvolatile computer storage media. By way of example only, FIG. 2 illustrates a hard disk drive 140 that reads from or writes to non-removable, nonvolatile magnetic media. The hard disk drive 140 may be a conventional hard disk drive.

Removable media, such as a universal serial bus (USB) memory 153, firewire (IEEE 1394), or CD/DVD drive 156 may be connected to the PCI bus 128 directly or through an interface 150. A storage media 154 may coupled through interface 150. Other removable/non-removable, volatile/nonvolatile computer storage media that can be used in the exemplary operating environment include, but are not limited to, magnetic tape cassettes, flash memory cards, digital versatile disks, digital video tape, solid state RAM, solid state ROM, and the like.

The drives and their associated computer storage media discussed above and illustrated in FIG. 2, provide storage of computer readable instructions, data structures, program modules and other data for the computer 110. In FIG. 2, for example, hard disk drive 140 is illustrated as storing operating system 144, application programs 145, other program modules 146, and program data 147. Note that these components can either be the same as or different from operating system 134, application programs 135, other program modules 136, and program data 137. Operating system 144, application programs 145, other program modules 146, and program data 147 are given different numbers here to illustrate that, at a minimum, they are different copies. A user may enter commands and information into the computer 20 through input devices such as a mouse/keyboard 162 or other input device combination. Other input devices (not shown) may include a microphone, joystick, game pad, satellite dish, scanner, or the like. These and other input devices are often connected to the processor 120 through one of the I/O interface busses, such as the SPI 126, the LPC 127, or the PCI 128, but other busses may be used. In some embodiments, other devices may be coupled to parallel ports, infrared interfaces, game ports, and the like (not depicted), via the super I/O chip 160.

The computer 110 may operate in a networked environment using logical connections to one or more remote computers, such as a remote computer 180 via a network interface controller (NIC) 170. The remote computer 180 may be a personal computer, a server, a router, a network PC, a peer device or other common network node, and typically includes many or all of the elements described above relative to the computer 110. The logical connection between the NIC 170 and the remote computer 180 depicted in FIG. 2 may include a local area network (LAN), a wide area network (WAN), or both, but may also include other networks. Such networking environments are commonplace in offices, enterprise-wide computer networks, intranets, and the Internet. The remote computer 180 may also represent a web server supporting interactive sessions with the computer 110, or in the specific case of location-based applications may be a location server or an application server.

In some embodiments, the network interface may use a modem (not depicted) when a broadband connection is not available or is not used. It will be appreciated that the network connection shown is exemplary and other means of establishing a communications link between the computers may be used.

In some variations, the invention is a system for identifying susceptibility to bladder cancer in a human subject. For example, in one variation, the system includes tools for performing at least one step, preferably two or more steps, and in some aspects all steps of a method of the invention, where the tools are operably linked to each other. Operable linkage describes a linkage through which components can function with each other to perform their purpose.
In some variations, a system of the invention is a system for identifying susceptibility to bladder cancer in a human subject, and comprises:
(a) at least one processor;
(b) at least one computer-readable medium;
(c) a susceptibility database operatively coupled to a computer-readable medium of the system and containing population information correlating the presence or absence of one or more alleles of the human SLC14A1 gene and susceptibility to bladder cancer in a population of humans;
(d) a measurement tool that receives an input about the human subject and generates information from the input about the presence or absence of at least one mutant SLC14A1 allele in the human subject; and
(e) an analysis tool or routine that:
   (i) is operatively coupled to the susceptibility database and the information generated by the measurement tool,
   (ii) is stored on a computer-readable medium of the system,
   (iii) is adapted to be executed on a processor of the system, to compare the information about the human subject with the population information in the susceptibility database and generate a conclusion with respect to susceptibility to the condition for the human subject.

Exemplary processors (processing units) include all variety of microprocessors and other processing units used in computing devices. Exemplary computer-readable media are described above. When two or more components of the system involve a processor or a computer-readable medium, the system generally can be created where a single processor and/or computer readable medium is dedicated to a single component of the system; or where two or more functions share a single processor and/or share a single computer readable medium, such that the system contains as few as one processor and/or one computer readable medium. In some variations, it is advantageous to use multiple processors or media, for example, where it is convenient to have components of the system at different locations. For instance, some components of a system may be located at a testing laboratory dedicated to laboratory or data analysis, whereas other components, including components (optional) for supplying input information or obtaining an output communication, may be located at a medical treatment or counseling facility (e.g., doctor's office, health clinic, HMO, pharmacist, geneticist, hospital) and/or at the home or business of the human subject (patient) for whom the testing service is performed.

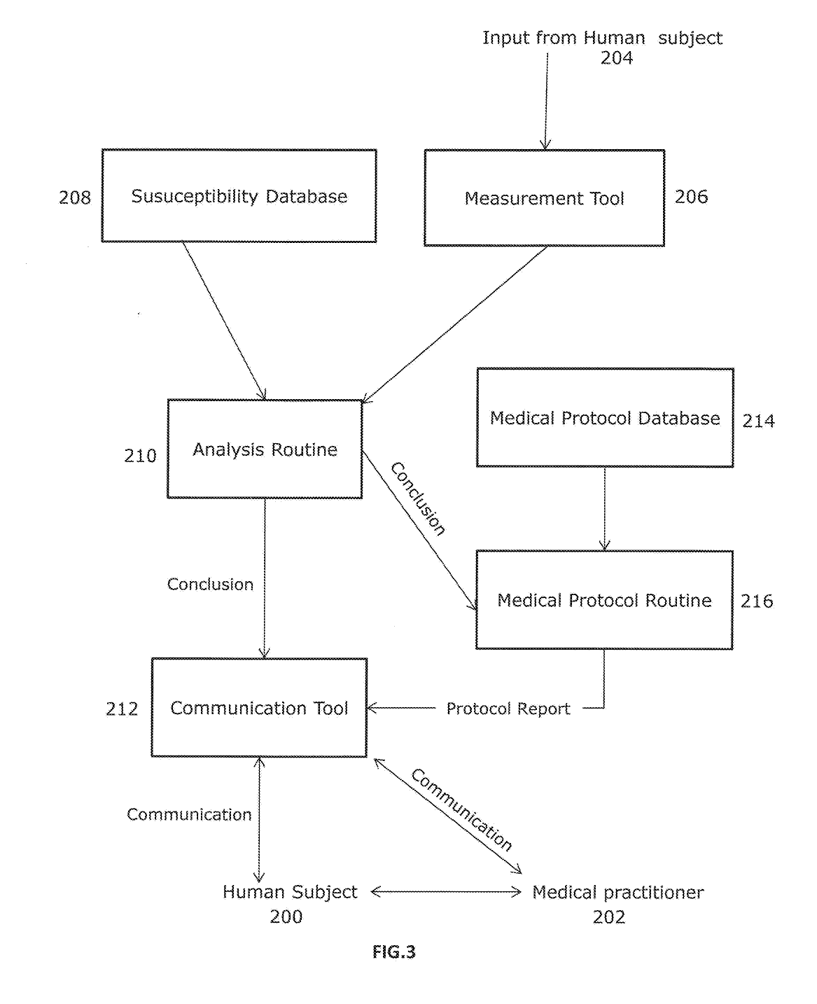

Referring to FIG. 3, an exemplary system includes a susceptibility database 208 that is operatively coupled to a computer-readable medium of the system and that contains population information correlating the presence or absence of one or more alleles of the human SLC14A1 gene and susceptibility to bladder cancer in a population of humans. For example, the one or more alleles of the SLC14A1 gene include mutant alleles that cause, or are indicative of, a SLC14A1 defect such as reduced or lost function, as described elsewhere herein.

In a simple variation, the susceptibility database contains 208 data relating to the frequency that a particular allele of SLC14A1 has been observed in a population of humans with bladder cancer and a population of humans free of bladder cancer. Such data provides an indication as to the relative risk or odds ratio of developing bladder cancer for a human subject that is identified as having the allele in question. In another variation, the susceptibility database includes similar data with respect to two or more alleles of SLC14A1, thereby providing a useful reference if the human subject has any of the two or more alleles. In still another variation, the susceptibility database includes additional quantitative personal, medical, or genetic information about the individuals in the database diagnosed with bladder cancer or free of bladder cancer. Such information includes, but is not limited to, information about parameters such as age, sex, ethnicity, race, medical history, weight, diabetes status, blood pressure, family history of bladder cancer, smoking history, and alcohol use in humans and impact of the at least one parameter on susceptibility to bladder cancer. The information also can include information about other genetic risk factors for bladder cancer besides SLC14A1 alleles. These more robust susceptibility databases can be used by an analysis routine 210 to calculate a combined score with respect to susceptibility or risk for developing bladder cancer.

In addition to the susceptibility database 208, the system further includes a measurement tool 206 programmed to receive an input 204 from or about the human subject and generate an output that contains information about the presence or absence of the at least one SLC14A1 allele of interest. (The input 204 is not part of the system per se but is illustrated in the schematic FIG. 3.) Thus, the input 204 will contain a specimen or contain data from which the presence or absence of the at least one SLC14A1 allele can be directly read, or analytically determined. In a simple variation, the input contains annotated information about genotypes or allele counts for SLC14A1 in the genome of the human subject, in which case no further processing by the measurement tool 206 is required, except possibly transformation of the relevant information about the presence/absence of the SLC14A1 allele into a format compatible for use by the analysis routine 210 of the system.

In another variation, the input 204 from the human subject contains data that is unannotated or insufficiently annotated with respect to SLC14A1, requiring analysis by the measurement tool 206.

For example, the input can be genetic sequence of a chromosomal region or chromosome on which SLC14A1 resides, or whole genome sequence information, or unannotated information from a gene chip analysis of a variable loci in the human subject's genome. In such variations of the invention, the measurement tool 206 comprises a tool, preferably stored on a computer-readable medium of the system and adapted to be executed on a processor of the system, to receive a data input about a subject and determine information about the presence or absence of the at least one mutant SLC14A1 allele in a human subject from the data. For example, the measurement tool 206 contains instructions, preferably executable on a processor of the system, for analyzing the unannotated input data and determining the presence or absence of the SLC14A1 allele of interest in the human subject. Where the input data is genomic sequence information, and the measurement tool optionally comprises a sequence analysis tool stored on a computer readable medium of the system and executable by a processor of the system with instructions for determining the presence or absence of the at least one mutant SLC14A1 allele from the genomic sequence information.

In yet another variation, the input 204 from the human subject comprises a biological sample, such as a fluid (e.g., blood) or tissue sample that contains genetic material that can be analyzed to determine the presence or absence of the SLC14A1 allele of interest. In this variation, an exemplary measurement tool 206 includes laboratory equipment for processing and analyzing the sample to determine the presence or absence (or identity) of the SLC14A1 allele(s) in the human subject. For instance, in one variation, the measurement tool includes: an oligonucleotide microarray (e.g., “gene chip”) containing a plurality of oligonucleotide probes attached to a solid support; a detector for measuring interaction between nucleic acid obtained from or amplified from the biological sample and one or more oligonucleotides on the oligonucleotide microarray to generate detection data; and an analysis tool stored on a computer-readable medium of the system and adapted to be executed on a processor of the system, to determine the presence or absence of the at least one SLC14A1 allele of interest based on the detection data.

To provide another example, in some variations the measurement tool 206 includes: a nucleotide sequencer (e.g., an automated DNA sequencer) that is capable of determining nucleotide sequence information from nucleic acid obtained from or amplified from the biological sample; and an analysis tool stored on a computer-readable medium of the system and adapted to be executed on a processor of the system, to determine the presence or absence of the at least one mutant SLC14A1 allele based on the nucleotide sequence information.

In some variations, the measurement tool 206 further includes additional equipment and/or chemical reagents for processing the biological sample to purify and/or amplify nucleic acid of the human subject for further analysis using a sequencer, gene chip, or other analytical equipment.

The exemplary system further includes an analysis tool or routine 210 that: is operatively coupled to the susceptibility database 208 and operatively coupled to the measurement tool 206, is stored on a computer-readable medium of the system, is adapted to be executed on a processor of the system to compare the information about the human subject with the population information in the susceptibility database 208 and generate a conclusion with respect to susceptibility to bladder cancer for the human subject. In simple terms, the analysis tool 210 looks at the SLC14A1 alleles identified by the measurement tool 206 for the human subject, and compares this information to the susceptibility database 208, to determine a susceptibility to bladder cancer for the subject. The susceptibility can be based on the single parameter (the identity of one or more SLC14A1 alleles), or can involve a calculation based on other genetic and non-genetic data, as described above, that is collected and included as part of the input 204 from the human subject, and that also is stored in the susceptibility database 208 with respect to a population of other humans. Generally speaking, each parameter of interest is weighted to provide a conclusion with respect to susceptibility to bladder cancer. Such a conclusion is expressed in the conclusion in any statistically useful form, for example, as an odds ratio, a relative risk, or a lifetime risk for subject developing the condition.

In some variations of the invention, the system as just described further includes a communication tool 212. For example, the communication tool is operatively connected to the analysis routine 210 and comprises a routine stored on a computer-readable medium of the system and adapted to be executed on a processor of the system, to: generate a communication containing the conclusion; and to transmit the communication to the human subject 200 or the medical practitioner 202, and/or enable the subject or medical practitioner to access the communication. (The subject and medical practitioner are depicted in the schematic FIG. 3, but are not part of the system per se, though they may be considered users of the system. The communication tool 212 provides an interface for communicating to the subject, or to a medical practitioner for the subject (e.g., doctor, nurse, genetic counselor), the conclusion generated by the analysis tool 210 with respect to susceptibility to the condition for the subject. Usually, if the communication is obtained by or delivered to the medical practitioner 202, the medical practitioner will share the communication with the human subject 200 and/or counsel the human subject about the medical significance of the communication. In some variations, the communication is provided in a tangible form, such as a printed report or report stored on a computer readable medium such as a flash drive or optical disk.

In some variations, the communication is provided electronically with an output that is visible on a video display or audio output (e.g., speaker). In some variations, the communication is transmitted to the subject or the medical practitioner, e.g., electronically or through the mail. In some variations, the system is designed to permit the subject or medical practitioner to access the communication, e.g., by telephone or computer. For instance, the system may include software residing on a memory and executed by a processor of a computer used by the human subject or the medical practitioner, with which the subject or practitioner can access the communication, preferably securely, over the internet or other network connection. In some variations of the system, this computer will be located remotely from other components of the system, e.g., at a location of the human subject's or medical practitioner's choosing.

In some variations of the invention, the system as described (including embodiments with or without the communication tool) further includes components that add a treatment or prophylaxis utility to the system. For instance, value is added to a determination of susceptibility to bladder cancer when a medical practitioner can prescribe or administer a standard of care that can reduce susceptibility to bladder cancer; and/or delay onset of bladder cancer; and/or increase the likelihood of detecting the cancer at an early stage. Exemplary lifestyle change protocols include loss of weight, increase in exercise, cessation of unhealthy behaviors such as smoking, and change of diet. Exemplary medicinal and surgical intervention protocols include administration of pharmaceutical agents for prophylaxis; and surgery.

For example, in some variations, the system further includes a medical protocol database 214 operatively connected to a computer-readable medium of the system and containing information correlating the presence or absence of the at least one SLC14A1 allele of interest and medical protocols for human subjects at risk for the cancer. Such medical protocols include any variety of medicines, lifestyle changes, diagnostic tests, increased frequencies of diagnostic tests, and the like that are designed to achieve one of the aforementioned goals. The information correlating a SLC14A1 allele with protocols could include, for example, information about the success with which the cancer is avoided or delayed, or success with which the cancer is detected early and treated, if a subject has a SLC14A1 susceptibility allele and follows a protocol.

The system of this embodiment further includes a medical protocol tool or routine 216, operatively connected to the medical protocol database 214 and to the analysis tool or routine 210. The medical protocol tool or routine 216 preferably is stored on a computer-readable medium of the system, and adapted to be executed on a processor of the system, to: (i) compare (or correlate) the conclusion that is obtained from the analysis routine 210 (with respect to susceptibility to bladder cancer for the subject) and the medical protocol database 214, and (ii) generate a protocol report with respect to the probability that one or more medical protocols in the medical protocol database will achieve one or more of the goals of reducing susceptibility to the cancer; delaying onset of the cancer; and increasing the likelihood of detecting the cancer at an early stage to facilitate early treatment. The probability can be based on empirical evidence collected from a population of humans and expressed either in absolute terms (e.g., compared to making no intervention), or expressed in relative terms, to highlight the comparative or additive benefits of two or more protocols.

Some variations of the system include the communication tool 212. In some examples, the communication tool generates a communication that includes the protocol report in addition to, or instead of, the conclusion with respect to susceptibility.

Information about SLC14A1 allele status not only can provide useful information about identifying or quantifying susceptibility to the cancer; it can also provide useful information about possible causative factors for a human subject identified with the cancer, and useful information about therapies for the patient. In some variations, systems of the invention are useful for these purposes.

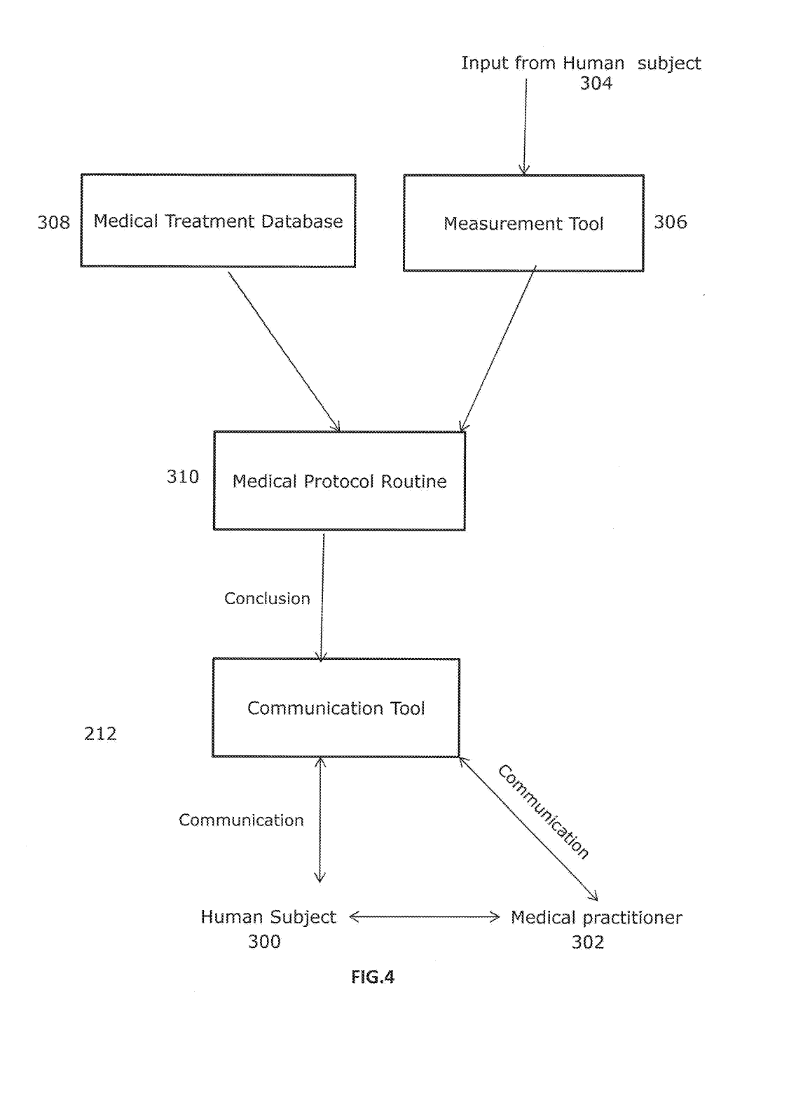

For instance, in some variations the invention is a system for assessing or selecting a treatment protocol for a subject diagnosed with bladder cancer. An exemplary system, schematically depicted in FIG. 4, comprises:
(a) at least one processor;
(b) at least one computer-readable medium;
(c) a medical treatment database 308 operatively connected to a computer-readable medium of the system and containing information correlating the presence or absence of at least one SLC14A1 allele and efficacy of treatment regimens for bladder cancer;
(d) a measurement tool 306 to receive an input (304, depicted in FIG. 4 but not part of the system per se) about the human subject and generate information from the input 304 about the presence or absence of the at least one SLC14A1 allele in a human subject diagnosed with bladder cancer; and
(e) a medical protocol routine or tool 310 operatively coupled to the medical treatment database 308 and the measurement tool 306, stored on a computer-readable medium of the system, and adapted to be executed on a processor of the system, to compare the information with respect to presence or absence of the at least one SLC14A1 allele for the subject and the medical treatment database, and generate a conclusion with respect to at least one of:
   (i) the probability that one or more medical treatments will be efficacious for treatment of bladder cancer for the patient; and
   (ii) which of two or more medical treatments for bladder cancer will be more efficacious for the patient.

Preferably, such a system further includes a communication tool 312 operatively connected to the medical protocol tool or routine 310 for communicating the conclusion to the subject 300, or to a medical practitioner for the subject 302 (both depicted in the schematic of FIG. 4, but not part of the system per se). An exemplary communication tool comprises a routine stored on a computer-readable medium of the system and adapted to be executed on a processor of the system, to generate a communication containing the conclusion; and transmit the communication to the subject or the medical practitioner, or enable the subject or medical practitioner to access the communication.

In a further embodiment, the invention provides a computer-readable medium having computer executable instructions for determining susceptibility to bladder cancer in a human individual, the computer readable medium comprising (i) sequence data identifying at least one allele of at least one polymorphic marker in the individual; and (ii) a routine stored on the computer readable medium and adapted to be executed by a processor to determine risk of developing bladder cancer for the at least one polymorphic marker; wherein the at least one polymorphic marker is a marker in the human SLC14A1 gene, or an amino acid substitution in an encoded SLC14A1 protein, that is predictive of susceptibility of bladder cancer in humans. In one embodiment, the at least one polymorphic marker is selected from the group consisting of rs1058396, rs11877062, rs2298720 and rs2298719. In one preferred embodiment, the polymorphic marker is rs1058396. In another embodiment, the amino acid substitution is selected from the group consisting of an N280D substitution, a R4W substitution, a K44E substitution and a M167V substitution. In one preferred embodiment, the amino acid substitution is a N280D substitution.

In certain embodiments, a report is prepared, which contains results of a determination of susceptibility of bladder cancer. The report may suitably be written in any computer readable medium, printed on paper, or displayed on a visual display.

### Nucleic Acids and Polypeptides

The nucleic acids and polypeptides described herein can be used in methods and kits of the present invention. An “isolated” nucleic acid molecule, as used herein, is one that is separated from nucleic acids that normally flank the gene or nucleotide sequence (as in genomic sequences) and/or has been completely or partially purified from other transcribed sequences (e.g., as in an RNA library). For example, an isolated nucleic acid of the invention can be substantially isolated with respect to the complex cellular milieu in which it naturally occurs, or culture medium when produced by recombinant techniques, or chemical precursors or other chemicals when chemically synthesized. In some instances, the isolated material will form part of a composition (for example, a crude extract containing other substances), buffer system or reagent mix. In other circumstances, the material can be purified to essential homogeneity, for example as determined by polyacrylamide gel electrophoresis (PAGE) or column chromatography (e.g., HPLC). An isolated nucleic acid molecule of the invention can comprise at least about 50%, at least about 80% or at least about 90% (on a molar basis) of all macromolecular species present. With regard to genomic DNA, the term “isolated” also can refer to nucleic acid molecules that are separated from the chromosome with which the genomic DNA is naturally associated. For example, the isolated nucleic acid molecule can contain less than about 250 kb, 200 kb, 150 kb, 100 kb, 75 kb, 50 kb, 25 kb, 10 kb, 5 kb, 4 kb, 3 kb, 2 kb, 1 kb, 0.5 kb or 0.1 kb of the nucleotides that flank the nucleic acid molecule in the genomic DNA of the cell from which the nucleic acid molecule is derived.

The nucleic acid molecule can be fused to other coding or regulatory sequences and still be considered isolated. Thus, recombinant DNA contained in a vector is included in the definition of “isolated” as used herein. Also, isolated nucleic acid molecules include recombinant DNA molecules in heterologous host cells or heterologous organisms, as well as partially or substantially purified DNA molecules in solution. “Isolated” nucleic acid molecules also encompass in vivo and in vitro RNA transcripts of the DNA molecules of the present invention. An isolated nucleic acid molecule or nucleotide sequence can include a nucleic acid molecule or nucleotide sequence that is synthesized chemically or by recombinant means. Such isolated nucleotide sequences are useful, for example, in the manufacture of the encoded polypeptide, as probes for isolating homologous sequences (e.g., from other mammalian species), for gene mapping (e.g., by in situ hybridization with chromosomes), or for detecting expression of the gene in tissue (e.g., human tissue), such as by Northern blot analysis or other hybridization techniques.

The invention also pertains to nucleic acid molecules that hybridize under high stringency hybridization conditions, such as for selective hybridization, to a nucleotide sequence described herein (e.g., nucleic acid molecules that specifically hybridize to a nucleotide sequence containing a polymorphic site associated with a marker or haplotype described herein). Such nucleic acid molecules can be detected and/or isolated by allele- or sequence-specific hybridization (e.g., under high stringency conditions). Stringency conditions and methods for nucleic acid hybridizations are well known to the skilled person (see, e.g., Current Protocols in Molecular Biology, Ausubel, F. et al, John Wiley & Sons, (1998), and Kraus, M. and Aaronson, S., Methods Enzymol., 200:546-556 (1991), the entire teachings of which are incorporated by reference herein.

The percent identity of two nucleotide or amino acid sequences can be determined by aligning the sequences for optimal comparison purposes (e.g., gaps can be introduced in the sequence of a first sequence). The nucleotides or amino acids at corresponding positions are then compared, and the percent identity between the two sequences is a function of the number of identical positions shared by the sequences (i.e., % identity=#of identical positions/total#of positions×100). In certain embodiments, the length of a sequence aligned for comparison purposes is at least 30%, at least 40%, at least 50%, at least 60%, at least 70%, at least 80%, at least 90%, or at least 95%, of the length of the reference sequence. The actual comparison of the two sequences can be accomplished by well-known methods, for example, using a mathematical algorithm. A non-limiting example of such a mathematical algorithm is described in Karlin, S, and Altschul, S., Proc. Natl. Acad. Sci. USA, 90:5873-5877 (1993). Such an algorithm is incorporated into the NBLAST and XBLAST programs (version 2.0), as described in Altschul, S. et al. , Nucleic Acids Res., 25:3389-3402 (1997). When utilizing BLAST and Gapped BLAST programs, the default parameters of the respective programs (e.g., NBLAST) can be used. See the website on the world wide web at ncbi.nlm.nih.gov. In one embodiment, parameters for sequence comparison can be set at score=100, wordlength=12, or can be varied (e.g., W=5 or W=20). Another example of an algorithm is BLAT (Kent, W. J. Genome Res. 12:656-64 (2002)).

Other examples include the algorithm of Myers and Miller, CABIOS (1989), ADVANCE and ADAM as described in Torellis, A. and Robotti, C., Comput. Appl. Biosci. 10:3-5 (1994); and FASTA described in Pearson, W. and Lipman, D., Proc. Natl. Acad. Sci. USA, 85:2444-48 (1988). In another embodiment, the percent identity between two amino acid sequences can be accomplished using the GAP program in the GCG software package (Accelrys, Cambridge, UK).

The present invention also provides isolated nucleic acid molecules that contain a fragment or portion that hybridizes under highly stringent conditions to a nucleic acid that comprises, or consists of, the nucleotide sequence of the human SLC14A1 gene as set forth in SEQ ID NO:134, any one of SEQ ID NO:1-132, or a nucleotide sequence comprising, or consisting of, the complement of the nucleotide sequence of the human SLC14A1 gene as set forth in SEQ ID NO:134 or any one of SEQ ID NO:1-132. The nucleic acid fragments of the invention are at least about 15, at least about 18, 20, 23 or 25 nucleotides, and can be 30, 40, 50, 100, 200, 500, 1000, 10,000 or more nucleotides in length.

The nucleic acid fragments of the invention are used as probes or primers in assays such as those described herein. “Probes” or “primers” are oligonucleotides that hybridize in a base-specific manner to a complementary strand of a nucleic acid molecule. In addition to DNA and RNA, such probes and primers include polypeptide nucleic acids (PNA), as described in Nielsen, P. et al. , Science 254:1497-1500 (1991). A probe or primer comprises a region of nucleotide sequence that hybridizes to at least about 15, typically about 20-25, and in certain embodiments about 40, 50 or 75, consecutive nucleotides of a nucleic acid molecule. In one embodiment, the probe or primer comprises at least one allele of at least one polymorphic marker or at least one haplotype described herein, or the complement thereof. In particular embodiments, a probe or primer can comprise 100 or fewer nucleotides; for example, in certain embodiments from 6 to 50 nucleotides, or, for example, from 12 to 30 nucleotides. In other embodiments, the probe or primer is at least 70% identical, at least 80% identical, at least 85% identical, at least 90% identical, or at least 95% identical, to the contiguous nucleotide sequence or to the complement of the contiguous nucleotide sequence. In another embodiment, the probe or primer is capable of selectively hybridizing to the contiguous nucleotide sequence or to the complement of the contiguous nucleotide sequence. Often, the probe or primer further comprises a label, e.g., a radioisotope, a fluorescent label, an enzyme label, an enzyme co-factor label, a magnetic label, a spin label, an epitope label.

The nucleic acid molecules of the invention, such as those described above, can be identified and isolated using standard molecular biology techniques well known to the skilled person. The amplified DNA can be labeled (e.g., radiolabeled, fluorescently labeled) and used as a probe for screening a cDNA library derived from human cells. The cDNA can be derived from mRNA and contained in a suitable vector. Corresponding clones can be isolated, DNA obtained following in vivo excision, and the cloned insert can be sequenced in either or both orientations by art-recognized methods to identify the correct reading frame encoding a polypeptide of the appropriate molecular weight. Using these or similar methods, the polypeptide and the DNA encoding the polypeptide can be isolated, sequenced and further characterized.

### Antibodies

The invention also provides antibodies which bind to an epitope comprising either a variant amino acid sequence (e.g., comprising an amino acid substitution) encoded by a variant allele or the reference amino acid sequence encoded by the corresponding non-variant or wild-type allele. The term “antibody” as used herein refers to immunoglobulin molecules and immunologically active portions of immunoglobulin molecules, i.e., molecules that contain antigen-binding sites that specifically bind an antigen. A molecule that specifically binds to a polypeptide of the invention is a molecule that binds to that polypeptide or a fragment thereof, but does not substantially bind other molecules in a sample, e.g., a biological sample, which naturally contains the polypeptide. Examples of immunologically active portions of immunoglobulin molecules include F(ab) and F(ab′)₂ fragments which can be generated by treating the antibody with an enzyme such as pepsin. The invention provides polyclonal and monoclonal antibodies that bind to a polypeptide of the invention. The term “monoclonal antibody” or “monoclonal antibody composition”, as used herein, refers to a population of antibody molecules that contain only one species of an antigen binding site capable of immunoreacting with a particular epitope of a polypeptide of the invention. A monoclonal antibody composition thus typically displays a single binding affinity for a particular polypeptide of the invention with which it immunoreacts.

Polyclonal antibodies can be prepared as described above by immunizing a suitable subject with a desired immunogen, e.g., polypeptide of the invention or a fragment thereof. The antibody titer in the immunized subject can be monitored over time by standard techniques, such as with an enzyme linked immunosorbent assay (ELISA) using immobilized polypeptide. If desired, the antibody molecules directed against the polypeptide can be isolated from the mammal (e.g., from the blood) and further purified by well-known techniques, such as protein A chromatography to obtain the IgG fraction. At an appropriate time after immunization, e.g., when the antibody titers are highest, antibody-producing cells can be obtained from the subject and used to prepare monoclonal antibodies by standard techniques, such as the hybridoma technique originally described by Kohler and Milstein, Nature 256:495-497 (1975), the human B cell hybridoma technique (Kozbor et al. , Immunol. Today 4: 72 (1983)), the EBV-hybridoma technique (Cole et al. , Monoclonal Antibodies and Cancer Therapy, Alan R. Liss, 1985, Inc., pp. 77-96) or trioma techniques. The technology for producing hybridomas is well known (see generally Current Protocols in Immunology (1994) Coligan et al., (eds.) John Wiley & Sons, Inc., New York, N.Y.). Briefly, an immortal cell line (typically a myeloma) is fused to lymphocytes (typically splenocytes) from a mammal immunized with an immunogen as described above, and the culture supernatants of the resulting hybridoma cells are screened to identify a hybridoma producing a monoclonal antibody that binds a polypeptide of the invention.

Any of the many well known protocols used for fusing lymphocytes and immortalized cell lines can be applied for the purpose of generating a monoclonal antibody to a polypeptide of the invention (see, e.g., Current Protocols in Immunology, supra; Galfre et al. , Nature 266:55052 (1977); R. H. Kenneth, in Monoclonal Antibodies: A New Dimension In Biological Analyses, Plenum Publishing Corp., New York, N.Y. (1980); and Lerner, Yale J. Biol. Med. 54:387-402 (1981)). Moreover, the ordinarily skilled worker will appreciate that there are many variations of such methods that also would be useful.

Alternative to preparing monoclonal antibody-secreting hybridomas, a monoclonal antibody to a polypeptide of the invention can be identified and isolated by screening a recombinant combinatorial immunoglobulin library (e.g., an antibody phage display library) with the polypeptide to thereby isolate immunoglobulin library members that bind the polypeptide. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. , Bio/Technology 9: 1370-1372 (1991); Hay et al. , Hum. Antibod. Hybridomas 3:81-85 (1992); Huse et al. , Science 246: 1275-1281 (1989); and Griffiths et al. , EMBO J. 12:725-734 (1993).

Additionally, recombinant antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art.

In general, antibodies of the invention (e.g., a monoclonal antibody) can be used to isolate a polypeptide of the invention by standard techniques, such as affinity chromatography or immunoprecipitation. A polypeptide-specific antibody can facilitate the purification of natural polypeptide from cells and of recombinantly produced polypeptide expressed in host cells. Moreover, an antibody specific for a polypeptide of the invention can be used to detect the polypeptide (e.g., in a cellular lysate, cell supernatant, or tissue sample) in order to evaluate the abundance and pattern of expression of the polypeptide. Antibodies can be used diagnostically to monitor protein levels in tissue as part of a clinical testing procedure, e.g., to, for example, determine the efficacy of a given treatment regimen. The antibody can be coupled to a detectable substance to facilitate its detection. Examples of detectable substances include various enzymes, prosthetic groups, fluorescent materials, luminescent materials, bioluminescent materials, and radioactive materials. Examples of suitable enzymes include horseradish peroxidase, alkaline phosphatase, beta-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

Antibodies may also be useful in pharmacogenomic analysis. In such embodiments, antibodies against variant proteins encoded by nucleic acids according to the invention, such as variant proteins that are encoded by nucleic acids that contain at least one polymorpic marker of the invention, can be used to identify individuals that require modified treatment modalities.

Antibodies can furthermore be useful for assessing expression of variant proteins in disease states, such as in active stages of a disease, or in an individual with a predisposition to a disease related to the function of the protein, in particular urinary bladder cancer. Antibodies specific for a variant protein of the present invention can be used to screen for the presence of the variant protein, for example to screen for a predisposition to Bladder Cancer as indicated by the presence of the variant protein.

Antibodies can be used in other methods. Thus, antibodies are useful as diagnostic tools for evaluating proteins, such as variant proteins described herein, in conjunction with analysis by electrophoretic mobility, isoelectric point, tryptic or other protease digest, or for use in other physical assays known to those skilled in the art. Antibodies may also be used in tissue typing. In one such embodiment, a specific variant protein has been correlated with expression in a specific tissue type, and antibodies specific for the variant protein can then be used to identify the specific tissue type.

Subcellular localization of proteins, including variant proteins, can also be determined using antibodies, and can be applied to assess aberrant subcellular localization of the protein in cells in various tissues. Such use can be applied in genetic testing, but also in monitoring a particular treatment modality. In the case where treatment is aimed at correcting the expression level or presence of the variant protein or aberrant tissue distribution or developmental expression of the variant protein, antibodies specific for the variant protein or fragments thereof can be used to monitor therapeutic efficacy.

Antibodies are further useful for inhibiting variant protein function, for example by blocking the binding of a variant protein to a binding molecule or partner. Such uses can also be applied in a therapeutic context in which treatment involves inhibiting a variant protein's function. An antibody can be for example be used to block or competitively inhibit binding, thereby modulating (i.e., agonizing or antagonizing) the activity of the protein. Antibodies can be prepared against specific protein fragments containing sites required for specific function or against an intact protein that is associated with a cell or cell membrane. For administration in vivo, an antibody may be linked with an additional therapeutic payload, such as radionuclide, an enzyme, an immunogenic epitope, or a cytotoxic agent, including bacterial toxins (diphtheria or plant toxins, such as ricin). The in vivo half-life of an antibody or a fragment thereof may be increased by pegylation through conjugation to polyethylene glycol.

The present invention further relates to kits for using antibodies in the methods described herein. This includes, but is not limited to, kits for detecting the presence of a variant protein in a test sample. One preferred embodiment comprises antibodies such as a labelled or labelable antibody and a compound or agent for detecting variant proteins in a biological sample, means for determining the amount or the presence and/or absence of variant protein in the sample, and means for comparing the amount of variant protein in the sample with a standard, as well as instructions for use of the kit.

The present invention will now be exemplified by the following non-limiting example.

### Example 1

Genetic association results on a total of 595 Icelandic Bladder Cancer cases and 37,075 Icelandic controls and close to 1,600 Dutch Bladder Cancer cases and 1,800 Dutch controls were analyzed together. The data analysed included a total of 2.5 million SNPs, including SNPs from the HumanHap 370DUO bead chip and SNPs imputed from the HapMap data. Using this dataset as a starting point, a focused analysis of the association between bladder cancer and about 20,000 non-synonymous SNPs was conducted. Based on study groups from Iceland and the Netherlands, this analysis yielded an association signal on chromosome 18q12.3, with the strongest signal observed for marker rs1058396. This SNP is located in the SLC14A1 gene where it causes an amino acid variation, N280D (D conferring increased risk).

Association of rs1058396 was further confirmed by analysis (Centaurus genotyping) of additional samples from, Belgium, Germany, Eastern Europe, Italy (Brescia), Italy (Torino), Sweden and UK, giving an overall result of OR=0.90 and a p-value of 3.7×10⁻⁵ (Table 3).

In addition to rs1058396, a second non-synonymous SNP in the SLC14A1 gene, rs11877062 (causes amino acid change R4W), showed association with bladder cancer in the combined analysis of the Icelandic and Dutch sample sets (Table 4). The association between rs11877062 and bladder cancer was found to have an OR of 1.14 for the C allele (encoding W at position 4 in the encoded SLC14A1 polypeptide) and a P value of 7.1×10⁻⁴.

Further association data for the missense variants rs2298719 (encoding M167V) and rs2298720 (encoding K44E) are shown in Table 5 (Iceland data only) and Table 6 (Iceland and Holland data).

TABLE 2
Study groups.

| | | | Average | | |
|---|---|---|---|---|---|
| | | | age at | | |
| | | | diagnosis | % males | |
| | # cases | # controls | (range) | (cases) | Study type |
| Discovery groups | | | | | |
| (GWA) | | | | | |
| Iceland | 595 | 37,075 | 68 (20-95) | 76 | Population based |
| Holland discovery | 1,600 | 1,800 | 62 (25-93) | 81 | Population based |
| group | | | | | |
| Total | 2195 | 38,875 | | | |
| Follow up groups | | | | | |
| Belgium, Leuven | 191 | 375 | 68 (40-93) | 86 | Population based |
| Germany, Lutherstadt | 213 | 194 | 65 (20-91) | 86 | Hospital-based |
| Wittenberg | | | | | |
| Eastern Europe | 200 | 526 | 65 (36-90) | 83 | Hospital-based |
| (Hungary, Romania, | | | | | |
| Slovakia) | | | | | |
| Italy, Brescia | 181 | 192 | 63 (22-80) | 100 | Hospital-based |
| Italy, Torino | 323 | 378 | 63 (40-75) | 100 | Hospital-based |
| Sweden, Stockholm | 344 | 1,224 | 69 (32-97) | 67 | Population based |
| UK, Leeds | 724 | 534 | 73 (30-101) | 71 | Hospital-based |
| Total | 2,176 | 3,423 | | | |

TABLE 3
Association between Urinary Bladder Cancer and rs1058396 (N280D in transcript 1
(SEQ ID NO: 209; position 336 in transcript 2 (SEQ ID NO: 133)). OR-values are
reported for the A allele of rs1058396, the risk allele (with OR equalling 1/OR as listed
in the table) is G, encoding an N to D change in the encoded SLC14A1 polypeptide.

| Discovery group | OR | P-value | 95% CI |
|---|---|---|---|
| Iceland chip | 0.84 | 0.0036 | (0.75, 0.94) |
| Holland chip | 0.88 | 0.0034 | (0.81, 0.96) |
| Ice/Hol combined | 0.87 | 4.70E−05 | (0.81, 0.93) |

| | | | | Freq | | Freq |
|---|---|---|---|---|---|---|
| Follow up groups | OR | P-value | # affected | affected | # controls | controls |
| BEL | 1.1475 | 0.285541 | 191 | 0.515707 | 375 | 0.481333 |
| GERMANY | 0.8128 | 0.159806 | 213 | 0.448357 | 194 | 0.5 |
| EASTEUR | 0.8437 | 0.158602 | 200 | 0.4775 | 526 | 0.519962 |
| ITABR | 0.7919 | 0.123846 | 181 | 0.483425 | 192 | 0.541667 |
| ITATO | 1.0043 | 1 | 323 | 0.489164 | 378 | 0.488095 |
| SWE | 0.9372 | 0.484814 | 334 | 0.476048 | 1224 | 0.492239 |
| UK | 0.9638 | 0.655682 | 724 | 0.444061 | 534 | 0.453184 |

| Overall* | OR | P-value | 95% CI |
|---|---|---|---|
| | 0.90 | 3.70E−05 | (0.85, 0.94) |

*all cohorts combined

TABLE 4
Association between Uriary Bladder Cancer and allele T of rs11877062
in discovery cohorts of Icelandic and Dutch and combined groups.
The risk allele C confers a risk of 1/0.88 = 1.14.

| Discovery group | P-value | OR | 95% CI |
|---|---|---|---|
| Iceland chip | 0.0011 | 0.82 | (0.73, 0.92) |
| Holland chip* | 0.097 | 0.92 | |
| Combined | 0.00071 | 0.88 | |

*Using 1200 cases

TABLE 5
Association of missense markers within the SLC14A1 gene on Chr 18 with Urinary Bladder Cancer, based on Icelandic imputed genotypes. The
indicated risk allele is for the OR as shown; thus the C allele of rs11877062 is indicative of increased risk of bladder cancer, while the G allele of e.g.
rs1058396 is indicative of increased risk of bladder cancer (OR value indicated in table is for the other allele of the marker (A), which thus has a risk less
than unity, i.e. it confers decreased risk of bladder cancer.

| | Pos | | | | | | | | | | | Seq |
|---|---|---|---|---|---|---|---|---|---|---|---|---|
| | NCBI | Corr | | Freq | Freq | No of | | | Risk | Other | Coding | ID |
| Marker | B36 | P-value | OR | cases | Ctl | Imp gt | Freq | Info | allele | allele | effect* | NO |
| rs11877062 | 41561244 | 0.000379915 | 1.246563 | 0.546494 | 0.495582 | 38082 | 0.496377 | 0.919451 | C | T | R4W | 130 |
| rs2298720 | 41564413 | 0.204434 | 0.842984 | 0.052479 | 0.061077 | 38082 | 0.060942 | 0.843395 | A | G | E44K | 131 |
| rs2298719 | 41570447 | 0.177335 | 1.927272 | 0.996657 | 0.993917 | 38082 | 0.99396 | 0.529603 | A | G | M/START167V | 132 |
| rs1058396 | 41573517 | 0.00424147 | 0.842842 | 0.467748 | 0.510193 | 38082 | 0.50953 | 0.988212 | A | G | D280N | 4 |

*The positions indicated refer to long transcript 2 as identified in SEQ ID NO: 133 for R4W, while the locations of the other three variants refer to their locations in the shorter transcript set forth in SEQ ID NO: 209. The locations in SEQ ID NO: 133 for those variants is as follows: E44K corresponds to E100K in SEQ ID NO: 133, M167V corresponds to M223V in SEQ ID NO: 133 and D280N corresponds to D336N in SEQ ID NO: 133.

TABLE 6
Association of missense markers within the SLC14A1-gene on Chr 18 with Urinary Bladder Cancer, based on Icelandic and
Dutch imputed genotypes from the 1000 Genomes project (http://www.1000genomes.org).

| | Risk | OR | | P-val | OR | | P-val | OR | | P-val | Seq ID |
|---|---|---|---|---|---|---|---|---|---|---|---|
| Marker | allele | Ice | (95% CI) | Ice | Holl | (95% CI) | Holl | comb | (95% CI) | comb | NO |
| rs1058396 | G | 0.84 | (0.75, 0.94) | 0.0036 | 0.88 | (0.81, 0.96) | 0.0034 | 0.87 | (0.81, 0.93) | 4.70E−05 | 4 |
| rs11877062 | T | 0.82 | (0.73, 0.92) | 0.0011 | 0.9 | (0.83, 0.98) | 0.017 | 0.87 | (0.82, 0.94) | 0.00012 | 130 |
| rs2298720 | A | 0.8 | (0.62, 1.05) | 0.11 | 1.07 | (0.90, 1.27) | 0.42 | 0.99 | (0.85, 1.14) | 0.86 | 131 |

### Methods

The study was approved by the Data Protection Commission of Iceland and the National Bioethics Committee of Iceland. Written informed consent was obtained from all study participants. Personal identifiers associated with medical information and blood samples were encrypted with a third-party encryption system as provided by the Data Protection Commission of Iceland.

### Illumina Genome-Wide Genotyping.

The Icelandic chip typed samples were assayed with the Illumina HumanHap300 or HumanHap CNV370 bead chips at deCODE genetics. The bead chips contained 317,503 and 370,404 haplotype tagging SNPs derived from phase I of the International HapMap project. Only SNPs present on both chips were included in the analysis and SNPs were excluded if they had (i) yield lower than 95% in cases or controls, (ii) minor allele frequency less than 1% in the population, or (iii) showed significant deviation from Hardy-Weinberg equilibrium in the controls (P<0.001). All samples with a call rate below 98% were excluded from the analysis. The final analysis was based on direct genotyping of 289,658 autosomal SNPs.

### Single SNP Genotyping.

Single SNP genotyping was carried out by deCODE genetics applying the Centaurus (Nanogen) platform (Kutyavin, I. V., et al. Nucleic Acids Res 34:e128 (2006)). The quality of each Centaurus SNP assay was evaluated by genotyping each assay on the CEU samples and comparing the results with the HapMap data. All assays had mismatch rate<0.5%. Additionally, all markers were genotyped again on more than 10% of samples typed with the Illumina platform, resulting in an observed mismatch in less than 0.5% of samples.

### Whole-Genome Sequencing.

Sample preparation: Paired-end libraries for sequencing were prepared according to manufacturer's instructions (Illumina). In short, approximately 5 micrograms of genomic DNA, isolated from frozen blood samples, was fragmented to a mean target size of 300 basepairs (bp) using a Covaris E210 instrument. The resulting fragmented DNA was end-repaired using T4 and Klenow polymerases and T4 polynucleotide kinase with 10 mM dNTP's followed by addition of an “A” base at the ends using Klenow exo fragment (3′ to 5′-exo minus) and dATP (1 mM). Sequencing adaptors containing “T” overhangs were ligated to the DNA products followed by agarose (2%) gel electrophoresis. Fragments of about 400 bp were isolated from the gels (Qiagen Gel Extraction Kit) and the adaptor-modified DNA fragments were PCR enriched for 10-cycles using Phusion DNA polymerase (Finnzymes Oy) and PCR primers PE 1.0 and PE 2.0 (Illumina). Enriched libraries were further purified using agarose (2%) gel electrophoresis as described above. The quality and concentration of the libraries was assessed with the Agilent 2100 Bioanalyzer using the DNA 1000 LabChip (Agilent). Barcoded libraries were stored at −20° C. All steps in the workflow were monitored using an in-house laboratory information management system with barcode tracking of all samples and reagents. DNA sequencing: Template DNA fragments were hybridized to the surface of flow cells (Illumina PE flowcell, v4) and amplified to form clusters using the Illumina cBot. In brief, DNA (8-10 μM) was denatured followed by hybridization to grafted adaptors on the flowcell. Isothermal bridge amplification using Phusion polymerase was then followed by linearization of the bridged DNA, denaturation, blocking of 3′-ends and hybridization of the sequencing primer. Sequencing-by-synthesis was performed on Illumina GAIIx instruments equipped with paired-end modules. Paired-end libraries were sequenced using 2×101 cycles of incorporation and imaging with Illumina sequencing kits, v4. Each library/sample was initially run on a single lane for validation followed by further sequencing of lanes with targeted cluster densities of 250-300K/mm². Imaging and analysis of the data was performed using the SCS 2.6 and RTA 1.6 software packages from Illumina, respectively. RTA analysis involved conversion of image data to base-calling in real-time. Alignment: For each lane in the DNA sequencing output, the resulting qseq files were converted into fastq files using an inhouse script. All output from sequencing was converted and the Illumina quality filtering flag was retained in the output. The fastq files were then aligned against Build 36 of the human reference sequence using bwa version 0.5.7 (Li, H. & Durbin, R., Bioinformatics 25:1754-60 (2009)). BAM file generation: SAM file output from the alignment was converted into BAM format using samtools version 0.1.8 (Li, H., et al. Bioinformatics 25:2078-9 (2009) and an inhouse script was used to carry the Illumina quality filter flag over to the BAM file. The BAM files for each sample were then merged into a single BAM file using samtools. Finally, Picard version 1.17 (see http://picard.sourceforge.net/) was used to mark duplicates in the resulting sample BAM files.

### SNP Calling and Genotyping in Whole-Genome Sequencing.

A two step approach was applied to SNP genotyping the whole-genome sequencing data. First, a SNP detection step where sequence positions where at least one individual could be determined to be different from the reference sequence with confidence (quality threshold of 20) based on the SNP calling feature of the pileup tool of samtools (Li, H. & Durbin, R., Bioinformatics 25:1754-60 (2009)). SNPs that were always heterozygous, or always homozygous different from the reference were removed. Second, all positions that were flagged as polymorphic were then genotyped using the pileup tool, but since sequencing depth varies and hence the certainty of genotype calls, genotype likelihoods were calculated rather than deterministic calls.

### Genotype Imputation.

We impute the SNPs identified and genotyped through sequencing into all Icelanders that have been phased using long range phasing using the model used by IMPUTE (Marchini, J. et al. Nat Genet. 39:906-13 (2007)). The genotype data from sequencing can be ambiguous due to low sequencing coverage and is not phased. In order phase the sequencing genotypes an iterative algorithm was applied for each SNP with alleles 0 and 1. Let H be the long range phased haplotypes of the sequenced individuals and follow:
1. For each haplotype h in H, use the hidden Markov model of IMPUTE to calculate λₕₖ for every other k in H, a measure of how likely h is to have the same ancestral source as k.
2. For every h in H initialize the parameter θₕ which specifies how likely the 1 allele of the SNP is to occur on the background of h from the genotype likelihoods obtained from sequencing.

If L₀, L₁ and L₂ are the likelihoods of the genotypes 0, 1 and 2 in the individual that carries h, then set

$${θ}_{h} = \frac{{L}_{2} + \frac{1}{2} {L}_{1}}{{L}_{2} + {L}_{1} + {L}_{0}} .$$

3. For every pair of haplotypes h and k in H that are carried by the same individual use the other haplotypes in H to predict thep genotype of the SNP on the backgrounds of h and k:

$${τ}_{h} = {\sum }_{l ∈ H \backslash \{ h \}} {γ}_{h , l} {θ}_{l} and {τ}_{k} = {\sum }_{l ∈ H \backslash \{ k \}} {γ}_{k , l} {θ}_{l} .$$

Combining these predictions with the genotype likelihoods from sequencing gives un-normalized updated phased genotype probabilities:

$${P}_{00} = ( 1 - {τ}_{h} ) ( 1 - {τ}_{k} ) {L}_{0} , \\ {P}_{01} = ( 1 - {τ}_{h} ) {τ}_{k } \frac{1}{2} {L}_{1} , \\ {P}_{10} = {τ}_{h} ( 1 - {τ}_{k} ) \frac{1}{2} {L}_{1} and {P}_{11} = {τ}_{h} {τ}_{k} {L}_{2} .$$

Now use these values to update θₕ and θₖ to

$$\frac{{P}_{10} + {P}_{11}}{{P}_{00} + {P}_{01} + {P}_{10} + {P}_{11}} and \frac{{P}_{01} + {P}_{11}}{{P}_{00} + {P}_{01} + {P}_{10} + {P}_{11}} ,$$

respectively.

4. Repeat step 3 while the maximum difference between iterations is greater than ε. We used ε=10⁻⁷.

Given the long range phased haplotypes and θ the allele of the SNP on a new haplotype h, not in H, is imputed as

$${\sum }_{l ∈ H} {γ}_{h , l} {θ}_{l} .$$

The above algorithm can easily be extended to handle simple family structures such as parent offspring pairs and triads by letting the P distribution run over all founder haplotypes in the family structure. The algorithm also extends trivially to the X-chromosome. If source genotype data is only ambiguous in phase, such as chip genotype data, then the algorithm is still applied but all but one of the Ls will be 0.

### Association Testing.

Logistic regression was used to test for association between SNPs and disease, treating disease status as the response and expected genotype counts from imputation or allele counts from direct genotyping as covariates. Testing was performed using the likelihood ratio statistic.

### Subjects:

### Icelandic Study Population.

Records of all urinary bladder cancer diagnoses were obtained from the Icelandic Cancer Registry (ICR) (http://www.krabbameinsskra.is). The ICR contains all cancer diagnoses in Iceland from Jan. 1, 1955. The ICR contained records of 1,845 Icelandic Bladder Cancer patients diagnosed until Dec. 31, 2009, and all prevalent cases were eligible to participate. The mean participation rate for newly diagnosed cases was 65%. Patients were recruited by trained nurses on behalf of the patients' treating physicians, through special recruitment clinics. Participants in the study donated a blood sample and answered a lifestyle questionnaire. A total of 611 patients (76% males; diagnosed from December 1974 to December 2008) were included in a genome-wide SNP genotyping effort, using the Infinium II assay method and either the Sentrix HumanHap 300 or HumanCNV370-duo BeadChip (Illumina). The median age at diagnosis for all consenting cases was 68 years (range 22-95 years) as compared to 68.5 years for all Bladder Cancer patients in the ICR. The 37,478 controls (41% males; mean age 61 years; SD=21) used in this study consisted of individuals from other ongoing genome-wide association studies at deCODE and represent over 15% of the adult population of Iceland. No individual disease group is represented by more than 10% of the total control group. Cancer patients (prostate, breast, colorectal and lung) were analyzed separately, and the frequency of the sequence variants studied did not differ from other controls. The study was approved by the Data Protection Authority of Iceland and the National Bioethics Committee. Written informed consent was obtained from all patients, relatives and controls. Personal identifiers associated with medical information and blood samples were encrypted with a third-party encryption system in which the Data Protection Authority maintains the code.

### Dutch Population

### The Netherlands, Discovery Population.

The Dutch patients were recruited for the Nijmegen Bladder Cancer Study (see http://dceg.cancer.gov/icbc/membership.html). The Nijmegen Bladder Cancer Study identified patients through the population-based regional cancer registry held by the Comprehensive Cancer Centre East, Nijmegen that serves a region of 1.3 million inhabitants in the eastern part of the Netherlands (www.ikcnet.nl). Patients diagnosed between 1995 and 2009 under the age of 75 years were selected and their vital status and current addresses updated through the hospital information systems of the 7 community hospitals and one university hospital (Radboud University Nijmegen Medical Centre, RUNMC) that are covered by the cancer registry. All patients were invited to the study by the Comprehensive Cancer Center on behalf of the patients' treating physicians. In case of consent, patients were sent a lifestyle questionnaire to fill out and blood samples were collected by Thrombosis Service centers which hold offices in all the communities in the region. The number of participating patients was increased with a non-overlapping series of 376 bladder cancer patients who were recruited previously for a study on gene-environment interactions in three hospitals (RUNMC, Canisius Wilhelmina Hospital, Nijmegen, and Streekziekenhuis Midden-Twente, Hengelo, the Netherlands). All the patients that were selected for the analyses were of self-reported European descent. The median age at diagnosis was 62 (range 25-93) years. 82% of the participants were males. Data on tumor stage and grade were obtained through the cancer registry. The 1,832 control individuals (46% males) were cancer free and frequency-matched for age with the cases. They were recruited within a project entitled “Nijmegen Biomedical Study” (NBS). The details of this study were reported previously (Wetzels, I F., et al. Kidney Int 72:632-7 (2007)). Briefly, this is a population-based survey conducted by the Department of Epidemiology and Biostatistics and the Department of Clinical Chemistry of the Radboud University Nijmegen Medical Centre (RUNMC), in which 9,371 individuals participated from a total of 22,500, age and sex stratified, randomly selected inhabitants of Nijmegen. Control individuals from the Nijmegen Biomedical Study were invited to participate in a study on gene-environment interactions in multifactorial diseases, such as cancer. All the 1,832 participants in the present study are of self-reported European descent and were fully informed about the goals and the procedures of the study. The study protocols of the Nijmegen Bladder Cancer Study and the Nijmegen Biomedical Study were approved by the Institutional Review Board of the RUNMC and all study subjects gave written informed consent.

### Leeds Bladder Cancer Study, United Kingdom.

Details of the Leeds Bladder Cancer Study have been reported previously (Sak, S. C., et al. Br J Cancer 92:2262-5 (2005)). In brief, patients from the urology department of St James's University Hospital, Leeds were recruited from August 2002 to March 2006. All those patients attending for cystoscopy or transurethral resection of a bladder tumor (TURBT) who had previously been found, or were subsequently shown, to have urothelial cell carcinoma of the bladder were included. Exclusion criteria were significant mental impairment or a blood transfusion in the past month. All non-Caucasians were excluded from the study leaving 764 patients. The median age at diagnosis of the patients was 73 years (range 30-101). 71% of the patients were male and 36% of all the patients had a low risk tumor (pTaG1/2). The controls were recruited from the otolaryngology outpatients and ophthalmology inpatient and outpatient departments at St James's Hospital, Leeds, from August 2002 to March 2006. All controls of appropriate age for frequency matching with the cases were approached and recruited if they gave their informed consent. As for the cases, exclusion criteria for the controls were significant mental impairment or a blood transfusion in the past month. Also, controls were excluded if they had symptoms suggestive of bladder cancer, such as haematuria. 2.8% of the controls were non-Caucasian leaving 530 Caucasian controls for the study. 71% of the controls were male. Data were collected by a health questionnaire on smoking habits and smoking history (non- ex- or current smoker, smoking dose in pack-years), occupational exposure history (to plastics, rubber, laboratories, printing, dyes and paints, diesel fumes), family history of bladder cancer, ethnicity and place of birth, and places of birth of parents. The response rate of cases was approximately 99%, that among the controls approximately 80%. Ethical approval for the study was obtained from Leeds (East) Local Research Ethics Committee, project number 02/192.

### Torino Bladder Cancer Case Control Study, Italy.

The source of cases for the Torino bladder cancer study are two urology departments of the main hospital in Torino, the San Giovanni Battista Hospital (Matullo, G., et al. Cancer Epidemiol Biomarkers Prev 14:2569-78 (2005)). Cases are all Caucasian men, aged 40 to 75 years (median 63 years) and living in the Torino metropolitan area. They were newly diagnosed between 1994 and 2006 with a histologically confirmed, invasive or in situ, bladder cancer. Of all the patients with information on stage and grade, 56% were low risk (pTaG1/2). The sources of controls are urology, medical and surgical departments of the same hospital in Torino. All controls are Caucasian men resident in the Torino metropolitan area. They were diagnosed and treated between 1994 and 2006 for benign diseases (such as prostatic hyperplasia, cystitis, hernias, heart failure, asthma, and benign ear diseases). Controls with cancer, liver or renal diseases and smoking related conditions were excluded. The median age of the controls was 57 years (range 40 to 74). Data were collected by a professional interviewer who used a structured questionnaire to interview both cases and controls face-to-face. Data collected included demographics (age, sex, ethnicity, region and education) and smoking. For cases, additional data were collected on tumor histology, tumor site, size, stage, grade, and treatment of the primary tumor. The response rates were 90% for cases and 75% for controls resulting in 328 cases and 389 controls. Ethical approval for the study was obtained from Comitato Etico Interaziendale, A.O.U. San Giovanni Batista—A.). C.T.O./ Maria Adelaide.

### The Brescia Bladder Cancer Study, Italy.

The Brescia bladder cancer study is a hospital-based case-control study. The study was reported in detail previously (Shen, M. et al. , Cancer Epidemiol Biomarkers Prey 12:1234-40 (2003)). In short, the catchment area of the cases and controls was the Province of Brescia, a highly industrialized area in Northern Italy (mainly metal and mechanical industry, construction, transport, textiles) but also with relevant agricultural areas. Cases and controls were enrolled in 1997 to 2000 from the two main city hospitals. The total number of eligible subjects was 216 cases and 220 controls. The response rate (enrolled/eligible) was 93% (N=201) for cases and 97% (N=214) for controls. Only males were included. All cases and controls had Italian nationality and were of Caucasian ethnicity. All cases had to be residents of the Province of Brescia, aged between 20 and 80, and newly diagnosed with histologically confirmed bladder cancer. The median age of the patients was 63 years (range 22-80). 29% of all the patients with known stage and grade had a low risk tumor (pTaG1/2). Controls were patients admitted for various urological non-neoplastic diseases and were frequency matched to cases on age, hospital and period of admission. The study was formally approved by the ethical committee of the hospital where the majority of subjects were recruited. A written informed consent was obtained from all participants. Data were collected from clinical charts (tumor histology, site, grade, stage, treatments, etc.) and by means of face-to-face interviews during hospital admission, using a standardized semi-structured questionnaire. The questionnaire included data on demographics (age, ethnicity, region, education, residence, etc.), and smoking. ISCO and ISIC codes and expert assessments were used for occupational coding. Blood samples were collected from cases and controls for genotyping and DNA adducts analyses.

### The Belgian Case Control Study of Bladder Cancer.

The Belgian study has been reported in detail (Kellen, E., et al. Int J Cancer 118:2572-8 (2006)). In brief, cases were selected from the Limburg Cancer Registry (LIKAR) and were approached through urologists and general practitioners. All cases were diagnosed with histologically confirmed urothelial cell carcinoma of the bladder between 1999 and 2004, and were Caucasian inhabitants of the Belgian province of Limburg. The median age of the patients was 68 years. 86% of all the patients were males. For the recruitment of controls, a request was made to the “Kruispuntbank” of the social security for simple random sampling, stratified by municipality and socio-economic status, among all citizens above 50 years of age of the province. The median age of the controls was 64 years; 59% of the controls were males. Three trained interviewers visited cases and controls at home. Information was collected through a structured interview and a standardized food frequency questionnaire. In addition, biological samples were collected. Data collected included medical history, lifetime smoking history, family history of bladder cancer and a lifetime occupational history. Informed consent was obtained from all participants and the study was approved by the ethical review board of the Medical School of the Catholic University of Leuven, Belgium.

### The Eastern Europe Study Population.

The details of this study have been described previously⁸. Cases and controls were recruited as part of a study designed to evaluate the risk of various cancers due to environmental arsenic exposure in Hungary, Romania and Slovakia between 2002 and 2004. The recruitment was carried out in the counties of Bacs, Bekes, Csongrad and Jasz-Nagykun-Szolnok in Hungary; Bihor and Arad in Romania; and Banska Bystrica and Nitra in Slovakia. The cases (N=214) and controls (N=533) selected were of Hungarian, Romanian and Slovak nationalities. Bladder cancer patients were invited on the basis of histopathological examinations by pathologists. Hospital-based controls were included in the study, subject to fulfillment of a set of criteria. All general hospitals in the study areas were involved in the process of control recruitment. The controls were frequency matched with cases for age, gender, country of residence and ethnicity. Controls included general surgery, orthopedic and trauma patients aged 30-79 years. Patients with malignant tumors, diabetes and cardiovascular diseases were excluded as controls. The median age for the bladder cancer patients was 65 years (range 36-90). 83% of the patients were males. The median age for the controls was 61 years (range 28-83). 51% of the controls were males. The response rates among cases and controls were ˜70%. Of all the patients with known stage and grade information, 28% had a low risk tumor (pTaG1/2). Clinicians took venous blood and other biological samples from cases and controls after consent forms had been signed. Cases and controls recruited to the study were interviewed by trained personnel and completed a general lifestyle questionnaire. Ethnic background for cases and controls was recorded along with other characteristics of the study population. Local ethical boards approved the study plan and design.

### The Swedish Bladder Cancer Study.

The Swedish patients come from a population-based study of urinary bladder cancer patients diagnosed in the Stockholm region in 1995-1996 (Larsson, P. et al. Scand J Urol Nephrol 37:195-201 (2003)). Blood samples from 352 patients were available out of a collection of 538 patients with primary urothelial carcinoma of the bladder. The average age at onset for these patients is 69 years (range 32-97 years) and 67% of the patients are males. Clinical data, including age at onset, grade and stage of tumor, were prospectively obtained from hospitals and urology units in the region. The control samples came from blood donors in the Stockholm region and were from cancer free individuals of both genders. The regional ethical committee approved of the study and all participants gave informed consent.

### Lutherstadt Wittenberg Bladder Cancer Study, Germany.

Details of the bladder cancer cases of this study have been reported previously (Golka, K. et al. , J Toxicol Environ Helath A 71:881-6 (2008)); Golka, K. et al. , Pharmacogenet Genomics (2009)). In brief, 221 patients with a confirmed bladder cancer from the Department of Urology, Paul Gerhardt Foundation, Lutherstadt Wittenberg, Germany, were included. Patients were enrolled from December 1995 to January 1999. Exclusion criterion was a missing written informed consent into the study. The median age of the patients was 65 years (range 20-91); 86% of the patients were males. A total of 214 controls were from the same department of urology, but were admitted for treatment of benign urological diseases. Exclusion criteria were malignant disease in the medical history or a missing written informed consent. The median age of the controls was 68 years (range 29-91); 84% of the controls were males. Data were collected from July 2000 to May 2005. All cases and controls were Caucasians, which was confirmed by questionnaire-based documentation of nationality. Cases and controls were matched for age. Data collected in cases and controls include age, gender, a complete documentation of occupational activities performed at least for 6 months, documentation of work places with known bladder cancer risk over the entire working life, exposures to known or suspected occupational bladder carcinogens, lifetime smoking habits, family history of bladder cancer, numbers of urinary infections treated by drugs during the previous 10 years, place of birth and places of residency for more than 10 years. For bladder cancer cases, data on tumor staging, grading and treatment were taken from the records. First diagnosis of bladder cancer was recorded from July 1979 to January 1999. The local ethics committee approved the study plan and design.

1. A method of determining a susceptibility to Bladder Cancer, the method comprising:
analyzing nucleic acid sequence data from a human individual for at least one polymorphic marker in the human SLC14A1 gene;
wherein different alleles of the at least one polymorphic marker are associated with different susceptibilities to Bladder Cancer in humans, and
determining a susceptibility to Bladder Cancer from the nucleic acid sequence data.

2. The method of claim 1, wherein the nucleic acid sequence data is obtained from a biological sample containing nucleic acid from the human individual.

3. The method of claim 2, wherein the nucleic acid sequence data is obtained using a method that comprises at least one procedure selected from:
(i) amplification of nucleic acid from the biological sample;
(ii) hybridization assay using a nucleic acid probe and nucleic acid from the biological sample;
(iii) hybridization assay using a nucleic acid probe and nucleic acid obtained by amplification of the biological sample, and
(iv) high-throughput sequencing.

4. The method of claim 1, wherein the nucleic acid sequence data is obtained from a preexisting record.

5. The method of claim 4, wherein the preexisting record comprises a genotype dataset.

6. The method of any one of the preceding claims, wherein the analyzing comprises determining the presence or absence of at least one at-risk allele for Bladder Cancer of the polymorphic marker.

7. The method of any one of the preceding claims, wherein the determining comprises comparing the sequence data to a database containing correlation data between the at least one polymorphic marker and susceptibility to Bladder Cancer.

8. The method of any one of the preceding claims, wherein the at least one polymorphic marker encodes a missense substitution, a nonsense substitution, or a truncation in a SLC14A1 protein with sequence as set forth in SEQ ID NO:133.

9. The method of any one of the preceding claims, wherein the at least one polymorphic marker encodes a defective SLC14A1 protein with impaired function selected from the group consisting of: an impaired JK antigen function, and an impaired urea binding function.

10. The method of any one of the preceding claims, wherein the at least one polymorphic marker in the SLC14A1 gene is selected from the group consisting of rs1058396, rs11877062, rs2298720 and rs2298719, and markers in linkage disequilibrium therewith.

11. The method of claim 10, wherein marker in linkage disequilibrium with rs1058396 are selected from the group consisting of the markers set forth in Table 1.

12. The method of claim 6, wherein the at least one at-risk allele is selected from the group consisting of the G allele of marker rs1058936, the C allele of marker rs11877062, the G allele of marker rs2298720, and the A allele of marker rs2298719.

13. A method of determining whether an individual is at increased risk of developing bladder cancer, the method comprising steps of
obtaining a biological sample containing nucleic acid from the individual;
determining, in the biological sample, nucleic acid sequence about the SLC14A1 gene; and
comparing the sequence information to the wild-type nucleic acid sequence of SLC14A1 (SEQ ID NO:134);
wherein an identification of a mutation in SLC14A1 in the individual is indicative that the individual is at increased risk of developing bladder cancer.

14. The method of claim 13, wherein the mutation is a missense mutation, a nonsense mutation, a splice site mutation or a frameshift mutation in SLC14A1.

15. The method of claim 14, wherein the mutation is selected from the group consisting of rs1058396, rs11877062, rs2298720 and rs2298719.

16. A method of determining a susceptibility to Bladder Cancer, the method comprising:
obtaining amino acid sequence data about at least one encoded SLC14A1 protein in a human individual; and
analyzing the amino acid sequence data to determine whether at least one amino acid substitution predictive of increased susceptibility of Bladder Cancer is present;
wherein a determination of the presence of the at least one amino acid substitution is indicative of increased susceptibility of Bladder Cancer for the individual, and wherein a determination of the absence of the at least one amino acid substitution is indicative of the individual not having the increased susceptibility.

17. The method of claim 16, wherein the amino acid sequence data is obtained from a biological sample containing SLC14A1 protein from the human individual.

18. The method of claim 17, wherein the amino acid sequence data is obtained using a method that comprises at least one procedure selected from:
(i) an antibody assay; and
(iii) protein sequencing.

19. The method of claim 16, wherein the amino acid sequence data is obtained from a preexisting record.

20. The method of any one of the claims 16 to 19, wherein the presence of an amino acid selected from the group consisting of:
Aspartic acid at position 336;
Tryptophan at position 4;
Glutamic acid at position 100; and
Valine at position 223;
in an SLC14A1 protein with sequence as set forth in SEQ ID NO:133 is indicative of an increased risk of bladder cancer for the human individual.

21. The method of any one of the preceding claims, further comprising a step of preparing a report containing results from the determination, wherein said report is written in a computer readable medium, printed on paper, or displayed on a visual display.

22. The method of any one of the previous claims, further comprising reporting the susceptibility to at least one entity selected from the group consisting of the individual, a guardian of the individual, a genetic service provider, a physician, a medical organization, and a medical insurer.

23. A method of identification of a marker for use in assessing susceptibility to Bladder Cancer in human individuals, the method comprising
a. identifying at least one polymorphic marker in the human SLC14A1 gene;
b. obtaining sequence information about the at least one polymorphic marker in a group of individuals diagnosed with Bladder Cancer; and
c. obtaining sequence information about the at least one polymorphic marker in a group of control individuals;
wherein determination of a significant difference in frequency of at least one allele in the at least one polymorphism in individuals diagnosed with Bladder Cancer as compared with the frequency of the at least one allele in the control group is indicative of the at least one polymorphism being useful for assessing susceptibility to Bladder Cancer.

24. The method of claim 23, wherein an increase in frequency of the at least one allele in the at least one polymorphism in individuals diagnosed with Bladder Cancer, as compared with the frequency of the at least one allele in the control group, is indicative of the at least one polymorphism being useful for assessing increased susceptibility to Bladder Cancer; and wherein a decrease in frequency of the at least one allele in the at least one polymorphism in individuals diagnosed with Bladder Cancer, as compared with the frequency of the at least one allele in the control group, is indicative of the at least one polymorphism being useful for assessing decreased susceptibility to, or protection against, Bladder Cancer.

25. A method of predicting prognosis of an individual diagnosed with Bladder Cancer, the method comprising
obtaining sequence data about a human individual about at least one polymorphic marker in the human SLC14A1 gene, wherein different alleles of the at least one polymorphic marker are associated with different susceptibilities to Bladder Cancer in humans, and
predicting prognosis of Bladder Cancer from the sequence data.

26. A method of assessing probability of response of a human individual to a therapeutic measure for preventing, treating and/or ameliorating symptoms associated with Bladder Cancer, comprising:
obtaining sequence data about a human individual identifying at least one allele of at least one polymorphic marker in the human SLC14A1 gene, wherein different alleles of the at least one polymorphic marker are associated with different probabilities of response to the therapeutic agent in humans, and
determining the probability of a positive response to the therapeutic agent from the sequence data.

27. The method of claim 26, wherein the therapeutic measure is selected from the group consisting of radiation therapy, chemotherapy and a surgical procedure.

28. A kit for assessing susceptibility to Bladder Cancer in human individuals, the kit comprising:
reagents for selectively detecting at least one risk variant for Bladder Cancer in the individual, wherein the at least one risk variant is a marker in the human SLC14A1 gene or an amino acid marker in an encoded SLC14A1 protein, and
a collection of data comprising correlation data between the at least one at-risk variant and susceptibility to Bladder Cancer.

29. The kit of claim 28, wherein the collection of data is on a computer-readable medium.

30. The kit of claim 28 or claim 20, wherein the at least one at-risk variant in the human SLC14A1 gene is a marker selected from the group consisting of rs1058396, and markers in linkage disequilibrium therewith.

31. The kit of any one of the claims 28 to 30, wherein the kit comprises reagents for detecting no more than 100 alleles in the genome of the individual.

32. The kit of claim 31, wherein the kit comprises reagents for detecting no more than 20 alleles in the genome of the individual.

33. The kit of claim 28, wherein the amino acid variant in an encoded SLC14A1 protein is a variation in a protein with sequence as set forth in SEQ ID NO:133, selected from the group consisting of:
an arginine to tryptophan variation at position 4;
an lysine to glutamic acid variation at position 100;
a methionine to valine variation at position 223; and
an asparagine to aspartic acid variation at position 336.

34. The kit of claim 28 or claim 33, wherein the reagents comprises at least one antibody for selectively detecting the at least one amino acid variant.

35. Use of an oligonucleotide probe in the manufacture of a diagnostic reagent for diagnosing and/or assessing a susceptibility to Bladder Cancer, wherein the probe is capable of hybridizing to a segment of the human SLC14A1 gene with sequence as given by SEQ ID NO:134, and wherein the segment is 15-400 nucleotides in length.

36. The use of claim 35, wherein the segment of the nucleic acid to which the probe is capable of hybridizing comprises a polymorphic site.

37. The use of claim 36, wherein the polymorphic site is selected from the group consisting of rs1058396, and markers in linkage disequilibrium therewith.

38. A computer-readable medium having computer executable instructions for determining susceptibility to Bladder Cancer in a human individual, the computer readable medium comprising:
sequence data identifying at least one allele of at least one polymorphic marker in the individual;
a routine stored on the computer readable medium and adapted to be executed by a processor to determine risk of developing Bladder Cancer for the at least one polymorphic marker;
wherein the at least one polymorphic marker is a marker in the human SLC14A1 gene, or an amino acid variant in an encoded SLC14A1 protein, that is predictive of susceptibility of Bladder Cancer in humans.

39. The computer-readable medium of claim 38, wherein the medium contains data indicative of at least two polymorphic markers.

40. The computer-readable medium of claim 38 or claim 39, wherein the marker in the human SLC14A1 gene is selected from the group consisting of rs1058396, and markers in linkage disequilibrium therewith.

41. The computer-readable medium of claim 38, wherein the amino acid variant is a variant in an encoded SLC14A1 protein with sequence as set forth in SEQ ID NO:133, selected from the group consisting of:
an arginine to tryptophan variation at position 4;
a lysine to glutamic acid variation at position 100;
a methionine to valine variation at position 223; and
an asparagine to aspartic acid variation at position 336.

42. An apparatus for determining a susceptibility to Bladder Cancer in a human individual, comprising:
a processor;
a computer readable memory having computer executable instructions adapted to be executed on the processor to analyze information for at least one human individual with respect to at least one marker in the human SLC14A1 gene that is predictive of susceptibility to Bladder Cancer in humans, or at least one amino acid variation in an encoded SLC14A1 protein, and generate an output based on the marker or amino acid information, wherein the output comprises at least one measure of susceptibility to Bladder Cancer for the human individual.

43. The apparatus of claim 42, wherein the marker information comprises nucleic acid sequence data identifying at least one allele of the at least one marker in the genome of the individual.

44. The apparatus of claim 42, wherein the sequence data comprises a genotype dataset.

45. The apparatus according to claim 42, wherein the computer readable memory further comprises data indicative of the risk of developing Bladder Cancer associated with at least one allele of at least one polymorphic marker, and wherein a risk measure for the human individual is based on a comparison of the marker information for the human individual to the risk of Bladder Cancer associated with the at least one allele of the at least one polymorphic marker.

46. The apparatus according to any one of claims 42-45, wherein the at least one marker is selected from the group consisting of rs1058396, and markers in linkage disequilibrium therewith.

47. The apparatus of claim 42, wherein the amino acid variation is a variation in a protein with sequence as set forth in SEQ ID NO:133, selected from the group consisting of:
an arginine to tryptophan variation at position 4;
a lysine to glutamic acid variation at position 100;
a methionine to valine variation at position 223; and
an asparagine to aspartic acid variation at position 336.

48. A system for identifying susceptibility to bladder cancer in a human subject, the system comprising:
at least one processor;
at least one computer-readable medium;
a susceptibility database operatively coupled to a computer-readable medium of the system and containing population information correlating the presence or absence of one or more alleles of the human SLC14A1 gene and susceptibility to bladder cancer in a population of humans;
a measurement tool that receives an input about the human subject and generates information from the input about the presence or absence of the at least one allele in the human subject; and
an analysis tool that:
is operatively coupled to the susceptibility database and the measurement tool,
is stored on a computer-readable medium of the system,
is adapted to be executed on a processor of the system, to compare the information about the human subject with the population information in the susceptibility database and generate a conclusion with respect to susceptibility to bladder cancer for the human subject.

49. The system according to claims 48, further including:
a communication tool operatively coupled to the analysis tool, stored on a computer-readable medium of the system and adapted to be executed on a processor of the system to communicate to the subject, or to a medical practitioner for the subject, the conclusion with respect to susceptibility to bladder cancer for the subject.

50. The system according to claim 48 or claim 49, wherein the at least one allele is indicative of a SLC14A1 defect selected from the group consisting of a missense substitution, a nonsense substitution or a truncation in a SLC14A1 protein with sequence as set forth in SEQ ID NO:133; and wherein the at least one allele is associated with increased susceptibility to bladder cancer.

51. The system according to claim 50, wherein the at least one allele is indicative of an amino acid substitution in a protein with sequence as set forth in SEQ ID NO:133, selected from the group consisting of:
an arginine to tryptophan substitution at position 4;
a lysine to glutamic acid substitution at position 100;
a methionine to valine substitution at position 223; and
an asparagine to aspartic acid substitution at position 336.

52. The system according to any one of the claims 48 to 51, wherein the at least one allele is selected from the group consisting of:
the G allele of marker rs1058396;
the C allele of marker rs11877062;
the G allele of marker rs2298720; and
the A allele of marker rs2298719.

53. The system according to any one of claims 48-52, wherein the measurement tool comprises a tool stored on a computer-readable medium of the system and adapted to be executed by a processor of the system to receive a data input about a subject and determine information about the presence or absence of the at least one allele in a human subject from the data.

54. The system according to claim 53, wherein the data is genomic sequence information, and the measurement tool comprises a sequence analysis tool stored on a computer readable medium of the system and adapted to be executed by a processor of the system to determine the presence or absence of the at least one allele from the genomic sequence information.

55. The system according to any one of claims 48-54, wherein the input about the human subject is a biological sample from the human subject, and wherein the measurement tool comprises a tool to identify the presence or absence of the at least one allele in the biological sample, thereby generating information about the presence or absence of the at least one allele in a human subject.

56. The system according to claim 55, wherein the measurement tool includes:
an oligonucleotide microarray containing a plurality of oligonucleotide probes attached to a solid support;
a detector for measuring interaction between nucleic acid obtained from or amplified from the biological sample and one or more oligonucleotides on the oligonucleotide microarray to generate detection data; and
an analysis tool stored on a computer-readable medium of the system and adapted to be executed on a processor of the system, to determine the presence or absence of the at least one allele based on the detection data.

57. The system according to claim 56, wherein the measurement tool includes:
a nucleotide sequencer capable of determining nucleotide sequence information from nucleic acid obtained from or amplified from the biological sample; and
an analysis tool stored on a computer-readable medium of the system and adapted to be executed on a processor of the system, to determine the presence or absence of the at least one allele based on the nucleotide sequence information.

58. The system according to any one of claims 48 to 57, further comprising:
a medical protocol database operatively connected to a computer-readable medium of the system and containing information correlating the presence or absence of the at least one allele and medical protocols for human subjects at risk for bladder cancer; and
a medical protocol routine, operatively connected to the medical protocol database and the analysis routine, stored on a computer-readable medium of the system, and adapted to be executed on a processor of the system, to compare the conclusion from the analysis routine with respect to susceptibility to bladder cancer for the subject and the medical protocol database, and generate a protocol report with respect to the probability that one or more medical protocols in the database will:
reduce susceptibility to bladder cancer; or
delay onset of bladder cancer; or
increase the likelihood of detecting bladder cancer at an early stage to facilitate early treatment.

59. The system according to any one of claims 49-58, wherein the communication tool is operatively connected to the analysis routine and comprises a routine stored on a computer-readable medium of the system and adapted to be executed on a processor of the system, to:
generate a communication containing the conclusion; and transmit the communication to the subject or the medical practitioner, or enable the subject or medical practitioner to access the communication.

60. The system according to claim 59, wherein the communication expresses the susceptibility to bladder cancer in terms of odds ratio or relative risk or lifetime risk.

61. The system according to claim 59 or 60, wherein the communication further includes the protocol report.

62. The system according to any one of claims 48-61, wherein the susceptibility database further includes information about at least one parameter selected from the group consisting of age, sex, ethnicity, race, medical history, weight, diabetes status, blood pressure, family history of bladder cancer, and smoking history in humans and impact of the at least one parameter on susceptibility to bladder cancer.

63. A system for assessing or selecting a treatment protocol for a subject diagnosed with bladder cancer, comprising:
at least one processor;
at least one computer-readable medium;
a medical treatment database operatively connected to a computer-readable medium of the system and containing information correlating the presence or absence of at least one mutant SLC14A1 allele and efficacy of treatment regimens for bladder cancer;
a measurement tool to receive an input about the human subject and generate information from the input about the presence or absence of the at least one SLC14A1 allele in a human subject diagnosed with bladder cancer; and
a medical protocol tool operatively coupled to the medical treatment database and the measurement tool, stored on a computer-readable medium of the system, and adapted to be executed on a processor of the system, to compare the information with respect to presence or absence of the at least one SLC14A1 allele for the subject and the medical treatment database, and generate a conclusion with respect to at least one of:
the probability that one or more medical treatments will be efficacious for treatment of bladder cancer for the patient; and
which of two or more medical treatments for bladder cancer will be more efficacious for the patient.

64. The system according to claim 63, wherein the measurement tool comprises a tool stored on a computer-readable medium of the system and adapted to be executed by a processor of the system to receive a data input about a subject and determine information about the presence or absence of the at least one allele in a human subject from the data.

65. The system according to claim 63, wherein the data is genomic sequence information, and the measurement tool comprises a sequence analysis tool stored on a computer readable medium of the system and adapted to be executed by a processor of the system to determine the presence or absence of the at least one allele from the genomic sequence information.

66. The system according to claim 63, wherein the input about the human subject is a biological sample from the human subject, and wherein the measurement tool comprises a tool to identify the presence or absence of the at least one allele in the biological sample, thereby generating information about the presence or absence of the at least one allele in a human subject.

67. The system according to any one of claims 63-66, further comprising a communication tool operatively connected to the medical protocol routine for communicating the conclusion to the subject, or to a medical practitioner for the subject.

68. The system according to claim 67, wherein the communication tool comprises a routine stored on a computer-readable medium of the system and adapted to be executed on a processor of the system, to:
generate a communication containing the conclusion; and
transmit the communication to the subject or the medical practitioner, or enable the subject or medical practitioner to access the communication.

69. The system according to any one of the claims 63 to 68, wherein the at least one allele is indicative of an amino acid substitution in a protein with sequence as set forth in SEQ ID NO:133, selected from the group consisting of:
an arginine to tryptophan substitution at position 4;
a lysine to glutamic acid substitution at position 100;
a methionine to valine substitution at position 223; and
an asparagine to aspartic acid substitution at position 336.

70. The system according to any one of the claims 63 to 69, wherein the at least one allele is selected from the group consisting of:
the G allele of marker rs1058396;
the C allele of marker rs11877062;
the G allele of marker rs2298720; and
the A allele of marker rs2298719.
